SEQUENCE LISTING (30 sequences)

SEQ ID NO: 1 - DNA, 37 bp, Artificial Sequence
  Splint oligonucleotide seq
acagtttcggagtacgaacatcggtgcgtttcgcaag

SEQ ID NO: 2 - DNA, 82 bp, Artificial Sequence
  Probe oligonucleotide seq
  5Phos (1)..(1): 5′ phosphorylation
  Phosphorothioate-bond (1)..(4): Phosphorothioate bonds between bases 1&2, 2&3, 3&4
atgttcgatgagctttgacaatacttgacgattcatgccgtacatgcagctatgcagatataggatgttgcgaaacgcaccg

SEQ ID NO: 3 - DNA, 20 bp, Artificial Sequence
  Primer mix 1 Fwd
ggcatgaatcgtcaagtatt

SEQ ID NO: 4 - DNA, 20 bp, Artificial Sequence
  Primer mix 1 Rev
atgcagatataggatgttgc

SEQ ID NO: 5 - DNA, 20 bp, Artificial Sequence
  Labeled probe seq
  5Cy5 (1)..(1): Cy5 on 5′ end
  TAO (9)..(10): TAO fluorescent quencher between bases 9&10
  3IAbRQSp (20)..(20): Iowa Black RQ fluorescence quencher on 3′ end
cgcaccgatgttcgatgagc

SEQ ID NO: 6 - DNA, 51 bp, Artificial Sequence
  Capture oligonucleotide seq
  5biotin (1)..(1): Biotin on 5′ end
ttttttttttttttttttttatctttttgaattcagtttccttcatctagg

SEQ ID NO: 7 - DNA, 97 bp, Artificial Sequence
  Probe seq
  5Phos (1)..(1): Phosphorylated 5′ end
  Phosphorothioate-bond (1)..(4): Phosphorothioate bonds between bases 1&2, 2&3, 3&4
atgttcgatgagctttgacaatacttgacgattcatgccgtacatgcagctatgcagatataggatgttgcgaaacgcaccggaggccagcactttg

SEQ ID NO: 8 - DNA, 70 bp, Artificial Sequence
  Wildtype (WT) oligonucleotide seq
aaggaaactgaattcaaaaagatcaaagtgctgggctccggtgcgttcggcacggtgtataaggtaaggt

SEQ ID NO: 9 - DNA, 70 bp, Artificial Sequence
  Mutant oligonucleotide seq
aaggaaactgaattcaaaaagatcaaagtgctggcctccggtgcgttcggcacggtgtataaggtaaggt

SEQ ID NO: 10 - DNA, 77 bp, Artificial Sequence
  Probe seq
  5Phos (1)..(1): Phosphorylated 5′ end
  Phosphorothioate-bond (1)..(1): Phosphorothioate bonds between bases 1&2, 2&3, 3&4
atgttcgatgagctttgacaatacttgacatgcgcagatataggatgttgcgaaacgcaccggaggccagcactttg

SEQ ID NO: 11 - DNA, 20 bp, Artificial Sequence
  Primer mix 1 Fwd
  Phosphorothioate-bonds (1)..(20): Phosphorothioate bonds between bases 1&2, 2&3, 17&18, 18&19, 19&20
tcgcaacatcctatatctgc

SEQ ID NO: 12 - DNA, 20 bp, Artificial Sequence
  Primer mix 1 Rev
  Phosphorothioate-bonds (1)..(10): Phosphorothioate bonds between bases 1&2, 2&3, 17&18, 18&19, 19&20
tgagctttgacaatacttga

SEQ ID NO: 13 - DNA, 21 bp, Artificial Sequence
  Primer mix 2 Fwd
  Phosphorothioate-bonds (18)..(21): Phosphorothioate bonds between bases 18&19, 19&20, 20&21
acatcggtgcgtttcgcaaca

SEQ ID NO: 14 - DNA, 20 bp, Artificial Sequence
  Primer mix 2 Rev
  Phosphorothioate-bonds (1)..(20): Phosphorothioate bonds between bases 1&2, 2&3, 17&18, 18&19, 19&20
tgagctttgacaatacttga

SEQ ID NO: 15 - DNA, 20 bp, Artificial Sequence
  Primer mix Fwd
tcgcaacatcctatatctgc

SEQ ID NO: 16 - DNA, 20 bp, Artificial Sequence
  Primer mix Rev
tgagctttgacaatacttga

SEQ ID NO: 17 - DNA, 70 bp, Artificial Sequence
  WT oligo reverse complementary seq
accttaccttatacaccgtgccgaacgcaccggagcccagcactttgatctttttgaattcagtttcctt

SEQ ID NO: 18 - DNA, 70 bp, Artificial Sequence
  Mutant oligo reverse complementary seq
accttaccttatacaccgtgccgaacgcaccggaggccagcactttgatctttttgaattcagtttcctt

SEQ ID NO: 19 - DNA, 81 bp, Artificial Sequence
  WT oligonucleotide seq
ctgctgggcatctgcctcacctccaccgtgcagctcatcacgcagctcatgcccttcggctgcctcctggactatgtccgg

SEQ ID NO: 20 - DNA, 81 bp, Artificial Sequence
  Mutant oligonucleotide seq
ctgctgggcatctgcctcacctccaccgtgcagctcatcatgcagctcatgcccttcggctgcctcctggactatgtccgg

SEQ ID NO: 21 - DNA, 74 bp, Artificial Sequence
  Probe A0 seq
  5Phos (1)..(1): Phosphorylated 5′ end
  Phosphorothioate-bonds (1)..(4): Phosphorothioate bonds between bases 1&2, 2&3, 3&4
agctgcatctgagctttgacaatacttgagcacggcagatataggatgttgcgaagggcatgagctgcatgatg

SEQ ID NO: 22 - DNA, 20 bp, Artificial Sequence
  Primer mix Fwd
  Phosphorothioate-bonds (1)..(3): Phosphorothioate bonds between bases 1&2, 2&3
tcgcaacatcctatatctgc

SEQ ID NO: 23 - DNA, 20 bp, Artificial Sequence
  Primer mix Rev
atgttgcgaagggcatatgt

SEQ ID NO: 24 - DNA, 49 bp, Artificial Sequence
  Capture oligonucleotide seq
  5biotin (1)..(1): biotin on 5′ end
ttttttttttttttttttttaggcgggagacatatggggagcccagtgg

SEQ ID NO: 25 - DNA, 93 bp, Artificial Sequence
  Probe seq
  5Phos (1)..(1): Phosphorylated 5′ end
  Phosphorothioate-bond (1)..(2): Phosphorothioate bond between residues 1&2
  3SpC3 (93)..(93): C3 spacer on 3′ end
atgttcgatgagctttgacaatacttgatcgatgcagatataggatgttgcgaccatcacgtatgccatcacgtatgcttcctggggacattt

SEQ ID NO: 26 - DNA, 90 bp, Artificial Sequence
  Unmethylated oligonucleotide seq
ctcagcgtacccttgtccccaggaagcatacgtgatggcatacgtgatggctggtgtgggctccccatatgtctcccgccttctgggcat

SEQ ID NO: 27 - DNA, 90 bp, Artificial Sequence; identical to SEQ ID NO: 26
  Methylated oligonucleotide seq
  iMe-dC (20)..(20): 5-Methyl deoxycytidine

SEQ ID NO: 28 - DNA, 36 bp, Artificial Sequence
  Splint oligonucleotide seq
tgtcaaagctcagctatctgacgtgattcgcaacaa

SEQ ID NO: 29 - DNA, 20 bp, Artificial Sequence
  Primer mix 1 Fwd
  Phosphorothioate-bonds (1)..(3): Phosphorothioate bonds between bases 1&2, 2&3
tcgcaacatcctatatctgc

SEQ ID NO: 30 - DNA, 20 bp, Artificial Sequence
  Primer mix 1 Rev
  Phosphorothioate-bonds (1)..(3): Phosphorothioate bonds between bases 1&2, 2&3
tgagctttgacaatacttga

(19) United States
(12) Patent Application Publication
(10) Pub. No.: US 2024/0392345 A1
(43) Pub. Date: Nov. 28, 2024

(54) IMPROVED POLYNUCLEOTIDE SEQUENCE DETECTION METHOD

(71) Applicant: Biofidelity LTD, Cambridge (GB)

(72) Inventors: Barnaby William Balmforth, Cambridge (GB); Cameron Alexander FRAYLING, Cambridge (GB); Ana Luisa BRAS DOS SANTOS RIBEIRO DA SILVA WEATHERLEY, Cambridge (GB); Paulina Klaudyna POWALOWSKA, Cambridge (GB); Magdalena STOLAREK-JANUSZKIEWICZ, Cambridge (GB)

(21) Appl. No.: 18/259,192

(22) PCT Filed: Dec. 23, 2021
(86) PCT No.: PCT/GB2021/053418

§ 371 Date: Jun. 23, 2023

(30) Foreign Application Priority Data
Jan. 28, 2021 (GB) 2020539.9
Jan. 28, 2021 (GB) 2101176.2

(51) Int. Cl. C12Q 1/6816; C12Q 1/6844

(52) U.S. Cl. CPC C12Q 1/6816; C12Q 1/6844

(57) ABSTRACT

Disclosed is an improved polynucleotide sequence detection method suitable for testing for the presence of a large number of diagnostic markers, including those used in the identification of cancer, infectious disease and transplant organ rejection.

### CROSS-REFERENCE TO RELATED APPLICATIONS

This application is a § 371 national phase of International Application No. PCT/GB2021/053418, filed on Dec. 23, 2021, which claims the benefit of United Kingdom Application No. 2020539.9, filed on Dec. 23, 2020, and United Kingdom Application No. 2101176.2, filed on Jan. 28, 2021, which applications are incorporated by reference herein.

### INCORPORATION BY REFERENCE OF SEQUENCE LISTING PROVIDED AS A TEXT FILE

A Sequence Listing is provided herewith in a text file, “BIFI-005_SEQLIST_ST25_Corrected”, created on Jun. 11, 2024, and having a size of 9,435 bytes. The contents of the text file are incorporated herein by reference in its entirety.

This invention relates to an improved polynucleotide sequence detection method suitable for testing for the presence of a large number of diagnostic markers, including those used in the identification of cancer, infectious disease and transplant organ rejection. It is also useful for companion diagnostic testing in which a panel of markers must be identified reliably and at low cost.

The polymerase chain reaction (PCR) is a well-known and powerful technique for amplifying DNA or RNA present in laboratory and diagnostic samples to a point where they can be reliably detected and/or quantified. However, when applied for the purposes of investigating analyte samples containing low-levels of such molecules, it suffers from a number of limitations. First, whilst the technique can detect as little as a single target molecule, it is prone to generating false positive results due to unwanted amplification of other nucleic acid sequences present in the sample. This makes the choice of oligonucleotide primers used to initiate the reaction key; which in turn makes designing primers with the required level of specificity relatively complex. As a consequence, many PCR-based tests available on the market today have limited specificity.

A second drawback is that multiplexing of PCR-based methods is in practice limited to at most tens of target sequences (frequently no more than 10) with the avoidance of primer-primer interactions resulting in the need for relatively narrow operational windows.

Another issue is that, because the PCR reaction cycles in an exponential fashion, quantification of the target is difficult; small variations in the efficiency of the reaction having a huge impact on the amount of detectable material generated. Even with appropriate controls and calibrations in place, quantification is thus typically limited to an accuracy within a factor of around 3.

Finally, mutations in the region targeted for investigation by PCR amplification methods can have unwanted side effects. For example, there have been instances where FDA-approved tests have had to be withdrawn because the target organism underwent mutation in the genetic region targeted by the test primers resulting in large numbers of false negatives. Conversely, if a specific single nucleotide polymorphism (SNP) is targeted for amplification the PCR method will often give a false positive when the wild-type variant is present. Avoiding this requires very careful primer design and further limits the efficacy of multiplexing. This is particularly relevant when searching for panels of SNPs as is a common requirement in cancer testing/screening or companion diagnostics.

US2006/110765 A1 (Wang et al) teaches enzymatic cleavage at a mismatch, which is typically an inefficient and not a highly specific reaction. Moreover, off target hybridisation of the probes as disclosed in Wang et al to similar sequences in a sample would result in false positive results because of the use of cleavage at mismatches. It would also not be possible to distinguish between two different genetic variants in the same or near-neighbouring positions as they would all result in cleavage and amplification of the same probe. The teachings of Wang et al would therefore result in low sensitivity and specificity of the reaction scheme. In contrast, the technical effect of the method as disclosed by the present invention provides a fast, efficient method with high specificity to dsDNA, which can be effectively blocked by a mismatch. In addition, the method of the present invention is extremely specific to the targeted genetic variant, enabling discrimination between different variants in the same or near-neighbouring positions.

US2009/239283 A1 (Liu et al) teaches the use of non-extendable 3′ ends which are removed by pyrophosphorolysis, necessitating the genetic engineering of custom polymerases capable of removing the 3′ blocking modification. In contrast the present invention utilises the natural pyrophosphorolysis activity inherent in existing polymerases and does not use 3′ blocking modifications. The method as disclosed in Liu et al also relies on the removal of only the terminal base from a fraction of the probe to enable subsequent amplification, and is limited to this embodiment by the use of 3′ blocking modification. In contrast, the methods disclosed in the present invention enables embodiments, in which progressive removal of multiple bases from the probe is required to set off the reaction, making it substantially more robust to transient off-target annealing either to the background DNA or to other probes, which can result in the unwanted removal of the terminal base.

WO2020/016590 describes a method for detecting a target nucleic acid sequence in which a sample is contacted with a single stranded probe, the probe digested with a pyrophosphorolysing enzyme if complementary to the target, and the digested probe detected. The method takes place in solution and uses multiple steps of pyrophosphorolysis and ligation of detect the target sequence. The invention below is a simplified version of the assays disclosed therein using fewer enzymes.

Ingram et al (“PAP-LMPCR for improved, allele-specific footprinting and automated chromatin fine structure analysis”, NUCLEIC ACIDS RESEARCH, vol. 36, n. 3, 21 Jan. 2008) teaches a method wherein a ligation reaction is very inefficient in the presence of a pyrophosphorolysing inducing buffer. The current inventors have surprisingly found an improvement to the method of WO2020/016590 comprising a combined pyrophosphorolysis and ligation step which proceeds efficiently, which the disclosure of Ingram et al teaches away from.

### SUMMARY OF INVENTION

We have now developed an improved method which builds on our experience using the pyrophosphorolysis method employed in our earlier patent (WO/2020/016590) to overcome many of these limitations. In doing so, it harnesses the double-strand specificity of pyrophosphorolysis; a reaction which will not proceed efficiently with single-stranded oligonucleotide substrates or double-stranded substrates which include blocking groups or nucleotide mismatches. The new method is faster, less complex and less expensive to run than that disclosed in WO/2020/016590. Furthermore, the methods of the current invention offer superior sensitivity over those of WO/2020/016590 by reducing the background signal. Thus, according to the present invention, there is provided a method for the detection of a polynucleotide target sequence, comprising the steps of:
a. introducing a sample comprising one or more nucleic acid analytes to a first reaction mixture comprising:
   i. a single-stranded probe oligonucleotide A₀ including a region complementary to a target nucleic acid sequence;
   ii. a capture oligonucleotide B₀ comprising a region complementary to a region adjacent to the target nucleic acid sequence and a capture moiety;
   iii. a solid support,
b. allowing A₀ and B₀ to hybridise to the nucleic acid analyte;
c. introducing the hybridised probes A₀ to a second reaction mixture comprising a pyrophosphorolysing enzyme, wherein A₀ is pyrophosphorolysed in the 3′-5′ direction from the 3′ end to create at least a partially digested strand A₁;
d. introducing the partially digested strand A₁ to a third reaction mixture comprising a ligase, wherein A₁ undergoes ligation to form A₂; and
e. detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

Steps c and d can be combined. Thus included herein is a method for the detection of a polynucleotide target sequence in a sample, comprising the steps of:
a. Introducing a sample comprising one or more nucleic acid analytes to a first reaction mixture comprising:
   i. a single-stranded probe oligonucleotide A₀ including a region complementary to a target nucleic acid sequence;
   ii. a capture oligonucleotide B₀ comprising a region complementary to a region adjacent to the target nucleic acid sequence and a capture moiety;
   iii. a solid support,
b. allowing A₀ and B₀ to hybridise to the nucleic acid analyte;
c. introducing the hybridised probes A₀ to a second reaction mixture comprising a pyrophosphorolysing enzyme and a ligase, wherein A₀ is pyrophosphorolysed in the 3′-5′ direction from the 3′ end to create at least a partially digested strand A₁ and A₁ undergoes ligation to form A₂; and
detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

The combination of steps c and d is faster, less complex and less expensive to run than assays disclosed in WO/2020/016590.

The analytes to which the method of the invention can be applied are those nucleic acids, such as naturally-occurring or synthetic DNA or RNA molecules, which include the target polynucleotide sequence(s) being sought. In some embodiments, the analyte will typically be present in an aqueous solution containing it and other biological material and in some embodiments the analyte will be present along with other background nucleic acid molecules which are not of interest for the purposes of the test. In some embodiments, the analyte will be present in low amounts relative to these other nucleic acid components. Suitably, the analyte is derived from a biological sample taken from a mammalian subject (especially a human patient) such as blood, plasma, sputum, urine, skin or a biopsy. In some embodiments, the biological sample will be subjected to lysis in order that the analyte is released by disrupting any cells present. In other embodiments, the analyte may already be present in free form within the sample itself; for example cell-free DNA circulating in blood or plasma.

### BRIEF DESCRIPTION OF DRAWINGS

FIG. 1: Protocols for simplified polynucleotide sequence detection methods.

FIG. 2: The Influence of pyrophosphorolysis (PPL) enzyme choice on reaction sensitivity when using ligated probe. With addition of Bsu L.F. and Bst 2.0 WS the sensitivity of 10 aM of ligated probe is achieved, comparable to sensitivity when no enzyme is present. Bst L.F., Klenow and Mako decrease the sensitivity of the reaction.

FIG. 3: Sensitivity of the Target Enrichment protocol is 100 fM of mutant target at 0.1% AF.

FIG. 4(A)-4(B): Detection of G719X_6239 mutation. Fluorescent measurement results showing mutant signal above wild type obtained from oligonucleotides attached to beads from which the excess of unbound probe was washed away. As described in Example 4.

FIG. 5(A)-5(B): Detection of G719X_6239 mutation. Fluorescent measurement results showing mutant signal above wild type obtained from oligonucleotides in solution. As described in Example 4.

FIG. 6(A)-6(B): Fluorescent signal observed above that of wild-type background DNA in the presence of the G719A mutation in the human EGFR gene at a concentration of 0.001 pM when using a bead-based enrichment method (A) compared to that observed without enrichment in the presence of 25 pM of the same mutation (B). An exemplary workflow for one such bead based target enrichment embodiment is described in Example 5.

FIG. 7: A schematic representation of the circularisation of A₁ to form A₂ against the analyte target sequence. A₀ is progressively digested against the target in the 3′-5′ direction from the 3′ end of A₀ to form partially digested strand A₁, this is shown as steps (A) and (B). This progressive digestion reveals the region of the target that is complementary to the 5′ end of A₀/A₁ and the 5′ end of A₁ then hybridises to this region, this is shown in step (C). A₁ is then ligated together to form circularised A₂, step (D).

FIG. 8: Fluorescence measurement results for Example 6 showing results from an embodiment wherein pyrophosphorolysis of A₀ to form A₁ occurs followed by circularisation of A₁ to form A₂ against a target sequence.

FIG. 9: A single-stranded probe oligonucleotide A₀ anneals to a target polynucleotide sequence to create a first intermediate product which is at least partially double-stranded and in which the 3′ end of A₀ forms a double-stranded complex with the target polynucleotide sequence. In this simplified embodiment of the invention there are two molecules of A₀ present and one target polynucleotide sequence, in order to illustrate how A₀ that has not annealed to a target does not take part in further steps of the method. In this illustrative example, the 3′ end of A₀ anneals to the target polynucleotide sequence whilst the 5′ end of A₀ does not. The 5′ end of A₀ comprises a 5′ chemical blocking group, a common priming sequence and a barcode region.

The partially double-stranded first intermediate product undergoes pyrophosphorolysis in the presence of a pyrophosphorolysing enzyme in the 3′-5′ direction from the 3′ end of A₀ to create a partially digested strand A₁, the analyte and the undigested A₀ molecule which did not anneal to a target.

FIG. 10: A₁ is annealed to a single-stranded trigger oligonucleotide B and the A₁ strand is extended in the 5′-3′ direction against B to create an oligonucleotide A₂. In this illustrative example, trigger oligonucleotide B has a 5′ chemical block. Any undigested A₀ anneals to the trigger oligonucleotide B, however it is unable to be extended in the 5′-3′ direction against B to generate sequences that are the targets for later parts of the method. In this example, A₂ is primed with at least one single-stranded primer oligonucleotide and multiple copies of A₂, or a region of A₂ are created.

FIG. 11: A₁ is annealed to a splint oligonucleotide D, and then circularised by ligation of its 3′ and 5′ ends. The now circularised A₂ is primed with at least one single-stranded primer oligonucleotide and multiple copies of A₂, or a region of A₂ are created. In this illustrative example, the splint oligonucleotide D is unable to extend against A₁ by virtue of either a 3′-modification (chemical in this illustration) or through a nucleotide mismatch between the 3′ end of D and the corresponding region of A₂.

FIG. 12: The 3′ region of a splint oligonucleotide D anneals to the 3′ region of A₁ whilst the 5′ region of the splint oligonucleotide D anneals to the 5′ region of a ligation probe C. Thus, a second intermediate product A₂ is formed comprised of A₁, C and optionally an intermediate region formed by extension of A₁ in the 5′-3′ direction to meet the 5′ end of C. In this illustrative example, the ligation probe C has a 3′ chemical blocking group so that a 3′-5′ exonuclease can be used to digest any non-ligated A₁.

A₂ is primed with at least one single-stranded primer oligonucleotide and multiple copies of A₂, or a region of A₂ are created.

FIG. 13: Comparison of detection of 5% methylated strands in the presence of unmethylated strands using targeted enrichment with beads (FIG. 13A) and oligonucleotides in solution (FIG. 13B). The detection of 100 fM methylated target when using bead based target enrichment, compared to 10 pM when oligonucleotides are in solution.

### DESCRIPTION OF EMBODIMENTS

In an aspect of the present invention, there is provided a method for the detection of a polynucleotide target sequence in a sample, comprising the steps of:
a. introducing one or more nucleic acid analytes to a first reaction mixture comprising:
   i. a single-stranded probe oligonucleotide A₀ including a region complementary to a target nucleic acid sequence;
   ii. a capture oligonucleotide B₀ comprising a region complementary to a region adjacent to the target nucleic acid sequence and a capture moiety;
   iii. a solid support,
b. allowing A₀ and B₀ to hybridise to the nucleic acid analyte;
c. introducing the hybridised probes A₀ to a second reaction mixture comprising a pyrophosphorolysing enzyme, wherein A₀ is pyrophosphorolysed in the 3′-5′ direction from the 3′ end to create at least a partially digested strand A₁;
d. introducing the partially digested strand A₁ to a third reaction mixture comprising a ligase, wherein A₁ undergoes ligation to form A₂; and
e. detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

In one embodiment, the 3′ end of A₀ is perfectly complementary to the target polynucleotide sequence.

In one embodiment, the ligase is substantially lacking in single-strand ligation activity.

In some embodiments, the reaction mixture comprising partially digested strand A₁ is introduced to an inorganic pyrophosphatase prior to introduction to the third reaction mixture.

In some embodiments, the reaction mixture comprising partially digested strand A₁ is introduced to an inorganic pyrophosphatase at the same time as introduction to the third reaction mixture.

In some embodiments, the reaction mixture comprising partially digested strand A₁ is introduced to an inorganic pyrophosphatase following introduction to the third reaction mixture.

In some embodiments, the third reaction mixture further comprises an inorganic pyrophosphatase.

In some embodiments, the second and third reaction mixtures are combined and thus the method comprises the steps of:
a. introducing a sample comprising one or more nucleic acid analytes to a first reaction mixture comprising:
   i. A single-stranded probe oligonucleotide A₀ including a region complementary to a target nucleic acid sequence;
   ii. A capture oligonucleotide B₀ comprising a region complementary to a region adjacent to the target nucleic acid sequence and a capture moiety;
   iii. A solid support.
b. allowing A₀ and B₀ to hybridise to the nucleic acid analyte;
c. introducing the hybridised probes A₀ to a second reaction mixture comprising a pyrophosphorolysing enzyme and a ligase, wherein A₀ is pyrophosphorolysed in the 3′-5′ direction from the 3′ end to create at least a partially digested strand A₁ and wherein A₁ undergoes ligation to form A₂; and
d. detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

In some embodiments, the second reaction mixture further comprises a source of pyrophosphate ion.

In some embodiments, B₀ is attached to the solid support, prior to step (b).

In some embodiments, B₀ is attached to the solid support between steps (b) and (c).

In some embodiments, following attachment of B₀ to the solid support and hybridisation of A₀ and B₀ to the target, the supernatant and thus any A₀ which are not hybridised to the target nucleic acid analyte are removed from the reaction mixture.

This removal can occur prior to (c).

This removal can occur prior to (d).

In some embodiments, the capture oligonucleotide is complementary to a region that is within 10000, 5000, 2500, 1000, 500, 250, 100, 90, 80, 70, 60, 50, 40, 30, 20, 10 or 5 nucleotides of the target nucleic acid sequence.

In some embodiments, the capture oligonucleotide is complementary to a region that is within 100 nucleotides of the target nucleic acid sequence.

In some embodiments, the capture oligonucleotide is complementary to a region that is within 50 nucleotides of the target nucleic acid sequence.

In some embodiments, the capture oligonucleotide is complementary to a region that is within 10 nucleotides of the target nucleic acid sequence.

In some embodiments, the oligonucleotides A₀ and B₀ are joined to form a single oligonucleotide.

In some embodiments, the solid support is a polymer and/or resin coated solid surface.

In some embodiments, the solid support is a polystyrene solid support.

In some embodiments, polystyrene (C₈H₈)ₙ is a polymer wherein n is 10, 20, 30, 40, 50, 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 12,500, 15,000, 16,000, 17,000, 17,500, 18,000, 19,000, 20,000, 25,000, 30,000, 40,000, 50,000, 60,000, 70,000, 80,000, 90,000, 100,000 or more, or where n is any integer between any of these points, or n is within any range derivable between any two of these points, is utilised.

In some embodiments, the polystyrene solid support is a particle, micro particle, magnetic bead, resin or any particulate that comprises polystyrene polymers.

In some embodiments, the polystyrene support is modified to include one or more of the following functional groups: amine, carboxylate, sulfonate, trimethylamine and/or epoxide.

In some embodiments, the solid support is a magnetic bead.

In some embodiments, the magnetic bead is a shape which maximises the surface area of said bead.

In some embodiments, the magnetic bead is a regular shape.

In some embodiments, the magnetic bead is an irregular shape.

In some embodiments, the magnetic bead has a diameter of less than, or equal to: 1000, 900, 800, 700, 600, 500, 400, 300, 200, 100, 90, 80, 70, 60, 50, 40, 30, 20, 10, 5, 2.5, 1, 0.5, 0.25 or 0.1 micron.

In some embodiments, the solid support is a magnetic polystyrene bead.

In some embodiments, the magnetic polystyrene bead comprises iron oxide.

In some embodiments, the magnetic polystyrene bead is Streptavidin-coupled.

In some embodiments, the solid support is a Streptavidin-coupled Dynabead (RTM).

In some embodiments, the solid support is a dextran-modified surface.

In some embodiments, the dextran-modified surface is a particle, micro particle, magnetic bead, resin or any particulate that comprises dextran polymers.

In some embodiments, the dextran polymers have an approximate molecular weight from 1000 to 410000.

In some embodiments, the dextran polymers have an approximate molecular weight from 25000 to about 100000.

In some embodiments, the dextran-surface modified is further modified to include one or more functional groups.

In some embodiments, the dextran-modified surface is modified to include one or more of the following functional groups: amine, carboxylate, sulfonate, trimethylamine and/or epoxide.

In some embodiments, the solid support is a magnetic bead.

In some embodiments, the magnetic bead is a shape which maximises the surface area of said bead.

In some embodiments, the magnetic bead is a regular shape.

In some embodiments, the magnetic bead is an irregular shape.

In some embodiments, the magnetic bead has a diameter of less than, or equal to: 1000, 900, 800, 700, 600, 500, 400, 300, 200, 100, 90, 80, 70, 60, 50, 40, 30, 20, 10, 5, 2.5, 1, 0.5, 0.25 or 0.1 micron.

In some embodiments, the magnetic bead is a dextran magnetic bead selected from: Nanomag® Dextran (ND); Nanomag® Dextran-SO3H (ND-SO3H); BioMag® Dextran-Coated Charcoal; or BioMag® Plus Dextran.

In some embodiments, the solid support is a Polyethylene Glycol (PEG) or PEG-modified surface.

In some embodiments, the Polyethylene Glycol (PEG) or PEG-modified surface is a particle, micro particle, magnetic bead, resin or any particulate that comprises PEG.

In some embodiments of the invention PEG, (CH₂O)ₙ, is a polymer wherein n is 10, 20, 30, 40, 50, 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 12,500, 15,000, 16,000, 17,000, 17,500, 18,000, 19,000, 20,000, 25,000, 30,000, 40,000, 50,000, 60,000, 70,000, 80,000, 90,000, 100,000 or more, or where n is any integer between any of these points, or n is within any range derivable between any two of these points, is utilised.

In some embodiments, the PEG utilised is PEG-200, PEG-300, PEG-400, PEG-600, PEG-1000, PEG-1300-1600, PEG-1450, PEG-3000-3700, PEG-3500, PEG-6000, PEG-8000 or PEG-17500.

In some embodiments, wherein the Polyethylene Glycol (PEG) or PEG-modified surface is a magnetic microparticle or bead, the microparticle or bead is selected from Nanomag® PEG-300 (Plain) or Nanomag®-D.

In some embodiments, the solid support is a Polyvinylpyrrolidone (PVP) or PVP-modified surface.

In some embodiments, the PVP or PVP-modified surface is a particle, micro particle, magnetic bead, resin or any particulate that comprises PVP.

In some embodiments of the invention PVP, n-vinyl pyrrolidone, is a polymer wherein n is 10, 20, 30, 40, 50, 100, 150, 200, 250, 300, 350, 400, 450, 500, 550, 600, 650, 700, 750, 800, 850, 900, 950, 1,000, 1,250, 1,500, 1,750, 2,000, 2,500, 3,000, 3,500, 4,000, 4,500, 5,000, 5,500, 6,000, 6,500, 7,000, 7,500, 8,000, 8,500, 9,000, 9,500, 10,000, 11,000, 12,000, 12,500, 15,000, 16,000, 17,000, 17,500, 18,000, 19,000, 20,000, 25,000, 30,000, 40,000, 50,000, 60,000, 70,000, 80,000, 90,000, 100,000 or more, or where n is any integer between any of these points, or n is within any range derivable between any two of these points, is utilised.

In some embodiments, the solid support is a polysaccharide or polysaccharide-modified surface.

In some embodiments, the polysaccharide or polysaccharide-modified surface is a particle, micro particle, magnetic bead, resin or any particulate that comprises a polysaccharide.

In some embodiments, the polysaccharide is selected from one or more of dextran, ficoll, glycogen, gum arabic, xanthan gum, carageenan, amylose, agar, amylopectin, xylans and/or beta-glucans.

In some embodiments, the solid support is a chemical resin or chemical resin-modified surface.

In some embodiments, the chemical resin or chemical-resin modified surface is selected from one or more of the following resins: isocyanate, glycerol, piperidino-methyl, polyDMAP (polymer-bound dimethyl 4-aminopyridine), DIPAM (Diisopropylaminomethyl, aminomethyl, polystyrene aldehyde, tris(2-aminomethyl) amine, morpholino-methyl, BOBA (3-Benzyloxybenzaldehyde), triphenyl-phosphine or benzylthio-methyl.

In some embodiments, B₀ is covalently attached to the solid support via the capture moiety of B₀.

In some embodiments, the capture moiety of B₀ is covalently attached to the solid support via a chemically-cleavable linker, such as a disulfide, allyl, or azide-masked hemiaminal ether linker.

In some embodiments, the capture moiety of B₀ is covalently attached to the solid support via amide or phosphorothioate bonds.

The person skilled in the art will appreciate that there exists a plethora of techniques for the covalent and non-covalent immobilisation of oligonucleotides to solid supports, see for example “Strategies for Attaching Oligonucleotides to Solid Supports.” (2014) produced by Integrated DNA Technologies (IDT®).

In some embodiments, B₀ is non-covalently attached to the solid support via the capture moiety of B₀.

The person skilled in the art will appreciate that there exists a plethora of techniques for the covalent and non-covalent immobilisation of oligonucleotides to solid supports, see for example “Strategies for Attaching Oligonucleotides to Solid Supports.” (2014) produced by Integrated DNA Technologies (IDT®).

In some embodiments, the capture moiety of B₀ comprises an oligonucleotide sequence and the solid support comprises oligonucleotides bearing the complementary sequence.

In some embodiments, the length of the complementary sequence is between 10, 20, 30, 40, 50, 100, 150 and 200 bases.

In some embodiments, the length of the complementary sequence is between 10, 20, 30, 40, 50, and 100 bases.

In some embodiments, the length of the complementary sequence is between 10-20, 10-30, 10-40 and 10-50 bases.

In some embodiments, the length of the complementary sequence is between 10-20, 10-30 and 10-40 bases.

In some embodiments, the length of the complementary sequence is between 10-20 and 10-30 bases.

In some embodiments, the length of the complementary sequence is between 10-20 bases.

In some embodiments, the capture moiety comprises a chemical modification to B₀ and B₀ is attached to the solid support via an interaction between the chemical modification and the solid support.

In some embodiments, the chemical modification is biotin and the solid support further comprises streptavidin.

In some embodiments of the method, the first reaction mixture comprises multiple different oligonucleotides A₀ and B₀ and wherein the successfully hybridised A₀ oligonucleotides are simultaneously enriched.

In some embodiments, prior to step (c) A₀, the target nucleic acid, and optionally B₀ are released from the solid support.

In some embodiments, following step (c) A₁, optionally B₀ and optionally the target nucleic acid are released from the solid support.

In some embodiments, during step (c) A₁, optionally B₀ and optionally the target nucleic acid are released from the solid support.

It should be understood that references to release of A₀ encompass embodiments wherein A₀ is converted to A₁ or A₂ whilst solid surface bound and then released.

In some embodiments, A₀, B₀ and the target nucleic acid are released from the solid support by the cleavage of a chemical linker through the addition of tris(2-carboxyethyl)phosphine (TCEP) or dithiothreitol (DTT) for a disulfide linker; palladium complexes or an allyl linker; or TCEP for an azide-masked hemiaminal ether linker.

In some embodiments, A₀, B₀ and the target nucleic acid are released from the solid support by removing a non-canonical base, from A₀ or B₀, and cleavage at the resultant abasic site. In some embodiments, the non-canonical base is uracil, which is removed by uracil DNA glycosylase. In an alternate embodiment, the non-canonical base is 8-oxoguanine, which is removed by formamidopyrimidine DNA glycosylase (Fpg).

In some embodiments, the capture moiety is an oligonucleotide region and release is performed through heating of the reaction mixture.

In some embodiments, the reaction mixture is heated to 37° C.-100° C. over 1-20 minutes.

In some embodiments, the reaction mixture is heated over 1-15 minutes.

In some embodiments, the reaction mixture is heated over 1-10 minutes.

In some embodiments, the reaction mixture is heated over 1-5 minutes.

In some embodiments, the reaction mixture is heated over 5 minutes.

In some embodiments, the reaction mixture is heated to 37° C.-85° C.

In some embodiments, the reaction mixture is heated to 37° C.-75° C.

In some embodiments, the reaction mixture is heated to 37° C.-65° C.

In some embodiments, the reaction mixture is heated to 37° C.-55° C.

In some embodiments, the reaction mixture is heated to 37° C.-45° C.

The person skilled in the art will appreciate that the temperature to which the reaction mixture is heated so enact release of complementary oligonucleotide regions depends on the length of said regions.

In some embodiments, release is achieved through the cleavage of oligonucleotide B₀. This cleavage can be achieved by any of the means previously, or subsequently, described or by any of the means known to the person skilled in the art.

In some embodiments, B₀ is cleaved chemically.

In some embodiments, B₀ is cleaved enzymatically.

In some embodiments, B₀ is cleaved by a restriction enzyme.

In some embodiments, B₀ is cleaved by epigenetic modification sensitive or dependent restriction enzymes.

In some embodiments, B₀ is cleaved by methylation sensitive or dependent restriction enzymes.

In some embodiments, B₀ is cleaved by hydroxymethylation sensitive or dependent restriction enzymes.

In some embodiments, the restriction enzymes are endonucleases.

In some embodiments, B₀ is cleaved by a flap endonuclease.

In some embodiments, B₀ comprises a photocleavable linker and A₀, B₀ and the target nucleic acid are released from the solid support by cleavage of this linker.

In some embodiments, B₀ comprises a UV cleavable linker and A₀, B₀ and the target nucleic acid are released from the solid support by cleavage of this linker.

In some embodiments, release is achieved through the cleavage of A₀ with a methylation-sensitive or methylation-dependent restriction enzyme. In some embodiments, release is achieved through the cleavage of A₀ and the nucleic acid analyte.

In some embodiments, wherein A₀ and B₀ are regions of the same oligonucleotide C₀, C₀ is cleaved with a methylation-sensitive or methylation-dependent restriction enzyme and the portion comprising A₀ and the nucleic acid analyte are released from the solid support.

In some embodiments, release occurs prior to pyrophosphorolysis.

In some embodiments, release occurs simultaneously with pyrophosphorolysis. In some embodiments, target nucleic acid sequence hybridised A₀ is prevented from undergoing pyrophosphorolysis, prior to release from the solid support, by the presence of modifications or mismatches at its 3′ end. In some embodiments, cleavage occurs in the 5′ direction from these modifications or mismatches, thus creating a free 3′ end which allows the pyrophosphorolysis reaction to proceed.

In some embodiments, cleavage and release only occurs in the presence of methylation in the target nucleic acid sequence.

In some embodiments, cleavage and release only occurs in the absence of methylation in the target nucleic acid sequence.

In some embodiments, cleavage and release only occurs in the presence of hydroxymethylation in the target nucleic acid sequence.

In some embodiments, cleavage and release only occurs in the absence of hydroxymethylation in the target nucleic acid sequence.

In some embodiments, cleavage and release only occurs in the presence of an epigenetic modification in the target nucleic acid sequence.

In some embodiments, cleavage and release only occurs in the absence of an epigenetic modification in the target nucleic acid sequence.

In the chemical sciences, methylation denotes the addition of a methyl group to a substrate or the substitution of an atom or group by a methyl group. Methylation is a form of alkylation with specifically a methyl group, rather than a larger carbon chain, replacing a hydrogen atom. These terms are commonly used in chemistry, biochemistry, soil science, and the biological sciences.

In biological systems, methylation is catalysed by enzymes: such methylation can be involved in modification of heavy metals, regulation of gene expression, regulation of protein function, and RNA metabolism. Methylation of heavy metals can also occur outside of biological systems.

Chemical methylation of tissue samples is also one method for reducing certain histological staining artefacts.

Aberrant DNA methylation profiles have been associated many different complex disease states.

In oncology, hypermethylation of tumour suppressor genes in serum DNA can be used as a diagnostic marker for small-cell lung cancer. In patients with immune disease, such as diabetes, rheumatoid arthritis (RA) and systemic lupus erythematosus (SLE), abnormal DNA methylation of cells of the immune system is found. Differential DNA methylation of peripheral blood leukocytes (PBL) in repetitive elements ALU, LINE-1 and Satellite 2 (measures for global DNA methylation), have been found to be associated with ischemic heart disease.

DNA methylation in vertebrates typically occurs at CpG sites (Cytosine-phosphate-guanine sites; that is, where a cytosine is directly followed by a guanine in the DNA sequence); this methylation results in the conversion of the cytosine to 5-methylcytosine. The formation of Me-CpG is catalysed by the enzyme DNA methyltransferase. The bulk of mammalian DNA has about 40% of CpG sites methylated but there are certain areas, known as CpG islands which are GC rich (made up of about 65% CG residues) where none are methylated. These are associated with the promoters of 56% of mammalian genes, including all ubiquitously expressed genes. 1-2% of the human genome is CpG clusters and there is an inverse relationship between CpG methylation and transcriptional activity.

DNA methylation involves the addition of a methyl group to the 5 position of cytosine pyrimidine ring or the 6 nitrogen of the adenine purine ring. This modification can be inherited through cell division. DNA methylation is typically removed during zygote formation and re-established through successive cell divisions during development. DNA methylation is a crucial part of normal organism development and cellular differentiation in higher organisms. DNA methylation stably alters the gene expression pattern in cells such that cells can “remember where they have been”; in other words, cells programmed to be pancreatic islets during embryonic development remain pancreatic islets throughout the life of the organisms without continuing signals telling them that they need to remain islets. In addition, DNA methylation suppresses the expression of viral genes and other deleterious elements which have been incorporated into the genome of the host over time. DNA methylation also forms the basis of chromatin structure, which enables cells to form the myriad characteristics necessary for multicellular life from a single immutable sequence of DNA. DNA methylation also plays a crucial role in the development of nearly all types of cancer.

Bisulfite sequencing is the use of bisulfite treatment of DNA to determine its pattern of methylation. DNA methylation was the first discovered epigenetic mark, and remains the most studied. It is also implicated in repression of transcriptional activity.

Among a number of mRNA modifications, N6-methyladenosine (m6A) modification is the most common type in eukaryotes and nuclear-replicating viruses. m6A has a significant role in numerous cancer types, including leukaemia, brain tumours, liver cancer, breast cancer and lung cancer.

Whilst 5-methylcytosine (5 mC) is the most studied epigenetic modification, 5 mC is oxidised to 5-hydroxymethylcytosine (5 hmC) with the catalysis of TET (ten-eleven translocation) enzymes.

Studies have shown that the distribution of 5 hmC is tissue-specific, and there are differences in the distribution of 5 hmC in different organs and tissues. The decreased expression of 5 hmC in malignant tissue has been shown consistently in a wide range of different cancers, including melanoma. By evaluating a total of 15 pairs of normal and carcinoma samples in human breast tissue, the studies have shown that the levels of 5 hmC were dramatically reduced in the cancer group compared with the healthy breast tissues.

Treatment of DNA with bisulfite converts cytosine residues to uracil, but leaves 5-methylcytosine residues unaffected. Thus, bisulfite treatment introduces specific changes in the DNA sequence that depend on the methylation status of individual cytosine residues, yielding single-nucleotide resolution information about the methylation status of a segment of DNA. Various analyses can be performed on the altered sequence to retrieve this information. The objective analysis is therefore reduced to differentiating between single nucleotide polymorphisms (cytosine's and thymines) resulting from bisulfite conversion. 5 hmC converts to 5 mC upon bisulfite treatment, which then reads as a C when sequenced, and thus cannot distinguish between 5 hmC and 5 mC. The output from bisulfite sequencing can no longer be defined as solely DNA methylation, as it is the composite of 5 mC and 5 hmC. The development of Tet-assisted oxidative bisulfite sequencing is now able to distinguish between the two modifications at single base resolution.

5 hmC can be detected using TET-assisted bisulfite sequencing (TAB-seq). Fragmented DNA is enzymatically modified using sequential T4 Phage R-glucosyltransferase (T4-BGT) and then Ten-eleven translocation (TET) dioxygenase treatments before the addition of sodium bisulfite. T4-BGT glucosylates 5 hmC to form beta-glucosyl-5-hydroxymethylcytosine (5 ghmC) and TET is then used to oxidize 5 mC to 5 caC. Only 5 ghmC is protected from subsequent deamination by sodium bisulfite and this enables 5 hmC to be distinguished from 5 mC by sequencing.

Oxidative bisulfite sequencing (oxBS) provides another method to distinguish between 5 mC and 5 hmC. The oxidation reagent potassium perruthenate converts 5 hmC to 5-formylcytosine (5 fC) and subsequent sodium bisulfite treatment deaminates 5 fC to uracil. 5 mC remains unchanged and can therefore be identified using this method.

APOBEC-coupled epigenetic sequencing (ACE-seq) excludes bisulfite conversion altogether and relies on enzymatic conversion to detect 5 hmC. With this method, T4-BGT glucosylates 5 hmC to 5 ghmC and protects it from deamination by Apolipoprotein B mRNA editing enzyme subunit 3A (APOBEC3A). Cytosine and 5 mC are deaminated by APOBEC3A and sequenced as thymine.

TET-assisted 5-methylcytosine sequencing (TAmC-seq) enriches for 5 mC loci and utilizes two sequential enzymatic reactions followed by an affinity pull-down. Fragmented DNA is treated with T4-BGT which protects 5 hmC by glucosylation. The enzyme mTET1 is then used to oxidize 5 mC to 5 hmC, and T4-BGT labels the newly formed 5 hmC using a modified glucose moiety (6-N3-glucose). Click chemistry is used to introduce a biotin tag which enables enrichment of 5 mC-containing DNA fragments for detection and genome wide profiling.

Methods of methylome analysis are broadly divided into 3 groups: restriction enzyme based, chromatin immunoprecipitation based (ChIP) or affinity based and bisulfite conversion (gene based). Restriction enzyme based methods are methylation-sensitive restriction enzymes for small/large scale DNA methylation analysis by combining the use of methylation-sensitive restriction enzymes experimental approaches (RLGS, DMH etc.) for global methylation analysis, applied to any genome without knowing the DNA sequence. However, large amounts of genomic DNA are required, making the method unsuitable for the analysis of samples when small amount of DNA is recovered. On the other hand, ChIP based methods are useful for the identification of differential methylated regions in tumours through the precipitation of a protein antigen out of a solution by using an antibody directed against the protein. These methods are protein based, applied extensively in cancer research.

Affinity enrichment is a technique that is often used to isolate methylated DNA from the rest of the DNA population. This is usually accomplished by antibody immunoprecipitation methods or with methyl-CpG binding domain (MBD) proteins.

Methylated DNA immunoprecipitation (MeDIP) is an antibody immunoprecipitation method that utilises a 5-methylcytidine antibody to specifically recognise methylated cytosines. The MeDIP kit requires the input DNA sample to be single-stranded in order for the 5-methylcytidine (5-mC) antibody to bind.

Another method for the enrichment of methylated DNA fragments uses recombinant methyl-binding protein MBD2b, or the MBD2b/MBD3L1 complex. One advantage of a methyl-CpG binding protein enrichment strategy is the input DNA sample does not need to be denatured; the protein can recognise methylated DNA in its native double-strand form. Another advantage is that the MBD protein binds only to DNA methylated in a CpG context to ensure the enrichment of methylated-CpG DNA, making this technique ideal for studying CpG islands.

In some embodiments, prior to step (a) of the methods of the invention, the one or more nucleic acid analytes are selectively modified.

In some embodiments, prior to step (a) the unmethylated cytosine bases in the one or more nucleic acid analytes are chemically or enzymatically converted.

In one embodiment, unmodified cytosine bases are converted to uracil by a methyltransferase enzyme.

In one embodiment, this enzyme is M.Sssl.

In one embodiment, unmodified cytosine bases are converted to uracil by a deaminase enzyme.

An enzymatic methyl-seq workflow relies on the ability of APOBEC to deaminate cytosines to uracils. APOBEC also deaminates 5 mC and 5 hmC, making it impossible to differentiate between cytosine and its modified forms. In order to detect 5 mC and 5 hmC, this method also utilizes TET2 and an Oxidation Enhancer, which enzymatically modifies 5 mC and 5 hmC to forms that are not substrates for APOBEC. The TET2 enzyme converts 5 mC to 5 caC and the Oxidation Enhancer converts 5 hmC to 5 ghmC. Ultimately, cytosines are sequenced as thymines and 5 mC and 5 hmC are sequenced as cytosines, thereby protecting the integrity of the original 5 mC and 5 hmC sequence information.

In one embodiment, prior to step (a) the one or more nucleic acid analytes are introduced to an epigenetic modification-sensitive or epigenetic modification-dependent restriction endonuclease.

In one embodiment, the epigenetic modification-sensitive or epigenetic modification-dependent restriction endonuclease is McrBC.

In one embodiment, the epigenetic modification-sensitive or epigenetic modification-dependent restriction endonuclease is a member of the MspJI family.

In one embodiment, the endonuclease is AspBHI. In one embodiment, the endonuclease is FspEI.

In one embodiment, the endonuclease is LpnPI.

In one embodiment, the epigenetic modification-sensitive or epigenetic modification-dependent restriction endonuclease is a member of the PvuRts1I/AbaS family.

In one embodiment, the epigenetic modification-sensitive or epigenetic modification-dependent restriction endonuclease is a Type IIM endonuclease.

In one embodiment, the endonuclease is DpnI.

In one embodiment, the endonuclease is BisI.

In one embodiment, the epigenetic modification-sensitive or epigenetic modification-dependent restriction endonuclease is a Type IV endonuclease.

In one embodiment, the endonuclease is EcoKMcrBC.

In one embodiment, the endonuclease is SauUSI.

In one embodiment, the endonuclease is GmrSD.

In one embodiment, the epigenetic modification-sensitive or epigenetic modification-dependent restriction endonuclease is selected from the DpnII restriction endonuclease family.

In one embodiment, the endonuclease is DpnII.

In one embodiment, the endonuclease is DpnI.

In one embodiment, the epigenetic modification-sensitive or epigenetic modification-dependent restriction endonuclease is HpaI.

In one embodiment, the epigenetic modification-sensitive or epigenetic modification-dependent restriction endonuclease is HpaII.

In some embodiments, prior to step (a) the one or more nucleic acid analytes are introduced to a methylation-sensitive or methylation-dependent restriction endonuclease.

In some embodiments, prior to step (a) the one or more nucleic acid analytes are introduced to a methylation-sensitive or methylation-dependent restriction endonuclease followed by selective amplification of the target polynucleotide sequence containing the methylation status of interest through methylation-specific multiplex ligation-dependent probe amplification (MS-MLPA) of methylated DNA.

In some embodiments, prior to step (a) the population of methylated or unmethylated nucleic acid analytes is reduced prior to step (a).

In some embodiments, the reduction is carried out using methylated DNA immunoprecipitation (MeDIP).

In some embodiments, the reduction is carried out using methyl-binding proteins, such as MBD2b or the MBD2b/MBD3L1 complex.

It will be obvious to one skilled in the art that the present invention may be extended towards the detection of any epigenetic modification and is not limited to the detection of methylation status of target polynucleotide sequences. For example, the present invention could equally be adapted for the detection of other epigenetic modifications including hydroxymethylation—for example the hydroxylated form of 5 mC (5-hmC). This recently appreciated form of epigenetic modification is an important epigenetic marker which influences gene expression and is distinct from CpG methylation. Other epigenetic modifications appear on RNA such as methyl adenosine and can be detected by methods of the invention.

In some embodiments the method according to invention is where the epigenetic modification is methylation. In further embodiments the epigenetic modification is methylation at CpG islands or by hydroxymethylation at CpG islands.

In some embodiments the epigenetic modification is methylation of adenine in either RNA or DNA.

In some embodiments, each capture oligonucleotide further comprises a modification rendering it resistant to pyrophosphorolysis. In some embodiments, each capture oligonucleotide is made resistant to pyrophosphorolysis via mismatches at their 3′ ends. In some embodiments, each capture oligonucleotide is made resistant to pyrophosphorolysis via the presence of a 3′-blocking group. In some embodiments, each capture oligonucleotide is made resistant to pyrophosphorolysis via the presence of spacers or other internal modifications.

In some embodiments, one or more oligonucleotides of the current invention are rendered resistant to pyrophosphorolysis and/or exonuclease digestion by the presence of one or more quenchers.

In some embodiments, the method further comprises one or more wash steps to remove any unbound oligonucleotides.

In some embodiments, after the addition of a suitable washing buffer, the resulting reaction mixture is mixed.

In some embodiments, the resulting reaction mixture is mixed by vortexing.

In some embodiments, the resulting reaction mixture is mixed by the movement of one or more magnetic beads present in the mixture.

In some embodiments, each wash step comprises the use of a washing buffer comprising one or more of: Tris.HCl pH 7.5-8.0 5-20 mM, NaCl 0.4-2 M, EDTA 0.1-1 mM and/or Tween20 0-0.1%.

In some embodiments, the final step of the method is achieved by the combination of the second and/or third reaction mixtures with a fourth reaction mixture comprising:
at least one single-stranded primer oligonucleotide, deoxynucleotide triphosphates (dNTPs) and an amplification enzyme; or
reagents suitable for the hybridisation chain reaction (HCR); or
reagents suitable for the ligation chain reaction (LCR);
wherein the pyrophosphorolysis enzyme is optionally the same enzyme which performs amplification and thus optionally the fourth reaction mixture does not comprise an amplification enzyme.

In some embodiments, the deoxynucleotide triphosphates (dNTPs) are hot start dNTPs.

Hot start deoxynucleotide triphosphates (dNTPs) are dNTPs which are modified with a thermolabile protecting group at the 3′ terminus. The presence of this modification blocks DNA polymerase nucleotide incorporation until the nucleotide protecting group is removed using a heat activation step.

In an alternative embodiment, the fourth reaction mixture further comprises components for the hybridisation chain reaction (HCR).

In some embodiments, the fourth reaction mixture further comprises a ligation probe oligonucleotide C which has a 5′ phosphate, a splint oligonucleotide D which is complementary to the 3′ end of A₁ and the 5′ end of C, and the partially digested strand A₁ is ligated at the 3′ end to the 5′ end of C to form oligonucleotide A₂. In this embodiment, the fourth reaction mixture further comprises hairpin oligonucleotide 1 (HO1) and hairpin oligonucleotide 2 (HO2), each of which comprises a fluorophore and quencher such that when each oligonucleotide remains in a hairpin configuration the fluorophore and quencher are in contact with each other. HO1 is designed such that A₂ anneals to it, opening the ‘hairpin’ structure and separating the fluorophore from the quencher. The now ‘open’ HO1 is now able to anneal to HO2, opening the ‘hairpin’ structure and separating the fluorophore from the quencher.

In this embodiment, there are a plurality of hairpin oligonucleotides present such that the presence of one A₂ is able to cause a chain reaction of hairpin oligonucleotide opening resulting in the generation of a detectable fluorescent signal. This methodology is known in the literature as the Hybridisation Chain Reaction (HCR).

In some embodiments, the fluorophore of the fluorophore-quencher pair is selected from, but not limited to, dyes of the fluorescein family, the carboxyrhodamine family, the cyanine family, and the rhodamine family. Other families of dyes that can be used include, e.g., polyhalofluorescein-family dyes, hexachlorofluorescein-family dyes, coumarin-family dyes, oxazine-family dyes, thiazine-family dyes, squaraine-family dyes, chelated lanthanide-family dyes, the family of dyes available under the trade designation Alexa Fluor J, from Molecular Probes, the family of dyes available under the trade designation Atto from ATTO-TEC (Siegen, Germany) and the family of dyes available under the trade designation Bodipy J, from Invitrogen (Carlsbad, Calif.). Dyes of the fluorescein family include, e.g., 6-carboxyfluorescein (FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), 2′-chloro-7′-phenyl-1,4-dichloro-6-carboxyfluorescein (VIC), 6-carboxy-X-rhodamine (ROX), and 2′,4′,5′,7′-tetrachloro-5-carboxy-fluorescein (ZOE). Dyes of the carboxyrhodamine family include tetramethyl-6-carboxyrhodamine (TAMRA), tetrapropano-6-carboxyrhodamine (ROX), Texas Red, R110, and R6G. Dyes of the cyanine family include Cy2, Cy3, Cy3.5, Cy5, Cy5.5, and Cy7.

Fluorophores are readily available commercially from, for instance, Perkin-Elmer (Foster City, Calif.), Molecular Probes, Inc. (Eugene, Oreg.), and Amersham GE Healthcare (Piscataway, N.J.).

In some embodiments, the quencher of the fluorophore-quencher pair may be a fluorescent quencher or a non-fluorescent quencher. Fluorescent quenchers include, but are not limited to, TAMRA, ROX, DABCYL, DABSYL, cyanine dyes including nitrothiazole blue (NTB), anthraquinone, malachite green, nitrothiazole, and nitroimidazole compounds. Exemplary non-fluorescent quenchers that dissipate energy absorbed from a fluorophore include those available under the trade designation Black Hole™ from Biosearch Technologies, Inc. (Novato, Calif.), those available under the trade designation Eclipse™. Dark, from Epoch Biosciences (Bothell, Wash.), those available under the trade designation Qx1J, from Anaspec, Inc. (San Jose, Calif.), those available under the trade designations ZEN and TAO from Integrated DNA Technologies (Coralville, Iowa) and those available under the trade designation Iowa Black™ from Integrated DNA Technologies (Coralville, Iowa).

In some embodiments, the fluorophore of the fluorophore-quencher pair may be fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2-,2′-disulfonic acid derivatives.

In some embodiments, the fluorophore of the fluorophore-quencher pair may be LC-Red 640, LC-Red 705, Cy5, Cy5.5, Lissamine rhodamine B sulfonyl chloride, tetramethyl rhodamine isothiocyanate, rhodamine×isothiocyanate, erythrosine isothiocyanate, fluorescein, diethylenetriamine pentaacetate or other chelates of Lanthanide ions (e.g., Europium, or Terbium).

In some embodiments, the invention utilises fluorescently labelled oligonucleotides that are double quenched. The inclusion of a second, internal quencher shortens the distance between the dye and quencher and, in concert with the first quencher, provides greater overall dye quenching, lowering background and increasing signal detection. The second and first quenchers may be any of the quenchers previously described.

In an alternative embodiment, the fourth reaction mixture comprises a ligation probe oligonucleotide C which has a 5′ phosphate, a splint oligonucleotide D which is complementary to the 3′ end of A₁ and the 5′ end of C, and the partially digested strand A₁ is ligated at the 3′ end to the 5′ end of C to form oligonucleotide A₂.

In some embodiments, the 5′ and 3′ ends of A₁ are ligated together to form a circularised A₂.

In some embodiments, A₁ is circularised to form A₂ against a ligation probe oligonucleotide C.

In some embodiments, A₀ is circularised to form A₂, against a splint oligonucleotide D.

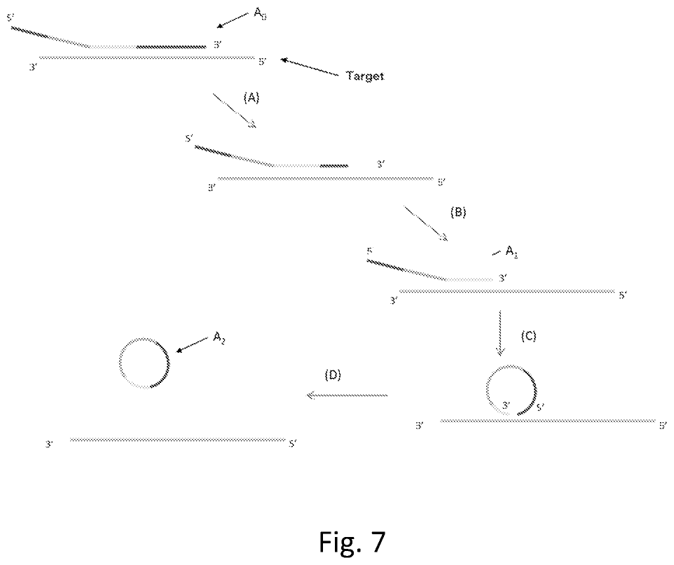

In some embodiments, A₁ is circularised to form A₂ against the target sequence. In this embodiment, the region of the target that is revealed by progressive digestion of A₀, in the 3′-5′ direction from the 3′ end of A₀ to form A₁, is complementary to the 5′ end of A₀/A₁. In this embodiment, a ligase may be used to ligate the 3′ and 5′ ends of A₁ to form a circularised oligonucleotide A₂. This is shown, for example in FIG. 7. In one embodiment, the 5′ end of A₀/A₁ is complementary to the target across a region that is 5-50 nucleotides in length. In one embodiment, it is 5-25 nucleotides in length. In one embodiment, it is 5-20 nucleotides in length.

In one embodiment, it is 5-15 nucleotides in length. In one embodiment, it is 5-12 nucleotides in length. In one embodiment, it is 5-10 nucleotides in length.

In some embodiments, A₁ is circularised to form A₂ as previously or subsequently, described.

In some embodiments, A₂ is formed from partially digested strand A₁ as previously, or subsequently, described.

In this embodiment, the fourth reaction mixture further comprises:
an oligonucleotide A comprising a substrate arm, a partial catalytic core and a sensor arm;
an oligonucleotide B comprising a substrate arm, a partial catalytic core and a sensor arm; and
a substrate comprising a fluorophore-quencher pair;
wherein the sensor arms of oligonucleotides A and B are complementary to flanking regions of A₂ such that in the presence of A₂ oligonucleotides A and B are combined to form a catalytically, multicomponent nucleic acid enzyme (MNAzyme). In this embodiment, the MNAzyme is formed only in the presence of A₂ and cleaves the substrate comprising a fluorophore-quencher pair such that a detectable fluorescent signal is generated.

In some embodiments, the fluorophore-quencher pair may be as described previously. In some embodiments, the first and fourth reaction mixtures are combined.

In some embodiments, the reaction mixtures of the invention are combined such that pyrophosphorolysis, ligation and the generation of a detectable fluorescent signal occurs without the addition of further reagents.

In an alternative embodiment, the fourth reaction mixture further comprises a partially double-stranded nucleic acid construct wherein:
one strand comprises at least one RNA base, at least one fluorophore and wherein a region of this strand is complementary to a region of A₂ and wherein this strand may be referred to as the ‘substrate’ strand;
the other stand comprises at least one quencher and wherein a region of this strand is complementary to a region of A₂ adjacent to that which the substrate strand is complementary to, such that in the presence of A₂ the partially double stranded nucleic acid construct becomes substantially more double-stranded;
wherein in the process of becoming substantially more double-stranded the substrate strand of the double-stranded nucleic acid construct is cut at the RNA base, resulting in fluorescence due to the at least one quencher of the ‘other’ strand no longer being in close enough proximity to that of the at least one fluorophore of the substrate strand.

In other words, the partially double stranded nucleic acid construct, in the presence of A₂, has a double-stranded portion which is greater in size.

In some embodiments, the fluorophore-quencher pair may be as described previously.

In some embodiments, further reagents such as suitable buffers and/or ions are present in the third reaction mixture.

In some embodiments, the fourth reaction mixture further comprises Mg²⁺ ions.

In some embodiments, the fourth reaction mixture further comprises Zn²⁺ ions.

In some embodiments, the fourth reaction mixture further comprises X²⁺ ions, wherein X is a metal.

In some embodiments, the fourth reaction mixture further comprises one or more X²⁺ ions, wherein X is a metal.

In an alternative embodiment, the fourth reaction mixture further comprises reagents for the ligase chain reaction (LCR).

In some embodiments, the fourth reaction mixture comprises
a. one or more ligases; and
b. two or more LCR probe oligonucleotides that are complementary to adjacent sequences on A₂, wherein when the probes are successfully annealed to A₂ the 5′ phosphate of one LCR probe is directly adjacent to the 3′OH of the other LCR probe;
c. In some embodiments, in the presence of A₂ the two LCR probes will successfully anneal to A₂ and be ligated together to form one oligonucleotide molecule which subsequently acts as a new target for second-round covalent ligation, leading to geometric amplification of the target of interest, in this case A₂. The ligated products, or amplicons, are complementary to A₂ and function as targets in the next cycle of amplification. Thus, exponential amplification of the specific target DNA sequences is achieved through repeated cycles of denaturation, hybridization, and ligation in the presence of excess LCR probes. From this, the presence of A₂ and hence the target polynucleotide sequence is inferred.

In some embodiments, in the presence of A₂ the two PCR probes will successfully anneal to A₂ and be ligated together to form one oligonucleotide molecule which then acts as a new target for second-round covalent ligation, leading to geometric amplification of the target of interest, in this case A₂, which is then detected.

In some embodiments, the ligated oligonucleotide molecule is detected in real time using an intercalating dye.

In some embodiments, the ligated oligonucleotide molecule is detected using gel electrophoresis.

The person skilled in the art will appreciate there are numerous techniques which would allow the detection of the ligated oligonucleotide molecule.

In some embodiments, the deoxynucleotide triphosphates (dNTPs) are hot start dNTPs.

In some embodiments, the one or more ligases are thermostable.

In some embodiments, the one or more ligases are naturally occurring.

In another embodiment, the one or more ligases are engineered.

In some embodiments, the one or more ligases are selected from any ligase disclosed previously or subsequently.

In some embodiments, the one or more polymerases are thermostable.

In some embodiments, the one or more polymerases are selected from any polymerase disclosed previously or subsequently.

In some embodiments, the one or more polymerases are naturally occurring.

In another embodiment, the one or more polymerases are engineered.

In some embodiments, the one or more polymerases are the same as that used for the pyrophosphorolysis.

In some embodiments, the fourth reaction mixture further comprises a pyrophosphorolyising enzyme in addition to one or more polymerases.

In some embodiments, one or more enzymes of the current invention are hot start enzymes.

In some embodiments, one or more enzymes of the current invention are thermostable.

In an alternate embodiment, the fourth reaction mixture comprises:
a. a splint oligonucleotide, comprising a fluorophore-quencher pair, which is complementary to A₂;
b. a double strand specific DNA digestion enzyme;
wherein, in the presence of A₂, the splint oligonucleotide is digested such that the fluorophore-quencher pair are separated and a fluorescent signal, and hence the presence of A₂, is detectable.

In some embodiments, the fluorophore-quencher pair may be as described previously. In some embodiments, the double strand specific DNA digestion enzyme is an exonuclease. In another embodiment, it is a polymerase with proofreading activity. In another embodiment, the fourth reaction mixture comprises a mixture of one or more of: an exonuclease or a polymerase with proofreading activity.

In some embodiments, the double strand specific DNA digestion enzyme is a hot start enzyme.

In some embodiments, the double strand specific DNA digestion enzyme has reduced activity at the temperature at which the pyrophosphorolysis reaction of the method takes place.

In some embodiments, the double strand specific DNA digestion enzyme has no activity at the temperature at which the pyrophosphorolysis reaction of the method takes place.

In some embodiments, prior to step (a) of the method, any RNA template present in the sample is transcribed into DNA.

In some embodiments, this is achieved via the use of a reverse transcriptase and appropriate nucleotides.

In some embodiments, any RNA present in the sample is transcribed into DNA at the same time as any pre-amplification via PCR of nucleic acids present in the sample.

In some embodiments, the transcription of any RNA present in the sample into DNA occurs in a separate step as any pre-amplification via PCR of nucleic acids present in the sample.

In some embodiments of any of the previously, or subsequently, described methods, RNA present in the sample is not transcribed into DNA. In such an embodiment, A₀ undergoes pyrophosphorolysis against an RNA sequence to form partially digested strand A₁ and the method then proceeds as previously, or subsequently, described.

In some embodiments of any of the previously, or subsequently, described methods, one or more reaction mixtures may be combined.

In some embodiments, either:
A₁ is circularised through ligation of its 3′ and 5′ ends to create A₂; or
the second, third or fourth reaction mixtures further comprises a ligation probe oligonucleotide C and that the ligation A₁ undergoes to form A₂ is ligation of the 3′ end of A₁ to the 5′ end of C.

In some embodiments, the ligation of A₁ occurs:
during step (c);
during step (d); or
between steps (c) and (d).

In some embodiments, the oligonucleotide C further comprises a 3′ or internal modification protecting it from 3′-5′ exonuclease digestion.

In some embodiments, the oligonucleotide C further comprises a 5′ modification protecting it from 5′-3′ exonuclease digestion.

In some embodiments, the second reaction mixture or the third reaction mixture to which the second reaction mixture is combined further comprises a splint oligonucleotide D.

In some embodiments, D comprises an oligonucleotide region complementary to the 3′ end of A₁ and a region complementary to either the 5′ end of oligonucleotide C or to the 5′ end of A₁.

In some embodiments, D is unable to undergo extension against A₁ by virtue of either a 3′ modification or through a mismatch between the 3′ end of D and the corresponding region of A₁.

In some embodiments, the method further comprises a two-step amplification performed between steps (c) and (d).

In some embodiments, the reaction volume is split into two or more separate volumes prior to the second amplification.

The person skilled in the art will understand there are a plethora of 3′ modifications which may be used to prevent extension.

In some embodiments, the second reaction mixture further comprises a 5′-3′ exonuclease and wherein the 5′ end of A₀ is rendered resistant to 5′-3′ exonuclease digestion.

In some embodiments, prior to or during the final step, the products of the previous step are treated with a pyrophosphatase.

In some embodiments, prior to or during the final step, the products of the previous step are treated with an exonuclease.

In some embodiments, detection is achieved using one or more oligonucleotide fluorescent binding dyes or molecular probes.

In some embodiments, an increase in signal over time resulting from the generation of amplicons of A₂ is used to infer the concentration of the target sequence in the analyte.

In some embodiments, multiple probes A₀ and multiple capture oligonucleotides B₀ are employed, wherein each A₀ is selective for a different target sequence and includes an identification region, wherein each B₀ comprises a region complementary to a region adjacent to a target sequence and further characterised in that the amplicons of A₂ include the identification region and therefore the target sequences present in the analyte, are inferred through the detection of the identification region(s).

In some embodiments, detection of the identification regions(s) is carried out using molecular probes or through sequencing.

In some embodiments, the final step of the method further comprises the steps of:
i. labelling the products of the previous step using one or more oligonucleotide fluorescent binding dyes or molecular probes;
ii. measuring the fluorescent signal of the products;
iii. exposing the products to a set of denaturing conditions; and
identifying the polynucleotide target sequence in the analyte by monitoring changes in the fluorescent signal of the products during exposure to the denaturing conditions.

In some embodiments, the one or more nucleic acid analytes are split into multiple reaction volumes, each volume having a one or more probe oligonucleotide A₀, introduced to detect different target sequences.

In some embodiments, the one or more nucleic acid analytes are split into multiple reaction volumes, each volume having one or more probe oligonucleotide A₀, and one or more capture oligonucleotide B₀, introduced to detect different target sequences.

In some embodiments, the different probes A₀ comprise common priming sites, allowing a single primer or single set of primers to be used for amplification of a region of A₂.

In an alternative embodiment, the fourth reaction mixture further comprise one or more partially double stranded DNA constructs wherein each construct contains one or more fluorophores and one or more quenchers. In some embodiments, when the construct is partially double-stranded the one or more fluorophores and one or more quenchers are located in close enough proximity to each other such that sufficient quenching of the one or more fluorophores occurs.

In some embodiments, the construct is one strand of DNA with a self-complementary region that is looped back on itself.

In some embodiments, the construct comprises one primer of a primer pair.

In some embodiments, the fourth reaction mixture further comprises the other primer of a primer pair.

In some embodiments, a portion of the single stranded section of the construct hybridises to A₂ and is extended against it by a DNA polymerase. In some embodiments, the other primer of the primer pair then hybridises to the extended construct. This primer is then extended against the construct, displacing the self-complementary region. Thus, the one or more fluorophores and one or more dyes are separated sufficiently for a fluorescent signal to be detected, indicating the presence of A₂ in the reaction mixture.

In such an embodiment the construct may be known as a Sunrise Primer.

In some embodiments, the construct comprises two separate DNA strands.

In some embodiments, a portion of the single stranded section of the construct hybridises to A₂ and is extended against it by a DNA polymerase. In some embodiments, the other primer of the primer pair then hybridises to the extended construct. This primer is then extended against the construct, in the direction of the double stranded section, displacing the shorter of the DNA strands and thus the one or more fluorophores and one or more dyes are separated sufficiently for a fluorescent signal to be detected, indicating the presence of A₂ in the reaction mixture.

In such an embodiment the construct may be known as a Molecular Zipper.

The person skilled in the art will appreciate that for both the Sunrise Primer and Molecular Zipper it is possible for the one or more fluorophores and the one or more quencher pairs to be located at various positions within each respective construct. The key feature is that each pair is located in sufficient proximity to one another that in the absence of A₂, i.e. when no extension and strand displacement has occurred, no fluorescent signal is emitted.

In some embodiments, the method further comprises the transcription of any RNA present in a sample to DNA. RNA is transcribed to DNA in the presence of a suitable enzyme and suitable deoxynucleotide triphosphates (dNTPs). One such enzyme is reverse transcriptase (RT).

This conversion may occur prior to step (a).

This conversion may occur during step (a), in such embodiments the first reaction mixture further comprises an enzyme, such as RT, which transcribes RNA to DNA. The first reaction mixture may further comprise appropriate dNTPs and one or more primers.

In some embodiments according to any of the previously, or subsequently, described methods, RNA present in the sample is not transcribed to DNA. In such an embodiment, A₀ undergoes pyrophosphorolysis against an RNA sequence to form partially digested strand A₁ and the method then proceeds as previously, or subsequently, described.

In some embodiments of any of the previously, or subsequently, described methods, one or more reaction mixtures may be combined. According to the present invention, there are further provided methods of detecting a target polynucleotide sequence in a given nucleic acid analyte.

The analytes to which the various methods of the invention can be applied may be prepared from the biological sample mentioned above by a series of preliminary steps designed to amplify the analyte and separate it from the background genomic DNA which is typically present in significant excess.

In some embodiments, the target polynucleotide sequence in the analyte will be a gene or chromosomal region within the DNA or RNA of a cancerous tumour cell and will be characterised by the presence of one or more mutations; for example in the form of one or more single nucleotide polymorphisms (SNPs). Thus the invention will be useful in the monitoring of and/or treatment for disease recurrence. Patients who have been declared free of disease following treatment may be monitored over time to detect the recurrence of disease. This needs to be done non-invasively and requires sensitive detection of target sequences from blood samples. Similarly, for some cancers there are residual cancer cells that remain in a patient after treatment. Monitoring of the levels of these cells (or cell free DNA) present in the patient's blood, using the current invention, allows detection of recurrence of disease or failure of current therapy and the need to switch to an alternative.

In some embodiments, detection of the target polynucleotide sequence will allow repeated testing of patient samples during treatment of disease to allow early detection of developed resistance to therapy. For example, epidermal growth factor receptor (EGFR) inhibitors, such as gefitinib, erlotinib, are commonly used as first line treatments for non-small cell lung cancer (NSCLC). During treatment the tumour will often develop mutations in the EGFR gene (e.g T790M, C797S) which confer resistance to the treatment. Early detection of these mutations allows transfer of the patient onto alternative therapies.

In some embodiments, the target polynucleotide sequence in the analyte will be a gene or chromosomal region within the DNA or RNA of fetal origin and will be characterised by the presence of one or more mutations; for example in the form of one or more single nucleotide polymorphisms (SNPs). Thus, the invention may be used to detect mutations at very low allele fractions, at an earlier stage of pregnancy than other available testing techniques.

In another embodiment, the target polynucleotide sequence may be a gene or genomic region derived from an otherwise healthy individual but the genetic information obtained may assist in generating valuable companion diagnostic information allowing medical or therapeutic conclusions to be drawn across one or more defined groups within the human population.

In yet another embodiment, the target polynucleotide sequence may be characteristic of an infectious disease, or of resistance of an infectious disease to treatment with certain therapies; for example a polynucleotide sequence characteristic of a gene or chromosomal region of a bacterium or a virus, or a mutation therein conferring resistance to therapy.

In some embodiments, the target polynucleotide sequence may be characteristic of donor DNA. When a transplanted organ is rejected by the patient, the DNA from this organ is shed into the patient's bloodstream. Early detection of this DNA would allow early detection of rejection. This could be achieved using custom panels of donor-specific markers, or by using panels of variants known to be common in the population, some of which will be present in the donor and some in the recipient. Routine monitoring of organ recipients over time is thus enabled by the claimed method.

The success of organ transplantation can depend on the overall level of cumulative injury to the organ caused by several events in the donor. This includes age, lifestyle, ischemia/reperfusion injury (IRI) and immune response in the recipient. Research has shown that IRI causes epigenetic changes in the donor organ. The promoter region of the C3 gene becomes demethylated in the kidney, which is associated with chronic nephropathy post-transplantation. DNA methylation is a major contributor to a balanced immune response toward a graft as it regulates the function of cells of the immune system. Thus, detection of the methylation status of particular DNA sequences can allow identification of patients at risk for post-transplant complications.

In yet another embodiment, various versions of the method using different combinations of probes (see below) are employed in parallel so that the analyte can be simultaneously screened for multiple target sequences; for example sources of cancer, cancer indicators or multiple sources of infection. In this approach, the amplified products obtained by parallel application of the method are contacted with a detection panel comprised of one or more oligonucleotide binding dyes or sequence specific molecular probes such as a molecular beacon, hairpin probe or the like. Thus, in another aspect of the invention there is provided the use of at least one probe and optionally one ligation oligonucleotide in combination with one or more chemical and biological probes selective for the target polynucleotide sequences or with the use of sequencing to identify the amplified probe regions.

In some embodiments, the single-stranded probe oligonucleotide A₀ comprises a priming region and a 3′ end which is complementary to the target polynucleotide sequence to be detected. By this means, a first intermediate product is created which is at least partially double-stranded. In some embodiments, this step is carried out in the presence of excess A₀ and in an aqueous medium containing the analyte and any other nucleic acid molecules.

During step (c), the double-stranded region of the first intermediate product is pyrophosphorolysed in the 3′-5′ direction from the 3′ end of its A₀ strand. As a consequence, the A₀ strand is progressively digested to create a partially digested strand; hereinafter referred to as A₁. Where the probe oligonucleotide erroneously hybridises with a non-target sequence, the pyrophosphorolysis reaction will stop at any mismatches, preventing subsequent steps of the method from proceeding. In another embodiment, this digestion continues until A₁ lacks sufficient complementarity with the analyte or a target region therein to form a stable duplex. At this point, the various strands then separate by melting, thereby producing A₁ in single-stranded form. Under typical pyrophosphorolysis conditions, this separation occurs when there are between 6 and 20 complementary nucleotides between the analyte and A₀.

In another embodiment, the digestion continues until A₁ lacks sufficient complementarity with the analyte or target region therein for the pyrophosphorolyising enzyme to bind or for the pyrophosphorolyising reaction to continue. This typically occurs when there are between 6 and 20 complementary nucleotides remaining between the analyte and probe. In some embodiments, this occurs when there are between 6 and 40 complementary nucleotides remaining.

In another embodiment, in which a splint oligonucleotide D having complementarity to the 5′ and 3′ ends of A₁ is employed (see below), the digestion continues until the length of complementarity between A₁ and the target is reduced to the point at which it is energetically favourable for oligo D to displace the analyte molecule from A₁. This typically occurs when the region of complementarity between A₁ and the analyte molecule is of similar or shorter length than the region of complementarity between oligo D and the 3′ end of A₁, but may also occur when the complementarity between A₁ and the analyte molecule is longer than this due to the favourability of intra-molecular hybridisation of the oligo D, which may already be hybridised to the 5′ end of A₁.

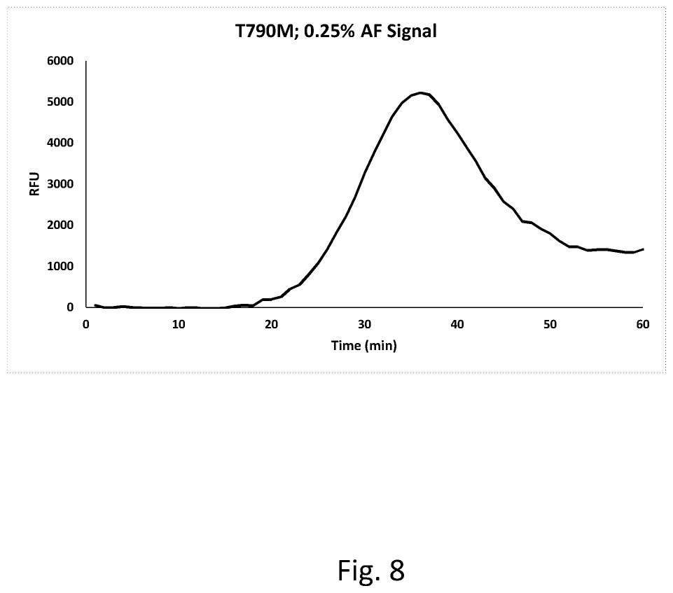

In another embodiment, in which the ligation of A₁ is performed using the analyte molecule as a splint (see FIG. 8), the digestion continues until the 5′ end of A₁ is able to hybridise to the analyte molecule such that the 3′ and 5′ ends of A₁ are neighbouring and are separated only by a nick, at which point they are ligated together by the ligase and digestion is no longer able to proceed.

Suitably, pyrophosphorolysis is carried out in the reaction medium at a temperature in the range 20 to 90° C. in the presence of at least a polymerase exhibiting pyrophosphorolysis activity and a source of pyrophosphate ion. Further information about the pyrophosphorolysis reaction as applied to the digestion of polynucleotides can be found for example in J. Biol. Chem. 244 (1969) pp. 3019-3028 or our earlier patent application.

In some embodiments, the pyrophosphorolysis step is driven by the presence of a source of excess polypyrophosphate, suitable sources including those compounds containing 3 or more phosphorous atoms.

In some embodiments, the second reaction mixture comprises a source of excess polypyrophosphate.

In some embodiments, the pyrophosphorolysis step is driven by the presence of a source of excess modified pyrophosphate. Suitable modified pyrophosphates include those with other atoms or groups substituted in place of the bridging oxygen, or pyrophosphate (or polypyrophosphate) with substitutions or modifying groups on the other oxygens. The person skilled in the art will understand that there are many such examples of modified pyrophosphate which would be suitable for use in the current invention, a non-limiting selection of which are:

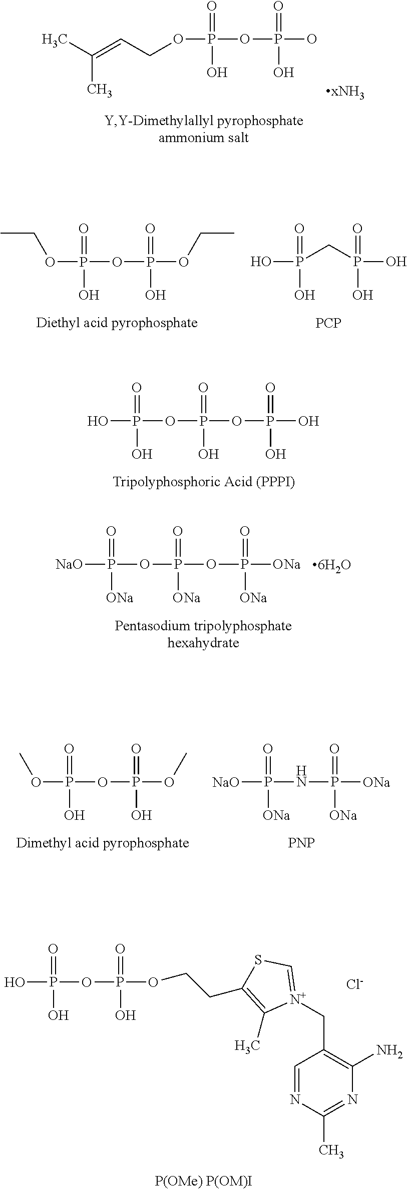

In some embodiments, the second reaction mixture comprises a source of excess modified polypyrophosphate.

In one preferred embodiment, the source of pyrophosphate ion is PNP, PCP or Tripolyphoshoric Acid (PPPi).

Further, but not limiting, examples of sources of pyrophosphate ion for use in the pyrophosphorolysis step (c) may be found in WO2014/165210 and WO00/49180.

In some embodiments, the source of excess modified pyrophosphate can be represented as Y—H wherein Y corresponds to the general formula (X—O)₂P(═B)—(Z—P(═B)(O—X))ₙ— wherein n is an integer from 1 to 4; each Z— is selected independently from —O—, —NH— or —CH₂—; each B is independently either O or S; the X groups are independently selected from —H, —Na, —K, alkyl, alkenyl, or a heterocyclic group with the proviso that when both Z and B correspond to —O— and when n is 1 at least one X group is not H.

In some embodiments, Y corresponds to the general formula (X—O)₂P(═B)—(Z—P(═B)(O—X))ₙ— wherein n is 1, 2, 3 or 4. In another embodiment, the Y group corresponds to the general formula (X—O)₂P(═O)—Z—P(═O)(O—H)— wherein one of the X groups is —H. In yet another preferred embodiment, Y corresponds to the general formula (X—O)₂P(═O)—Z—P(═O)(O—X)— wherein at least one of the X groups is selected from methyl, ethyl, allyl or dimethylallyl.

In an alternative embodiment, Y corresponds to either of the general formulae (H—O)₂P(═O)—Z—P(═O)(O—H)— wherein Z is either —NH— or —CH₂— or (X—O)₂P(═O)—Z—P(═O)(O—X)— wherein the X groups are all either— Na or —K and Z is either —NH— or —CH₂—.

In another embodiment, Y corresponds to the general formula (H—O)₂P(═B)—O—P(═B)(O—H)— wherein each B group is independently either O or S, with at least one being S.

Specific examples of preferred embodiments of Y include those of the formula (X1—O)(HO)P(═O)—Z—P(═O)(O—X2) wherein Z is O, NH or CH₂ and (a) X1 is γ,γ-dimethylallyl, and X2 is —H; or (b) X1 and X2 are both methyl; or (c) X1 and X2 are both ethyl; or (d) X1 is methyl and X2 is ethyl or vice versa.

In some embodiments, where molecular probes are to be used for detection, the probe oligonucleotide A₀ is configured to include an oligonucleotide identification region on the 5′ side of the region complementary to the target sequence, and the molecular probes employed are designed to anneal to this identification region. In some embodiments, only the 3′ region of A₀ is able to anneal to the target; i.e. any other regions lack sufficient complementarity with the analyte for a stable duplex to exist at the temperature at which the pyrophosphorolysis step is carried out. Here and throughout, by the term ‘sufficient complementarity’ is meant that, to the extent that a given region has complementarity with a given region on the analyte, the region of complementarity is more than 10 nucleotides long.

In a further aspect of the methods of the invention there is provided alternate embodiments in which the phosphorolysis step of any previous embodiment is replaced with an exonuclease digestion step using a double-strand specific exonuclease. The person skilled in the art will understand that double-strand specific exonucleases include those that read in the 3′-5′ direction, such as ExoIII, and those that read in the 5′-3′ direction, such as Lambda Exo, amongst many others.

In some embodiments of the invention wherein the exonuclease digestion step utilises a double strand-specific 5′-3′ exonuclease, it is the 5′ end of A₀ that is complementary to the target analyte and the common priming sequence and blocking group are located on the 3′ side of the region complementary to the target. In a further embodiment, where molecular probes are to be used for detection the probe oligonucleotide A₀ is configured to include an oligonucleotide identification region on the 3′ side of the region complementary to the target sequence, and the molecular probes employed are designed to anneal to this identification region.

In embodiments of the invention wherein the exonuclease digestion step utilises a double strand-specific 5′-3′ exonuclease, an exonuclease having 3′ to 5′ exonucleolytic activity can optionally be added to the second reaction mixture for the purpose of digesting any other nucleic acid molecules present whilst leaving A₀ and any material comprising partially digested strand A₁ intact. Suitably, this resistance to exonucleolysis is achieved as described elsewhere in this application.

In one preferable embodiment of the invention, the 5′ end of A₀ or an internal site on the 5′ side of the priming region is rendered resistant to exonucleolysis. By this means and after, or simultaneously with, the pyrophosphorolysis step, an exonuclease having 5′-3′ exonucleolytic activity can optionally be added to the reaction medium for the purpose of digesting any other nucleic acid molecules present whilst leaving A₀ and any material comprising the partially digested strand A₁ intact. Suitably, this resistance to exonucleolysis is achieved by introducing one or more blocking groups into the oligonucleotide A₀ at the required point. In some embodiments, these blocking groups may be selected from phosphorothioate linkages and other backbone modifications commonly used in the art, C3 spacers, phosphate groups, modified bases and the like.

The probe sequence may comprise naturally occurring bases including A, G, C or T. The C bases may be methylated or unmethylated. The bases may be bases other than A, G, C and T. For example the bases may include ‘wobble’ bases such as hypoxanthine or 5-nitro indole. Such wobble bases may be particularly useful in disrupting strands which have a high level of secondary structure such as that caused by high levels of G, in which G quadruplex structures may arise.

In some embodiments, the identification region will comprise or have embedded within a barcoding region which has a unique sequence and is adapted to be indirectly identified using a sequence-specific molecular probe applied to the amplified components A₂ or directly by the sequencing of these components. Examples of molecular probes which may be used include, but are not limited to, molecular beacons, TaqMan© probes, Scorpion® probes and the like.

In some embodiments the A₂ strand or a desired region thereof is caused to undergo amplification so that multiple, typically many millions, of copies are made. This is achieved by priming a region of A₂ and subsequently any amplicons derived therefrom with single-stranded primer oligonucleotides, provided for example in the form of a forward/reverse or sense/antisense pair, which can anneal to a complementary region thereon. The primed strand then becomes the point of origin for amplification. Amplification methods include, but are not limited to, thermal cycling and isothermal methods such as the polymerase chain reaction, recombinase polymerase amplification and rolling circle amplification; the last of these being applicable when A₂ is circularised. By any of these means, many amplicon copies of a region of A₂ and in some instances its sequence complement can be rapidly created. The exact methodologies for performing any of these amplification methods will be well-known to one of ordinary skill and the exact conditions and temperature regimes employed are readily available in the general literature to which the reader is directed. Specifically, in the case of the polymerase chain reaction (PCR), the methodology generally comprises extending the primer oligonucleotide against the A₂ strand in the 5′-3′ direction using a polymerase and a source of the various single nucleoside triphosphates until a complementary strand is produced; dehybridising the double-stranded product produced to regenerate the A₂ strand and the complementary strand; re-priming the A₂ strand and any of its amplicons and thereafter repeating these extension/dehybridisation/repriming steps multiple times to build-up a concentration of A₂ amplicons to a level where they can be reliably detected.

In some embodiments, the second reaction mixture further comprises a ligation probe oligonucleotide C, and the partially digested strand A₁ is ligated at the 3′ end to the 5′ end of C, while in another embodiment, A₁ is circularised through ligation of its 3′ and 5′ ends; in each case to create an oligonucleotide A₂.

In some embodiments, the ligation of A₁ occurs:
during step (c); or
during step (d); or
between steps (c) and (d).

In some embodiments, A₁ is optionally extended in 5′-3′ direction prior to ligation.

In some embodiments, this optional extension and the ligation are performed against the target oligonucleotide, while in another embodiment they are performed through addition of a further splint oligonucleotide D to which A₁ anneals prior to extension and/or ligation. In some embodiments, D comprises an oligonucleotide region complementary to the 3′ end of A₁ and a region complementary to either the 5′ end of oligonucleotide C or to the 5′ end of A₁. In another embodiment, D is unable to extend against A₁ by virtue of either a 3′-end modification or through a nucleotide mismatch between the 3′ end of D and the corresponding region of A₁.

In some embodiments, the ligation probe C has a 5′ region complementary to at least part of a 5′ end region of a splint oligonucleotide D or to the target oligonucleotide. By such means, a second intermediate product is formed in which the A₂ strand is comprised of A₁, C and optionally an intermediate region formed by extension of A₁ in the 5′-3′ direction to meet the 5′ end of C. In such an embodiment, if primers employed in step (d) they are chosen to amplify at least a region of A₂ including the site at which ligation of the A₁ to C has occurred. In this embodiment, we have found that it is advantageous to include a 3′ blocking group on C so that a 3′-5′ exonuclease can be used to digest any non-ligated A₁ prior to amplification and/or detection.

In some embodiments, the second reaction mixture further comprises a phosphatase or phosphohydrolase to remove by hydrolysis the nucleoside triphosphates produced by the pyrophosphorolysis reaction thereby ensuring that the pyrophosphorolysis reaction can continue and does not become out-competed by the forward polymerisation reaction.

In some embodiments, prior to or during step (d) the products of the previous step are treated with a pyrophosphatase to hydrolyse the pyrophosphate ion, preventing further pyrophosphorolysis from occurring and favouring the forward polymerisation reaction. In some embodiments, prior to or during step (d) the products of the previous step are treated with an exonuclease.

In some embodiments, the enzyme which performs pyrophosphorolysis of A₀ to form partially digested strand A₁ also amplifies A₂. The person skilled in the art will appreciate there exist many such enzymes.

The oligonucleotides A₂ are detected and the information obtained is used to infer whether the polynucleotide target sequence is present or absent in the original analyte and/or a property associated therewith. For example, by this means a target sequence characteristic of a cancerous tumour cell may be detected with reference to specific SNPs being looked for. As a further example, a target sequence characteristic of a cancerous tumour cell may be detected with reference to specific methylation sites being looked for.

In another embodiment, a target sequence characteristic of the genome of a virus of bacterium (including new mutations thereof) may be detected. Many methods of detecting amplicons or identification regions of A₂ can be employed including for example an oligonucleotide binding dye, a sequence-specific molecular probe such as fluorescently-labelled molecular beacon or hairpin probe. Alternatively, direct sequencing of A₂ of the amplicons thereof can be performed using one of the direct sequencing methods employed or reported in the art. Where oligonucleotide binding dyes, fluorescently labelled beacons or probes are employed it is convenient to detect the resulting signal using an arrangement comprising a source of stimulating electromagnetic radiation (laser, LED, lamp etc.) and a photodetector arranged to detect emitted fluorescent light and to generate therefrom a signal comprising a data stream which can be analysed by a microprocessor or a computer using specifically-designed algorithms.

In some embodiments, detection is achieved using one or more oligonucleotide fluorescent binding dyes or molecular probes. In such embodiments, an increase in signal over time resulting from the generation of amplicons of A₂ is used to infer the concentration of the target sequence in the analyte. In some embodiments that the final step of the method further comprises the steps of:
i. labelling the products of step (b) using one or more oligonucleotide fluorescent binding dyes or molecular probes;
ii. measuring the fluorescent signal of the products;
iii. exposing the products to a set of denaturing conditions; and
identifying the polynucleotide target sequence in the analyte by monitoring changes in the fluorescent signal of the products during exposure to the denaturing conditions.

In some embodiments, multiple probes A₀ and multiple capture oligonucleotides B₀ are employed, wherein each A₀ is selective for a different target sequence and includes an identification region, wherein each B₀ comprises a region complementary to a region adjacent to a target sequence and further characterised in that the resulting oligonucleotides A₂ include identification regions, and therefore the target sequences present in the analyte are inferred through the detection of these identification region(s).

In some embodiments, the one or more nucleic acid analytes are split into multiple reaction volumes, each volume having a one or more probe oligonucleotide A₀, and one or more capture oligonucleotide B₀, introduced to detect different target sequences.

In some embodiments, wherein different probes A₀ are used the different probes A₀ comprise one or more common priming sites, allowing a single primer or single set of primers to be used for amplification of A₂ or a region thereof.

In another aspect of the invention, there is provided a method of identifying a target polynucleotide sequence in a given nucleic acid analyte characterised by the steps of any previous embodiment of the invention wherein the multiple copies of A₂, or a region of A₂, are labelled using one or more oligonucleotide fluorescent binding dyes or molecular probes. The fluorescent signal of these multiple copies is measured and the multiple copies are exposed to a set of denaturing conditions. The target polynucleotide sequence is then identified by monitoring a change in the fluorescent signal of the multiple copies during exposure to the denaturing conditions.

In some embodiments, the denaturing conditions may be provided by varying the temperature e.g. increasing the temperature to a point where the double strand begins to dissociate. Additionally or alternatively, the denaturing conditions may also be provided by varying the pH such that the conditions are acidic or alkaline, or by adding in additives or agents such as a strong acid or base, a concentrated inorganic salt or organic solvent e.g. alcohol.

In another aspect of the invention, there is provided the use of the methods described above to screen mammalian subjects, especially human patients, for the presence of infectious diseases, cancer or for the purpose of generating companion diagnostic information.

In a further aspect of the invention, there is provided control probes for use in the methods as described above. Embodiments of the current invention include those wherein the presence of a specific target sequence, or sequences, is elucidated by the generation of a fluorescent signal. In such embodiments, there may inevitably be a level of signal generated from non-target DNA present in the sample. For a given sample, this background signal has a later onset than the ‘true’ signal, but this onset may vary between samples. Accurate detection of the presence of low concentrations of target sequence, or sequences, thus relies on knowledge of what signal is expected in its absence. For contrived samples references are available, but for true ‘blind’ samples from patients this is not the case. The control probes (E₀) are utilised to determine the expected background signal profile for each assay probe. The control probe targets a sequence not expected to be present in the sample and the signal generated from this probe can then be used to infer the expected rate of signal generation from the sample in the absence of target sequence.

Thus there is provided a method of detecting a target polynucleotide sequence in a given nucleic acid analyte according to any of the previously described methods characterised by the steps of:
a. either subsequently or concurrently repeating the steps of the methods using a second single-stranded probe oligonucleotide E₀ having a 3′ end region at least partially mismatched to the target sequence, either using a separate aliquot of the sample or in the same aliquot and using a second detection channel;
b. inferring the background signal expected to be generated from A₀ in the absence of any target analyte in the sample; and
c. through comparison of the expected background signal inferred in (a) with the actual signal observed in the presence of the target analyte inferring the presence or absence of the polynucleotide target sequence in the analyte.

In some embodiments, the control probe (E₀) and A₀ are added to separate portions of the sample while in another embodiment the E₀ and A₀ are added to the same portion of the sample and different detection channels (e.g. different colour dyes) used to measure their respective signals. The signal generated by E₀ may then be utilised to infer and correct for the background signal expected to be generated by A₀ in the absence of the polynucleotide target sequence in the sample. For example, a correction of the background signal may involve the subtraction of the signal observed from E₀ from that observed from A₀, or through the calibration of the signal observed from A₀ using a calibration curve of the relative signals generated by A₀ and E₀ under varying conditions.

In some embodiments, a single E₀ can be used to calibrate all of the assay probes which may be produced.

In some embodiments, a separate E₀ may be used to calibrate each amplicon of the sample DNA generated in an initial amplification step. Each amplicon may contain multiple mutations/target sequences of interest, but a single E₀ will be sufficient to calibrate all of the assay probes against a single amplicon.

In a further embodiment, a separate E₀ may be used for each target sequence. For example, if a C>T mutation is being targeted, an E₀ could be designed that targets a C>G mutation in the same site that is not known to occur in patients. The signal profile generated by E₀ under various conditions can be assessed in calibration reactions and these data used to infer the signal expected from the assay probe targeting the C>T variant when this variant is not present.

The specificity of the methods of the current invention may be improved by the introduction of blocking oligonucleotides. For example a blocking oligo nucleotide can be introduced so as to hybridise to at least a portion of wild-type DNA, promoting annealing of A₀ only to the target polynucleotide sequences and not the wildtype. Alternatively or additionally, blocking oligonucleotides can be used to improve the specificity of the polymerase chain reaction (PCR) to prevent amplification of any wild type sequence present. The general technique used is to design an oligonucleotide that anneals between the PCR primers and is not able to be displaced or digested by the PCR polymerase. The oligonucleotide is designed to anneal to the non-target (usually healthy) sequence, while being mismatched (often by a single base) to the target (mutant) sequence. This mismatch results in a different melting temperature against the two sequences, the oligonucleotide being designed to remain annealed to the non-target sequence at the PCR extension temperature while dissociating from the target sequence.

The blocking oligonucleotides may often have modifications to prevent its digestion by the exonuclease activity of the PCR polymerase, or to enhance the melting temperature differential between the target and non-target sequence.

The incorporation of a locked nucleic acid (LNA) or other melting temperature altering modification into a blocking oligonucleotide can significantly increase the differential in melting temperature of the oligonucleotide against target and non-target sequences.

Thus there is provided an embodiment of the invention wherein blocking oligonucleotides are used. The blocking oligonucleotides must be resistant to the pyrophosphorolysing (PPL) reaction to ensure they are not digested or displaced. This can be achieved in a number of different ways, for example via mismatches at their 3′ ends or through modifications such as phosphorothioate bonds or spacers.

In such embodiments or an aspect of the present invention where blocking oligonucleotides are used, the method of detecting a target polynucleotide sequence in a given nucleic acid analyte is characterised by annealing single-stranded blocking oligonucleotides to at least a subset of non-target polynucleotide sequences before, or during, the same step wherein the analyte target sequence is annealed to a single-stranded probe oligonucleotide A₀ to create a first intermediate product which is at least partially double-stranded and in which the 3′ end of A₀ forms a double-stranded complex with the analyte target sequence.

In some embodiments, the blocking oligonucleotides are made to be resistant to the pyrophosphorolysing reaction via mismatches at their 3′ ends. In another embodiment, the blocking oligonucleotides are made to be resistant via the presence of a 3′-blocking group. In another embodiment the blocking oligonucleotides are made to be resistant via the presence of spacers or other internal modifications. In a further embodiment the blocking oligonucleotides include both a melting temperature increasing modification or modified nucleotide base and are rendered resistant to pyrophosphorolysis.

References herein to ‘phosphatase enzymes’ refer to any enzymes, or functional fragments thereof, with the ability to remove by hydrolysis the nucleoside triphosphates produced by the methods of the current invention. This includes any enzymes, or functional fragments thereof, with the ability to cleave a phosphoric acid monoester into a phosphate ion and an alcohol.

References herein to ‘pyrophosphatase enzymes’ refer to any enzymes, or functional fragments thereof, with the ability to catalyse the conversion of one ion of pyrophosphate to two phosphate ions.

This also includes inorganic pyrophosphatases and inorganic diphosphatases. A non-limiting example is thermostable inorganic pyrophosphate (TIPP).

In some embodiments there is provided a modified version of any previously described embodiment wherein the use of a pyrophosphatase is optional.

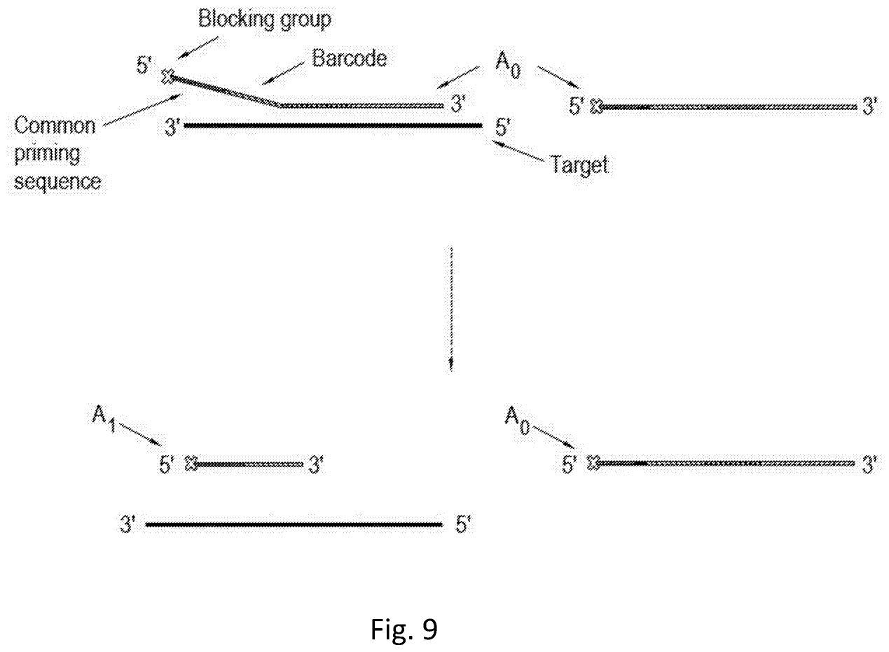

Some embodiments, of the method of the invention can be seen in FIGS. 9 to 12.

In FIG. 9, a single-stranded probe oligonucleotide A₀ anneals to a target polynucleotide sequence to create a first intermediate product which is at least partially double-stranded and in which the 3′ end of A₀ forms a double-stranded complex with the target polynucleotide sequence.

In this simplified embodiment of the invention there are two molecules of A₀ present and one target polynucleotide sequence, in order to illustrate how A₀ that has not annealed to a target does not take part in further steps of the method. In this illustrative example, the 3′ end of A₀ anneals to the target polynucleotide sequence whilst the 5′ end of A₀ does not. The 5′ end of A₀ comprises a 5′ chemical blocking group, a common priming sequence and a barcode region.

The partially double-stranded first intermediate product undergoes pyrophosphorolysis in the presence of a pyrophosphorolysing enzyme in the 3′-5′ direction from the 3′ end of A₀ to create a partially digested strand A₁, the analyte and the undigested A₀ molecule which did not anneal to a target.

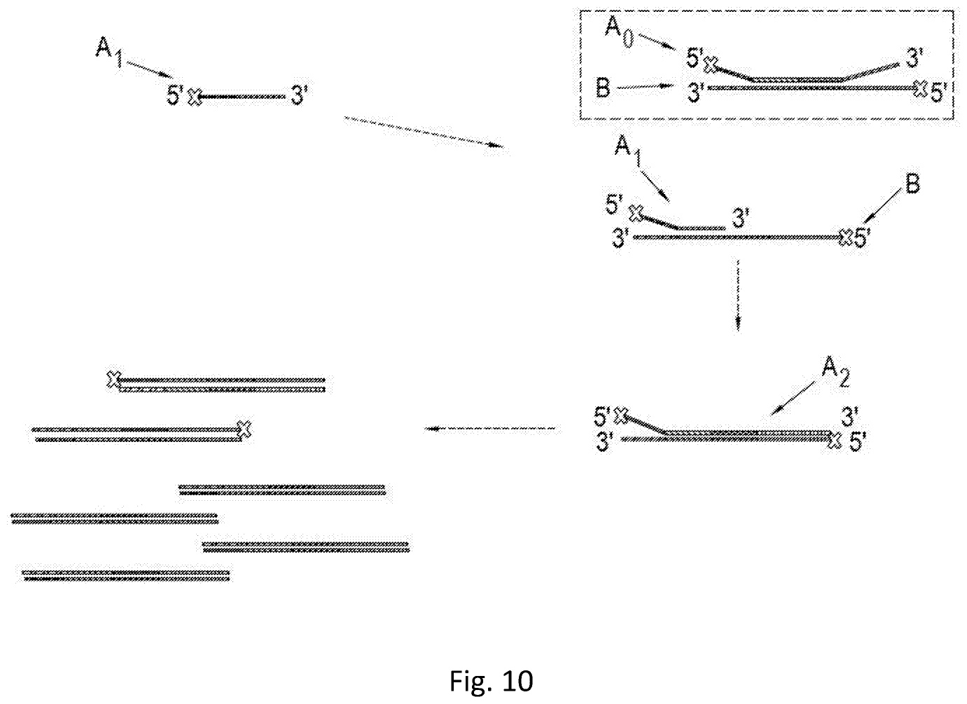

In FIG. 10, A₁ is annealed to a single-stranded trigger oligonucleotide B and the A₁ strand is extended in the 5′-3′ direction against B to create an oligonucleotide A₂. In this illustrative example, trigger oligonucleotide B has a 5′ chemical block. Any undigested A₀ anneals to the trigger oligonucleotide B, however it is unable to be extended in the 5′-3′ direction against B to generate sequences that are the targets for later parts of the method. In this example, A₂ is primed with at least one single-stranded primer oligonucleotide and multiple copies of A₂, or a region of A₂ are created.

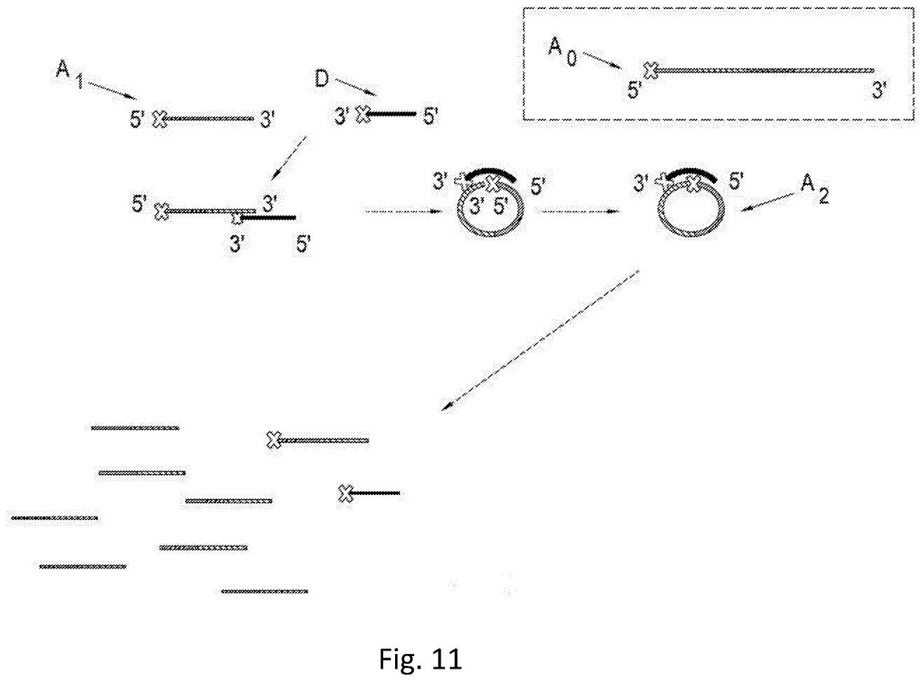

In FIG. 11, A₁ is annealed to a splint oligonucleotide D, and then circularised by ligation of its 3′ and 5′ ends. The now circularised A₂ is primed with at least one single-stranded primer oligonucleotide and multiple copies of A₂, or a region of A₂ are created. In this illustrative example, the splint oligonucleotide D is unable to extend against A₁ by virtue of either a 3′-modification (chemical in this illustration) or through a nucleotide mismatch between the 3′ end of D and the corresponding region of A₂.

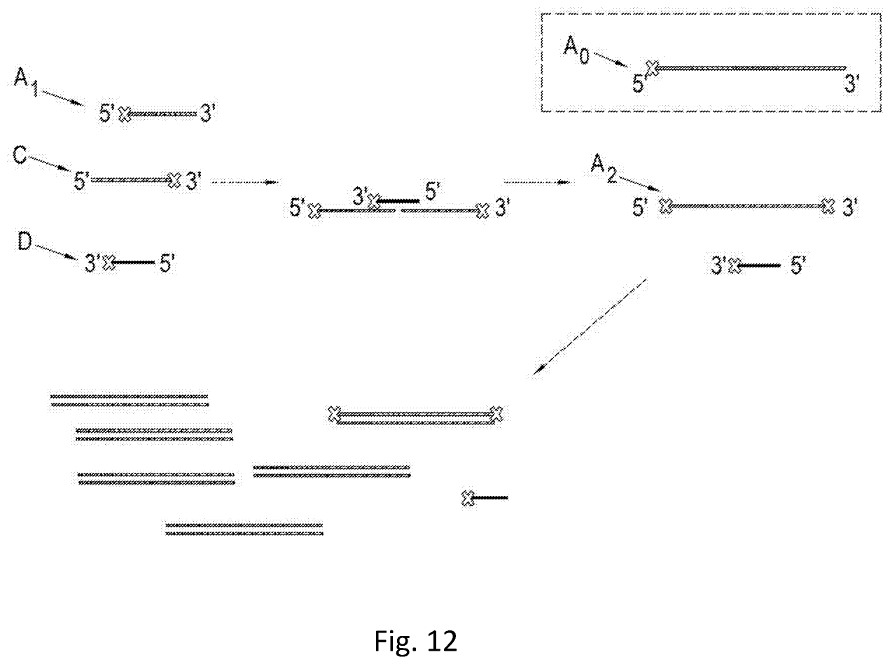

In FIG. 12, the 3′ region of a splint oligonucleotide D anneals to the 3′ region of A₁ whilst the 5′ region of the splint oligonucleotide D anneals to the 5′ region of a ligation probe C. Thus, a second intermediate product A₂ is formed comprised of A₁, C and optionally an intermediate region formed by extension of A₁ in the 5′-3′ direction to meet the 5′ end of C. In this illustrative example, the ligation probe C has a 3′ chemical blocking group so that a 3′-5′ exonuclease can be used to digest any non-ligated A₁.

A₂ is primed with at least one single-stranded primer oligonucleotide and multiple copies of A₂, or a region of A₂ are created.

In some embodiments of the invention there is provided a kit comprising:
(a) a single-stranded probe oligonucleotide A₀, capable of forming a first intermediate product with a target polynucleotide sequence, said intermediate product being at least partially double-stranded;
(b) a capture oligonucleotide B₀ comprising a region complementary to a region adjacent to the target nucleic acid sequence and a capture moiety;
(c) a solid support;
(d) a ligase;
(e) a pyrophosphorolyising enzyme capable of digesting the first intermediate product in the 3′-5′ direction from the end of A₀ to create a partially digested strand A₁; and
(f) suitable buffers.

In some embodiments the kit further comprises a source of pyrophosphate ion.

Suitable source(s) of pyrophosphate ion are as described previously.

In some embodiments, the kit further comprises at least one single-stranded primer oligonucleotide that is substantially complementary to a portion of A₀.

In some embodiments, the kit further comprises an amplification enzyme.

In some embodiments, the capture oligonucleotide is complementary to a region that is within 10000, 5000, 2500, 1000, 500, 250, 100, 90, 80, 70, 60, 50, 40, 30, 20, 10 or 5 nucleotides of the target nucleic acid sequence.

The capture oligonucleotide of the kit may be as previously described.

In some embodiments, the solid support may be as previously described.

In some embodiments, the kit may further comprise reagents suitable for cleavage of A₀, B₀ and/or C₀ as previously described.

In some embodiments, the kit further comprises suitable positive and negative controls.

In some embodiments, the kit may further comprise one or more control probes (E₀) which are as previously described.

In some embodiments, the kit may further comprise one or more blocking oligonucleotides which are as previously described.

In some embodiments, the kit may further comprise one or more control probes (E.) and one or more blocking oligonucleotides which are as previously described.

In some embodiments, the 5′ end of A₀ may be rendered resistant to 5′-3′ exonuclease digestion and the kit may further comprise a 5′-3′ exonuclease.

In some embodiments a kit may further comprise a ligation probe oligonucleotide C.

In some embodiments a kit may further comprise a splint oligonucleotide D.

In some embodiments, a kit may comprise both C and D.

The ligation probe C may comprise a 3′ or internal modification protecting it from 3′-5′ exonuclease digestion.

D may comprise an oligonucleotide region complementary to the 3′ end of A₁ and a region complementary to either the 5′ end of oligonucleotide C or the 5′ end of A₁.

In some embodiments, D may be unable to undergo extension against A₁ by virtue of either a 3′ modification or through a mismatch between the 3′ end of D and the corresponding region of A₁ or C.

In some embodiments, the kit may further comprise dNTPs, a polymerase and suitable buffers for the amplification of a target polynucleotide sequence.

In some embodiments, the kit may further comprise a dUTP incorporating high fidelity polymerase, dUTPs and uracil-DNA N-glycosylase (UDG).

High-fidelity DNA polymerases have several safeguards to protect against both making and propagating mistakes while copying DNA. Such enzymes have a significant binding preference for the correct versus the incorrect nucleoside triphosphate during polymerization. If an incorrect nucleotide does bind in the polymerase active site, incorporation is slowed due to the sub-optimal architecture of the active site complex. This lag time increases the opportunity for the incorrect nucleotide to dissociate before polymerase progression, thereby allowing the process to start again, with a correct nucleoside triphosphate. If an incorrect nucleotide is inserted, proofreading DNA polymerases have an extra line of defense. The perturbation caused by the mispaired bases is detected, and the polymerase moves the 3′ end of the growing DNA chain into a proofreading 3′→5′ exonuclease domain. There, the incorrect nucleotide is removed by the 3′→5′ exonuclease activity, whereupon the chain is moved back into the polymerase domain, where polymerization can continue.

In some embodiments, the kit may further comprise a phosphatase or a phosphohydrolase.

In some embodiments, the kit may further comprise a pyrophosphatase. The pyrophosphatase may be hot start.

In some embodiments, the kit may further comprise a proteinase.

In some embodiments, the kit may further comprise one or more oligonucleotide binding dyes or molecular probes.

In some embodiments, the kit may further comprise multiple A₀, each selective for a different target sequence and each including an identification region.

In some embodiments, the kit further comprises:
two or more Ligation Chain Reaction (LCR) probe oligonucleotides that are complementary to adjacent sequences on A₁ wherein when the probes are successfully annealed the 5′ phosphate of one LCR probe is directly adjacent to the 3′OH of the other LCR probe; and
one or more ligases;

In some embodiments, the kit further comprises:
A ligation probe oligonucleotide C;
A splint oligonucleotide D;
wherein C has a 5′ phosphate, the 3′ end of a splint oligonucleotide D is complementary to the 5′ end of C and the 5′ end of D is complementary to the 3′ end of A₁ such that A₁ and C are capable of being ligated to form A₂.

In some embodiments, the kit further comprises:
A hairpin oligonucleotide 1 (HO1) comprising a fluorophore-quencher pair, wherein HO1 is complementary to A₂ and when annealed to A₂ the hairpin structure of HO1 opens and the fluorophore-quencher pair separate; and
A hairpin oligonucleotide 2 (HO2) comprising a fluorophore-quencher pair, wherein HO2 is complementary to the open HO1 and when annealed to HO1 the hairpin structure of HO2 opens and the fluorophore-quencher pair separate.

In some embodiments, the kit further comprises a plurality of HO1 and HO2.

In some embodiments, the kit further comprises the kit further comprises:
an oligonucleotide A comprising a substrate arm, a partial catalytic core and a sensor arm;
an oligonucleotide B comprising a substrate arm, a partial catalytic core and a sensor arm; and
a substrate comprising a fluorophore quencher pair;
wherein the sensor arms of oligonucleotides A and B are complementary to flanking regions of A₂ such that in the presence of A₂, oligonucleotides A and B are combined to form a catalytically, multicomponent nucleic acid enzyme (MNAzyme).

In some embodiments, the kit further comprises a partially double-stranded nucleic acid construct wherein:
one strand comprises at least one RNA base, at least one fluorophore and wherein a region of this strand is complementary to a region of A₂ and wherein this strand may be referred to as the ‘substrate’ strand; and
the other stand comprises at least one quencher and wherein a region of this strand is complementary to a region of A₂ adjacent to that which the substrate strand is complementary to, such that in the presence of A₂ the partially stranded nucleic acid construct becomes substantially more double-stranded.

In some embodiments, the kit further comprises an enzyme for removal of the at least one RNA base.

In some embodiments of the kit, the enzyme is Uracil-DNA Glycosylase (UDG) and the RNA base is uracil.

In some embodiments, the kit further comprises:
an oligonucleotide complementary to a region of A₂ including the site of ligation, comprising one or multiple fluorophores arranged such that their fluorescence is quenched either by their proximity to each other or to one or more fluorescence quenchers;
a double strand specific DNA digestion enzyme;
wherein, in the presence of A₂, the labelled oligonucleotide is digested such that the fluorophores are separated from each other or from their corresponding quenchers, and a fluorescent signal, and hence the presence of A₂, is detectable.

In some embodiments of the kit, the double strand specific DNA digestion enzyme is an exonuclease.

In some embodiments of the kit, the double strand specific DNA digestion enzyme is a polymerase with proofreading activity.

In some embodiments of the kit, the fluorophore is selected from dyes of the fluorescein family, the carboxyrhodamine family, the cyanine family, the rhodamine family, polyhalofluorescein-family dyes, hexachlorofluorescein-family dyes, coumarin-family dyes, oxazine-family dyes, thiazine-family dyes, squaraine-family dyes, and chelated lanthanide-family dyes.

In some embodiments of the kit, the quencher is selected from those available under the trade designations Black Hole™, Eclipse™. Dark, Qx1J, and Iowa Black™.

In some embodiments, the kit further comprises a phosphatase or a phosphohydrolase.

In some embodiments, the kit further comprises a pyrophosphatase.

In some embodiments, the kit further comprises an enzyme for the transcription of RNA into DNA.

In some embodiments of the kit, the enzyme is a reverse transcriptase.

In some embodiments of the kit, one or more enzymes are hot start.

In some embodiments of the kit, one or more enzymes are thermostable.

In some embodiments, the kit may further comprise suitable washing and buffer reagents.

In some embodiments, the kit further comprises an epigenetic-sensitive and/or an epigenetic-dependent restriction enzyme, which may be as previously described.

In some embodiments, the kit further comprises a methylation-sensitive and/or methylation-dependent restriction enzyme.

In some embodiments, the kit further comprises one or methyl-CpG binding domain (MBD) proteins.

In some embodiments, the kit further comprises one or more 5-methylcytidine (5-mC) antibodies.

In some embodiments, the kit further comprises one or more of MBD2b protein and/or one or more of the MBD2b/MBD3L1 complex.

In some embodiments, the kit further comprises reagents suitable for methylation-specific multiplex ligation-dependent probe amplification (MS-MLPA).

The kit may further comprise purification devices and reagents for isolating and/or purifying a portion of polynucleotides, following treatment as described herein. Suitable reagents are well known in the art and include gel filtration columns and washing buffers.

The person skilled in the art will appreciate that the kit may further comprise one or more of any reagent, component or construct which has been previously described in embodiments of one or more of the methods of the invention.

In some embodiments of the invention there is provided a device comprising at least a fluid pathway between a first, second, third, fourth, fifth and sixth regions, wherein each region comprises one or more wells.

In some embodiments, a sample is introduced to the first region.

In some embodiments, there is a further region connected to one or more of the first, second, third, fourth, fifth or sixth regions by a fluid pathway. In some embodiments, this further region comprises one or more wells. In some embodiments, the one or more wells comprise binding buffer. In some embodiments, the one or more wells comprises wash buffer. In some embodiments, the one or more wells comprise binding and wash buffer.

In some embodiments, the one or more wells of the first region comprise:
A single-stranded probe oligonucleotide A₀ including a region complementary to a target nucleic acid sequence;
A capture oligonucleotide B₀ comprising a region complementary to a region adjacent to the target nucleic acid sequence and a capture moiety; and
A solid support.
In some embodiments, A₀ may be as previously described.

In some embodiments, B₀ may be as previously described.

In some embodiments, the capture moiety is as previously described. In some embodiments, the one or more wells of the first region comprise the reagents necessary for release of B₀ from the solid support. In some embodiment, the device is arranged such that release agents are introduced to one or more regions from one or more separate chambers.

In some embodiments, the solid support is as previously described.

In some embodiments, the solid support is the surface of the one or more wells.

In some embodiments, the solid support is a bead.

In some embodiments, the solid support is a magnetic bead.

In some embodiments, there are a plurality of solid supports.

In some embodiments, the device further comprises one or more magnets. In some embodiments, the one or more magnets are arranged such that the one or more magnetic beads are capable of being moved from one region of the device to another.

In some embodiments, the one or more wells of the first region comprise multiple single-stranded probe oligonucleotides A₀, each complementary to a different target nucleic acid sequence.

In some embodiments, one or more wells of the first region of the device may further comprise an enzyme for the transcription of DNA from an RNA template.

In some embodiments, the enzyme is a reverse transcriptase.

In some embodiments, the one or more wells of the second region comprise reagents for the pyrophosphorolysis reaction. In some embodiments, the one or more wells of the second region comprise:
A source of ions for driving the pyrophosphorolysis reaction;
A pyrophosphorolysis enzyme; and
Suitable buffers.

In some embodiments, the source of ions is a source of pyrophosphate ions.

In some embodiments, the pyrophosphorolysis enzyme is a polymerase.

In some embodiments, the one or more wells of the second region further comprises an enzyme that catalyses the hydrolysis of ATP to yield AMP and inorganic phosphate. In some embodiments, the enzyme is apyrase.

In some embodiments, the one or more wells of the third region comprise ligase reagents. In some embodiments, the one or more wells comprise:
A ligase; and
One or more splint oligonucleotides D.

In some embodiments, the one or more splint oligonucleotides D have a region complementary to the 3′ end of A₁. In some embodiments, the one or more splint oligonucleotides have a further region complementary to the 5′ end of A₁. In some embodiments, the one or more splint oligonucleotides have a further region complementary to the 5′ end of a ligation probe C and wherein the one or more wells of the third region further comprise a ligation probe C.

The ligation probe C may comprise a 3′ or internal modification protecting it from 3′-5′ exonuclease digestion.

D may be unable to undergo extension against A₁ by virtue of either a 3′ modification or through a mismatch between the 3′ end of D and the corresponding region of A₁ or C.

In some embodiments, the one or more wells of the fourth region comprise an enzyme that stops the pyrophosphorolysis enzyme. In some embodiments, the enzyme is a pyrophosphatase.

In some embodiments, the one or more wells of the fifth region comprise PCR reagents. In some embodiments, the one or more wells of the fifth region comprise:
dNTPs; and
one or more primers.

In some embodiments, the one or more wells of the fifth region further comprise a DNA amplification enzyme. In some embodiments, the enzyme is a polymerase.

In some embodiments, the one or more primers are capable of priming to all probes present in the one or more wells of the first region.

In some embodiments, the one or more wells of the sixth region comprise detection reagents.

In some embodiments, the one or more wells of the sixth region comprise dNTPs and one or more primers. In some embodiments, the one or more wells of the sixth region further comprise a DNA amplification enzyme. In some embodiments, the enzyme is a polymerase.

In some embodiments, the one or more wells of the sixth region comprise one or more oligonucleotide binding dyes or molecular probes.

In some embodiments, the one or more wells of the sixth region comprise:
two or more Ligation Chain Reaction (LCR) probe oligonucleotides that are complementary to adjacent sequences on A₁ wherein when the probes are successfully annealed the 5′ phosphate of one LCR probe is directly adjacent to the 3′OH of the other LCR probe; and
one or more ligases; In some embodiments, the one or more regions of the sixth region comprise:
A ligation probe oligonucleotideC;
A splint oligonucleotide D;
wherein C has a 5′ phosphate, the 3′ end of a splint oligonucleotide D is complementary to the 5′ end of C and the 5′ end of D is complementary to the 3′ end of A₁ such that A₁ and C are capable of being ligated to form an oligonucleotide A₂.

In some embodiments, the one or more wells of the sixth region may further comprise:
A hairpin oligonucleotide 1 (HO1) comprising a fluorophore-quencher pair, wherein HO1 is complementary to A₂ and when annealed to A₂ the hairpin structure of HO1 opens and the fluorophore-quencher pair separate; and
A hairpin oligonucleotide 2 (HO2) comprising a fluorophore-quencher pair, wherein HO2 is complementary to the open HO1 and when annealed to HO1 the hairpin structure of HO2 opens and the fluorophore-quencher pair separate.

In some embodiments, the one or more wells of the sixth region may further comprise a plurality of HO1 and HO2.

In some embodiments, the one or more regions of the sixth region comprise:
an oligonucleotide A comprising a substrate arm, a partial catalytic core and a sensor arm;
an oligonucleotide B comprising a substrate arm, a partial catalytic core and a sensor arm; and
a substrate comprising a fluorophore quencher pair;
wherein the sensor arms of oligonucleotides A and B are complementary to flanking regions of A₂ such that in the presence of A₂, oligonucleotides A and B are combined to form a catalytically, multicomponent nucleic acid enzyme (MNAzyme).

In some embodiments, the one or more regions of the sixth region comprise a partially double-stranded nucleic acid construct wherein:
one strand comprises at least one RNA base, at least one fluorophore and wherein a region of this strand is complementary to a region of A₂ and wherein this strand may be referred to as the ‘substrate’ strand; and
the other stand comprises at least one quencher and wherein a region of this strand is complementary to a region of A₂ adjacent to that which the substrate strand is complementary to, such that in the presence of A₂ the partially stranded nucleic acid construct becomes substantially more double-stranded.

In some embodiments, the one or more regions of the sixth region further comprise an enzyme for the removal of the at least one RNA base. In some embodiments, the enzyme is Uracil-DNA Glycosylase (UDG) and the RNA base is uracil.

In some embodiments, the one or more regions of the sixth region comprise:
an oligonucleotide complementary to a region of A₂ including the site of ligation, comprising one or multiple fluorophores arranged such that their fluorescence is quenched either by their proximity to each other or to one or more fluorescence quenchers;
a double strand specific DNA digestion enzyme;
wherein, in the presence of A₂, the labelled oligonucleotide is digested such that the fluorophores are separated from each other or from their corresponding quenchers, and a fluorescent signal, and hence the presence of A₂, is detectable.

In some embodiments, the double strand specific DNA digestion enzyme is an exonuclease.

In some embodiments, the double-strand specific DNA digestion enzyme is a polymerase with proofreading activity.

In some embodiments, the fluorophore is selected from dyes of the fluorescein family, the carboxyrhodamine family, the cyanine family, the rhodamine family, polyhalofluorescein-family dyes, hexachlorofluorescein-family dyes, coumarin-family dyes, oxazine-family dyes, thiazine-family dyes, squaraine-family dyes, and chelated lanthanide-family dyes.

In some embodiments, the fluorophore of the device may be selected from any of the commercially available dyes.

In some embodiments, the quencher of the device is selected from those available under the trade designations Black Hole™, Eclipse™ Dark, Qx1J, Iowa Black™, ZEN and/or TAO.

In some embodiments, the quencher of the device may be selected from any of the commercially available quencher.

In some embodiments, the one or more regions of the sixth region comprise one or more partially double stranded DNA constructs wherein each construct contains one or more fluorophores and one or more quenchers. In some embodiments, when the construct is partially double-stranded the one or more fluorophores and one or more quenchers are located in close enough proximity to each other such that sufficient quenching of the one or more fluorophores occurs.

In some embodiments, the construct is one strand of DNA with a self-complementary region that is looped back on itself.

In some embodiments, the construct comprises one primer of a primer pair.

In some embodiments, the one or more regions of the sixth region further comprise the other primer of a primer pair.

In some embodiments, a portion of the single stranded section of the construct hybridises to A₂ and is extended against it by a DNA polymerase. In some embodiments, the other primer of the primer pair then hybridises to the extended construct, displaying A₂. This primer is then extended against the construct, displacing the self-complementary region. Thus, the one or more fluorophores and one or more dyes are separated sufficiently for a fluorescent signal to be detected, indicating the presence of A₂.

In such an embodiment the construct may be known as a Sunrise Primer.

In some embodiments, the construct comprises two separate DNA strands.

In some embodiments, a portion of the single stranded section of the construct hybridises to A₂ and is extended against it by a DNA polymerase. In some embodiments, the other primer of the primer pair then hybridises to the extended construct, displaying A₂. This primer is then extended against the construct, in the direction of the double stranded section, displacing the shorter of the DNA strands and thus the one or more fluorophores and one or more dyes are separated sufficiently for a fluorescent signal to be detected, indicating the presence of A₂.

In such an embodiment the construct may be known as a Molecular Zipper.

The person skilled in the art will appreciate that for both the Sunrise Primer and Molecular Zipper it is possible for the one or more fluorophores and the one or more quencher pairs to be located at various positions within each respective construct. The key feature is that each pair is located in sufficient proximity to one another that in the absence of A₂, i.e. when no extension and strand displacement has occurred, no fluorescent signal is emitted.

In some embodiments, one or more regions of the device are combined.

In some embodiments, the second and third regions are combined.

In some embodiments, the second and fourth regions are combined.

In some embodiments, the third and fourth regions are combined.

In some embodiments, the second and sixth regions of the device are combined.

In some embodiments, the fourth and fifth regions are combined.

In some embodiments, the second, third and fifth regions are combined.

In some embodiments, the third, fourth and fifth regions are combined.

In some embodiments, the third, fourth, fifth and sixth regions are combined.

In some embodiments, the second, third, fifth and sixth regions are combined.

In some embodiments, the first region may be fluidically connected to a sample container via a fluidic interface.

In some embodiments, heating and/or cooling elements may be present at one or more regions of the device.

In some embodiments, heating and/or cooling may be applied to one or more regions of the device.

In some embodiments, each region of the device may independently comprise at least 100 or 200 wells.

In some embodiments, each region of the device may independently comprise between about 100 and 300, 400, 500, 600, 700, 800, 900, 1000, 1100, 1200, 1300, 1400, 1500 or more wells. The wells may be of any shape and their locations may be arranged in any format or pattern on a substrate.

In some embodiments, the well-substrate can be constructed from a metal (e.g. gold, platinum, or nickel alloy as non-limiting examples), ceramic, glass, or other enzyme-compatible polymer material, or a composite material. The well-substrate includes a plurality of wells.

In some embodiments, the wells may be formed in a well-substrate as blind-holes or through-holes. The wells may be created within a well-substrate, for example, by laser drilling (e.g. excimer or solid-state laser), ultrasonic embossing, hot embossing lithography, electroforming a nickel mold, injection molding, and injection compression molding.

In some embodiments, individual well volume may range from 0.1 to 1500 nl. In some embodiments, 0.5 to 50 nL. Each well may have a volume of about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or 500 nL.

In some embodiments, well dimensions may have any shape, for example, circular, elliptical, square, rectangular, ovoid, hexagonal, octagonal, conical, and other shapes well known to persons of skill in the art.

In some embodiments, well shapes may have cross-sectional areas that vary along an axis. For example, a square hole may taper from a first size to a second size that is a fraction of the first size.

In some embodiments, well dimensions may be square with diameters and depths being approximately equal.

In some embodiments, walls that define the wells may be non-parallel.

In some embodiments, walls that define the wells may converge to a point. Well dimensions can be derived from the total volume capacity of the well-substrate.

In some embodiments, well depths may range from 25 μm to 1000 μm.

In some embodiments, wells may have a depth of 25, 50, 75, 100, 150, 200, 250, 300, 350, 400, 450, 500, 550, 600, 650, 700, 750, 800, 850, 900, 950 or 1000 μm.

In some embodiments, well diameter may range from about 25 μm to about 500 μm.

In some embodiments, wells may have a width of 25, 50, 75, 100, 125, 150, 175, 200, 225, 250, 275, 300, 325, 350, 375, 400, 425, 450, 475 or 500 μm.

In some embodiments, portions of one or more regions of the device may be modified to encourage or discourage fluid adherence. Surfaces defining the wells may be coated with a hydrophilic material (or modified to be hydrophilic), and thus encourage retention of fluid.

In some embodiments, portions of one or more regions of the device, may be coated with a hydrophobic material (or modified to be hydrophobic) and thus discourage retention of fluid thereon. The person skilled in the art will understand that other surface treatments may be performed such that fluid is preferably held within the wells, but not on upper surfaces so as to encourage draining of excess fluid.

In some embodiments, the wells of the well-substrate may be patterned to have a simple geometric pattern of aligned rows and columns, or patterns arranged diagonally or hexagonally.

In some embodiments, the wells of the well-substrate may be patterned to have complex geometric patterns, such as chaotic patterns or isogeometric design patterns.

In some embodiments, the wells may be geometrically separated from one another and/or feature large depth to width ratios to help prevent cross-contamination of reagents.

In some embodiments, the device may comprise one or more auxiliary regions which are usable to provide process fluids, such as oil or other chemical solutions to one or more of the regions of the device. Such auxiliary regions may be fluidically connected to one or more of the regions of the device via one or more membranes, valves and/or pressure severable substrates (i.e. materials that break when subjected to a pre-determined amount of pressure from fluid within an auxiliary region or adjacent portion of the fluid pathway) such as metal foil or thin film.

In some embodiments, the fluid pathway of the device may include extensive torturous portions. A torturous path between the inlet passage of the fluid pathway and one or more of the regions of the device can be helpful for control and handling of fluid processes. A torturous path can help reduce formation of gas bubbles that can interfere with flowing oil through the fluid pathway.

In some embodiments, the device may further comprises a gas permeable membrane which enables gas to be evacuated from the wells of one or more regions of the device, while not allowing fluid to pass through. The gas permeable membrane may be adhered to the well-substrate of the device by a gas permeable adhesive. In some embodiments, the membrane may be constructed from polydimethylsiloxane (PDMS), and has a thickness ranging from 20-1000 μm. In some embodiments the membrane may have a thickness ranging from 100-200 μm.

In some embodiments, all or portions of the well-substrate may contain conductive metal portions (e.g., gold) to enable heat transfer from the metal to the wells. In some embodiments, the interior surfaces of wells may be coated with a metal to enable heat transfer.

In some embodiments, after appropriate reagents have filled the wells of one or more regions of the device, an isolation oil or thermally conductive liquid may be applied to the device to prevent cross-talk.

In some embodiments, the wells of one or more regions of the device may be shaped to taper from a large diameter to a smaller diameter, similar to a cone. Cone-shaped wells with sloped walls enables the use of a non-contact deposition method for reagents (e.g., ink jet). The conical shape also aids in drying and has been found to prevent bubbles and leaks when a gas permeable membrane is present.

In some embodiments, the wells of one or more regions of the device may be filled by advancing a sample fluid (e.g. via pressure) along the fluid pathway of the device. As the fluid passes over the wells of one or more regions of the device, each well becomes filled with fluid, which is primarily retained within the wells via surface tension. As previously described, portions of the well-substrate of the device may be coated with a hydrophilic/hydrophobic substance as desired to encourage complete and uniform filing of the wells as the sample fluid passes over.

In some embodiments, the wells of one or more regions of the device may be ‘capped’ with oil following filling. This can then aid in reducing evaporation when the well-substrate is subjected to heat cycling. In some embodiments, following oil capping, an aqueous solution can fill one or more regions of the device to improve thermal conductivity.

In some embodiments, the stationary aqueous solution may be pressurised within one or more regions of the device to halt the movement of fluid and any bubbles.

In some embodiments, oil such as mineral oil may be used for the isolation of the wells of one or more regions of the device and to provide thermal conductivity. However, any thermal conductive liquid, such as fluorinated liquids (e.g., 3M FC-40) can be used. References to oil in this disclosure should be understood to include such alternatives as the skilled person in the art will appreciate are applicable.

In some embodiments, the device may further comprise one or more sensor assemblies.

In some embodiments, the one or more sensor assemblies may comprise a charge coupled device (CCD)/complementary metal-oxide-semiconductor (CMOS) detector coupled to a fiber optic face plate (FOFP). A filter may be layered on top of the FOPF, and placed against or adjacent to the well-substrate. In some embodiments, the filter can be layered (bonded) directly on top of the CCD with the FOPF placed on top.

In some embodiments, a hydration fluid, such as distilled water, may be heated within the first region or one of the auxiliary regions such that one or more regions of the device has up to 100% humidity, or at least sufficient humidity to prevent over evaporation during thermal cycling.

In some embodiments, after filing of the device is complete, the well-substrate may be heated by an external device that is in thermal contact with the device to perform thermal cycling for PCR.

In some embodiments, non-contact methods of heating may be employed, such as RFID, Curie point, inductive or microwave heating. These and other non-contact methods of heating will be well known to the person skilled in the art. During thermal cycling, the device may be monitored for chemical reactions via the sensor arrangements previously described.

In some embodiments, reagents that are deposited in one or more of the wells of one or more of the regions of the device are deposited in a pre-determined arrangement.

In some embodiments, one or more regions of the device comprise transparent windows to enable reaction progress/conditions to be monitored.

In some embodiments, one or more regions the device are illuminated with a light source and any fluorescence emitted from the one or more regions is detected by one or more sensors. The person skilled in the art will understand there exists numerous sensor/light source combinations which may be used.

In some embodiments there is provided a method comprising:
providing a sample fluid to a fluid pathway of a device wherein the device comprises at least a fluid pathway between a first region, a second region and a third region, wherein the first, second and third regions independently comprise one or more wells;
filling the second region with the fluid from the first region such that one or more wells of the second region is coated with the fluid;
evacuating the fluid from the second region such that one or more wells remain wetted with at least some of the fluid;
filling the third region with the fluid evacuated from the second region such that one or more wells of the third region is coated with this fluid; and
evacuating the fluid from the third chamber such that the one or more wells remains wetted with at least some of this fluid.

In some embodiments of the method, the fluid pathway may be valve less.

In some embodiments of the method, the evacuated second region may be filled with a hydrophobic substance.

In some embodiments of the method, the evacuated third region may be filled with a hydrophobic substance.

In some embodiments of the method, the hydrophobic substance may be supplied from an oil chamber that is in fluid communication with the second and third regions.

In some embodiments of the method, the sample fluid may be routed along the fluid pathway in a serpentine manner.

In some embodiments, the method may further comprise applying heating and cooling cycles to the one or more of the first, second or third regions.

In some embodiments there is provided a method of detecting a target polynucleotide sequence in a given nucleic acid analyte, characterised in that the method is carried out on a device as previously described, the method comprising the steps of:
a. introducing a sample comprising one or more nucleic acid analytes to a first region of a device, said region comprising one or more wells, wherein each well may comprise a reaction mixture, said reaction mixture comprising:
   i. A single-stranded probe oligonucleotide A₀ including a region complementary to the target nucleic acid sequence;
   ii. A capture oligonucleotide B₀ comprising a region complementary to a region adjacent to the target nucleic acid sequence and a capture moiety;
   iii. A solid support;
b. filing a second region of the device with the reaction mixture resulting from (a), wherein the second region comprises one or more wells, wherein each well may comprise a further reaction mixture comprising:
   i. A pyrophosphorolysing enzyme;
   ii. A source of pyrophosphate ions; and
   iii. A ligase;
c. filing a third region of the device with the reaction mixture resulting from (b), wherein the third region comprises one or more wells, wherein each well may comprise a further reaction mixture comprising:
   i. reagents suitable for the detection of the presence of A₂; or
   ii. reagents suitable for the detection of the presence of an oligonucleotide comprising A₂.
wherein the device further comprises detection apparatus capable of detecting the presence or absence of A₂ or an oligonucleotide comprising A₂.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

As used herein, “magnetic microparticles” are magnetically responsive microparticles which are attracted by a magnetic field. The magnetic microparticles used in the methods of the present invention comprise a magnetic metal oxide core, which is generally surrounded by a polymer coat which creates a surface that can bind to DNA, RNA, or PNA. The magnetic metal oxide core is preferably iron oxide, wherein iron is a mixture of Fe²⁺ and Fe³⁺. The preferred Fe²⁺/Fe³⁺ ratio is preferably 2/1, but can vary from about 0.5/1 to about 4/1.

There person skilled in the art will appreciate that when the term ‘infer’ is used, for example, ‘infer the presence of absence of a particular sequence’ it refers to determining the presence or absence of a particular feature based on presence of absence of A₂, or copies of A₂ or a region of A₂ or copies of a region A₂.

### Example 1—Simplified Protocols

For the purpose of this, and following sections, embodiments of the invention are exemplified and referred to as Protocol 1-5 respectively.

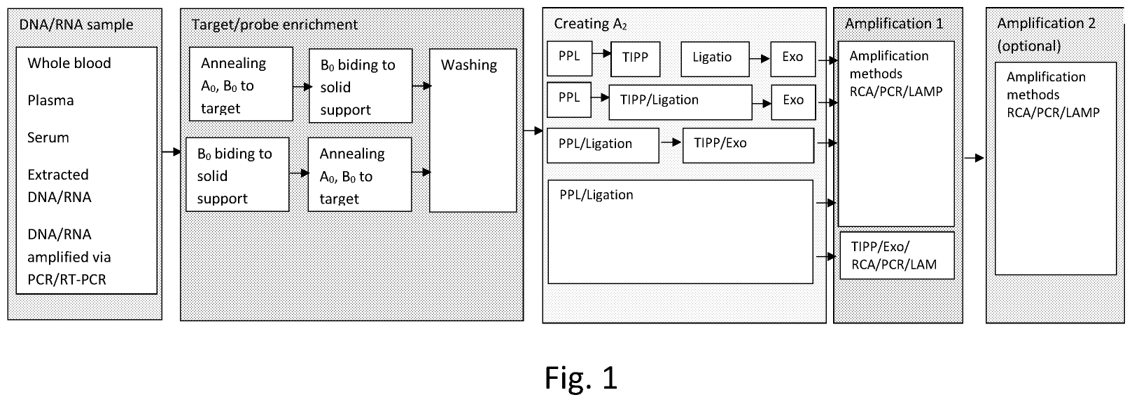

FIG. 1 provides an overview of the different Protocols.

The table below show overviews of the time it may take to perform each Protocol:

Time [min]

| | | | | |
|---|---|---|---|---|
| Step/Protocol | 1 | 2 | 3 | 4 |

| | |
|---|---|
| Target enrichment | 60-90 |

| | | | | |
|---|---|---|---|---|
| PPL | 15 | 15 | 15 | 15 |
| Ligation | 20 | 20 | | |
| TIPP (optional) | 10 | | 10 | 60-100 |
| EXO (optional) | 10 | 10 | | |

| | |
|---|---|
| Amplification 1 | 60-100 |

| | |
|---|---|
| Amplification 2 | 60-100 |

| | | | | |
|---|---|---|---|---|
| (optional) * | | | | |
| Total time | 235-345 | 225-325 | 205-315 | 195-305 |

[* the second amplification is dependent on how many target molecules are assumed to be present in the sample.]

The tables below show example overviews of the enzymes which may be used in each Protocol:

| | Enzyme | | | |
|---|---|---|---|---|
| Step/Protocol | 1 | 2 | 3 | 4 |
| Target | None or cleavage enzyme | | | |
| enrichment | | | | |
| PPL | PPlase | PPlase | PPlase, ligase | PPlase, ligase |
| Ligation | Ligase | Ligase, | | |
| TIPP (optional) | pyrophosphatase | pyrophosphatase | Pyrophosphatase, | Pyrophosphatase, |
| EXO (optional) | 3′→5′ and/or | 3′→5′ and/or | 3′→5′ and/or | 3′→5′ and/or |
| | 5′→3′ exo | 5′→3′ exo | 5′→3′ exo | 5′→ 3′ exo, DNA |
| | | | | polymerase |
| Amplification 1 | DNA polymerase | | | |
| Amplification 2 | DNA polymerase | | | |
| (optional)* | | | | |
| Number of | 7 | 7 | 7 | 7 |
| enzymes | | | | |

[*the second amplification is dependent on how many target molecules are assumed to be present in the sample.]

In one embodiment, TIPP is absent from any one of the protocols, methods, kits and or devices of the invention.

In one embodiment, a 5′-3′ exonuclease is absent from any one of the protocols, methods, kits and or devices of the invention.

In one embodiment, the presence of UDG is optional.

| | Enzyme | | | |
|---|---|---|---|---|
| Step/Protocol | 1 | 2 | 3 | 4 |
| Target | None/restriction endonulease | | | |
| enrichment | | | | |
| PPL | Mako/Klenow/ | Mako/Klenow/ | Mako/Klenow/ | Mako/Klenow/ |
| | Bst L.F/Bst 2.0 | Bst L.F/Bst 2.0 | Bst L.F/Bst 2.0 | Bst L.F/Bst 2.0 |
| | WS/Bsu L.F. | WS/Bsu L.F. | WS/Bsu L.F./ | WS/Bsu L.F./ |
| | | | E. coli | E. coli |
| | | | Ligase/T3 | Ligase/T3 |
| | | | Ligase/T4 | Ligase/T4 |
| | | | Ligase/HiFi | Ligase/HiFi |
| Ligation | E. coli | E. coli | Ligase/9oN | Ligase/9oN |
| | Ligase/T3 | Ligase/T3 | Ligase | Ligase |
| | Ligase/T4 | Ligase/T4 | | |
| | Ligase/HiFi | Ligase/HiFi | | |
| | Ligase/9oN | Ligase/9oN | | |
| | Ligase | Ligase/TIPP/ | | |
| | | YPP | | |
| TIPP | TIPP/YPP | | TIPP/YPP/T5 | TIPP/YPP/T5 |
| (optional) | | | Exo/ExoIII/ | Exo/ExoIII/ |
| | | | ExoT/ExoI | ExoT/ExoI/Bst |
| EXO | T5 | T5 | | L.F./Bst 2.0 |
| (optional) | Exo/ExoIII/ | Exo/ExoIII/ | | WS/phi29/Klenow/ |
| | ExoT/ExoI | ExoT/ExoI | | AmpliTaq/ |
| | | | | TaqPolymerase/ |

| | | |
|---|---|---|
| Amplification | Bst L.F./Bst 2.0 WS/phi29/Klenow/AmpliTaq/ | Q5/PhusionFlex/ |
| 1 | TaqPolymerase/Q5/PhusionFlex/HemoKlenTaq | HemoKlenTaq |

| | |
|---|---|
| Amplification | Bst L.F./Bst 2.0 WS/phi29/Klenow/AmpliTaq/ |
| 2 (optional)* | TaqPolymerase/Q5/PhusionFlex/HemoKlenTaq |

[*the second amplification is dependent on how many target molecules are assumed to be present in the sample.]

As can be seen, the inventors have reduced total number of enzymes needed thus reducing the cost and complexity of the method.

### Example 2—Effect of Pyrophosphorolysis (PPL) Enzyme on PCR Sensitivity

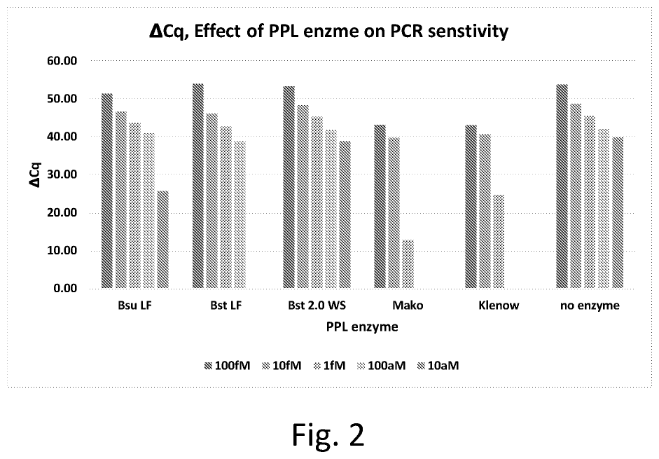

The inventors have investigated the effect of different PPL enzymes on the sensitivity of the PCR reaction. The results of which can be seen in FIG. 2 show that BST 2.0 WS and Bsu LF have no impact on sensitivity. With BST LF, Klenow and Mako the inventors saw a reduction in sensitivity compared to the control (no enzyme).

The experimental protocol used was as follows:

1. PPL step

A PPL mixture (total volume 6 uL) was prepared consisting of:
1×BFF5
PPL enzyme*
100 U/mL E. coli Ligase
0.25 mM inorganic pyrophosphate (PPi)
30 nM splint oligonucleotide
10-100 fM probe oligonucleotide
PPL enzymes used:
Bsu LF 20 U/mL
Bst LF 99 U/mL
Bst 2.0 WS 99 U/mL
Mako 37.5 U/mL
Klenow 10 U/mL
No enzyme
1×BFF5:
TrisHCl pH=7.6 10 mM
KCl 30 mM
MgCl₂ 17.125 mM
Triton-X 0.1%

This mixture was then incubated at 45° C. for 15 minutes.

(SEQ ID NO 1)
Splint oligonucleotide:
5'-ACAGTTTCGGAGTACGAACATCGGTGCGTTTCGCAAG-3'
(SEQ ID NO 2)
Probe oligonucleotide:
5'-
/5Phos/A*T*G*TTCGATGAGCTTTGACAATACTTGACGATTCAT
GCCGTACATGCAGCTATGCAGATATAGGATGTTGCGAAACGCACCG-3'

where * represents a phosphorothioate bond
   2. Detection step
   A. Polymerase Chain Reaction (PCR) mix 1 was prepared corresponding to:
1× Hemo KlenTaq buffer
0.2 uM Primer mix 1
40 rxn/mL Hemo KlenTaq Polymerase
14.67 U/mLTIPP
0.2 uM dNTPs
6 μL of reaction from point 1
Up to 16 uL

### Hemo KlenTaq Buffer:

The Hemo KlenTaq buffer composition is not publically available.

Primer mix 1:

(SEQ ID NO 3)
Fwd: 5'-GGCATGAATCGTCAAGTATT -3'
(SEQ ID NO 4)
Rev: 5'-ATGCAGATATAGGATGTTGC-3'

The resulting mixture was then incubated for:
60 seconds at 95° C.
20× (15 seconds at 95° C.)
15 seconds at 58° C.
15 seconds at 68° C.
5 minutes at 68° C.
5 minutes at 4° C.
   B. A Polymerase Chain Reaction (PCR) mix 2 was prepared corresponding to:
1× Standard Taq buffer
0.2 uM Primer mix 1
0.2 uM labeled probe
26.7 U/mL Taq Polymerase
0.2 uM dNTPs
1.25 uL of reaction from point 2a
Up to 11.25 uL

### Std Taq Buffer Composition

10 mM Tris-HCl
50 mM KCl
1.5 mM MgCl2
(pH 8.3 @ 25° C.)

Primer mix 1:

(SEQ ID NO 3)
Fwd: 5'-GGCATGAATCGTCAAGTATT -3'
(SEQ ID NO 4)
Rev: 5'-ATGCAGATATAGGATGTTGC-3'
(SEQ ID NO 5)
Labeled probe:
5'- /5Cy5/CGCACCGAT/TAO/GTTCGATGAGC/3IAbRQSp/-3'
Where /5Cy5/ - Cy5 dye on 5' end,
/TAO/ - /31AbRQSp/ - double quencher

The resulting mixture was then incubated for:
60 seconds at 95° C.
80× (15 seconds at 95° C.)
15 seconds at 58° C.
15 seconds at 68° C.
5 minutes at 68° C.
5 minutes at 4° C.

A fluorescent reading was performed after every PCR cycle. Cq values were obtained based on the auto-threshold set by a Bio-Rad qPCR machine. The results of this can be seen in FIG. 2.

### Example 3—Sensitivity of Target Enrichment Protocol

The Target Enrichment Protocol allows for the detection of 100 fM mutant molecule.

$$dCq = Cq ( 0 \% AF ) - Cq ( 1 \% AF ) .$$

1. Bead preparation—blocking step
A volume of 1 uL of beads (ThermoFisher Dynabeads MyOne Streptavidin T1 cat. 65601) per sample were put into a 1.5 mL Eppendorftube.
Up to 100 μL of beads can be blocked with 1 mL of blocking solution.
This tube was then placed on top of a magnet.
The storage buffer was removed by aspiration.
To the tube containing the beads 1 mL of blocking buffer was added (1×Phosphate Buffered Saline (PBS) with 1 ug/mL tRNA)
This tube was then rotated at 40 rpm at room temperature (RT) for 30 minutes.
   2. Oligonucleotide annealing
Dilutions of oligonucleotides in 1×AB buffer were prepared (Tris HCl pH=7.5 1 mM, NaCl 50 mL, EDTA 0.2 mM):
Capture oligonucleotide B₀ 100 nM
Probe A₀ 20 nM
Wildtype (WT) oligonucleotide 1 pM-10 nM
Mutant oligonucleotide—10 fM-100 pM

(SEQ ID NO 6)
Capture oligonucleotide:
5'-/5biotin/T
TT
TTTTATCTTTTTGAATTCAGTTTCCTTCATCTAGG-3'
(SEQ ID NO 7)
Probe:
5'-
/5Phos/A*T*G*TTCGATGAGCTTTGACAATACTTGACGATTCA
TGCCGTACATGCAGCTATGCAGATATAGGATGTTGCGAAACGCA
CCGGAGGCCAGCACTTTG-3'
where* represents a phosphorothioate bond
(SEQ ID NO 8)
Wildtype (WT) oligonucleotide:
5'-
AAGGAAACTGAATTCAAAAAGATCAAAGTGCTGGGCTCCGGTGCG
TTCGGCACGGTGTATAAGGTAAGGT-3'
(SEQ ID NO 9)
Mutant oligonucleotide:
5'-
AAGGAAACTGAATTCAAAAAGATCAAAGTGCTGGCCTCCGGTGCG
TTCGGCACGGTGTATAAGGTAAGGT-3'

A 50 uL mixture of the oligonucleotides was prepared per 1 uL of beads The resulting mixture was then incubated at 95° C. for 5 min followed by cooling to room temperature.

3. Attachment of oligonucleotides to the beads

Beads were spun down and placed on top of a magnet. The blocking solution was then removed followed by addition of 10 uL of 2×Binding buffer (TrisHCl pH=7.5 40 mM, NaCl 1M, EDTA 2 mM, Tween20 0.02%) per 1 uL of beads. The beads were mixed with the following ratio:
40 μL of 2×Binding buffer
10 μL of beads
50 μL of annealed oligonucleotides

The beads were then rotated at 40 rpm for 30 min at RT.

### 4. Washing

Beads were then washed with 100 uL 1×Washing buffer (TrisHCl pH=7.5 20 mM, NaCl 0.5 M, EDTA 1 mM, Tween20 0.01%) three times.

5. Wash buffer removal

The wash buffer was then replaced with 100 uL 1×BFF2 buffer with 0.01% Triton-X. 1×BFF2 with 0.01% Triton-X composition:
TrisHCl ph=7.6 10 mM
KCl 30 mM
MgCl₂ 17.125 mM
Triton-X 0.01%
6. Addition of PPL mixture

The 1×BFF2 0.01% Triton-X buffer was then removed followed by the addition of a pyrophosphorolysis (PPL) mixture to the beads (stored at 4*C). PPL mixture composition:
1×BFF2 with 0.1% Triton-X
99 U/mL BST 2.0 WS
100 U/mL E. coli Ligase
0.4 mM inorganic pyrophosphate (PPi)
30 nM splint oligonucleotide
Total volume 6 uL

(SEQ ID NO 1)
Splint oligonucleotide:
5'-ACAGTTTCGGAGTACGAACATCGGTGCGTTTCGCAAG-3'

The resulting mixture was then incubated at 48° C. for 10 minutes.

### 7. Detection

A. A Polymerase Chain Reaction (PCR) mix 1 was prepared corresponding to:
1× Hemo KlenTaq buffer
0.2 uM Primer mix 1
40 rxn/mL Hemo KlenTaq Polymerase
14.67 U/mLTIPP
0.2 uM dNTPs
6 μL of reaction from point 6.
Up to 16 uL

### Hemo KlenTaq Buffer

The Hemo KlenTaq buffer composition is not publically available.

Primer mix 1:

(SEQ ID NO 3)
Fwd:
5'-GGCATGAATCGTCAAGTATT -3'
(SEQ ID NO 4)
Rev:
5'-ATGCAGATATAGGATGTTGC-3'

The mixture was then incubated for:
60 seconds at 95° C.
20× (15 seconds at 95° C.)
15 seconds at 58° C.
15 seconds at 68° C.
5 minutes at 68° C.
5 minutes at 4° C.
   B. A Polymerase Chain Reaction (PCR) mix 2 was prepared corresponding to:
1× Standard Taq buffer
0.2 uM Primer mix 1
0.2 uM labeled probe
26.7 U/mL Taq Polymerase
0.2 uM dNTPs
1.25 uL of reaction from point 7a.
Up to 11.25 uL

### Std Tag Buffer Composition

10 mM Tris-HCl
50 mM KCl
1.5 mM MgCl2
(pH 8.3 @ 25° C.)

Primer mix 1:

(SEQ ID NO 3)
Fwd:
5'-GGCATGAATCGTCAAGTATT -3'
(SEQ ID NO 4)
Rev:
5'-ATGCAGATATAGGATGTTGC-3'
(SEQ ID NO 5)
Labeled probe:
5'- /5Cy5/CGCACCGAT/TAO/GTTCGATGAGC/3IAbRQSp/-3'
Where /5Cy5/ - Cy5 dye on 5' end,
/TAO/ - /31AbRQSp/ - double quencher

The mixture was then incubated for:
60 seconds at 95° C.
80× (15 seconds at 95° C.)
15 seconds at 58° C.
15 seconds at 68° C.
5 minutes at 68° C.
5 minutes at 4° C.

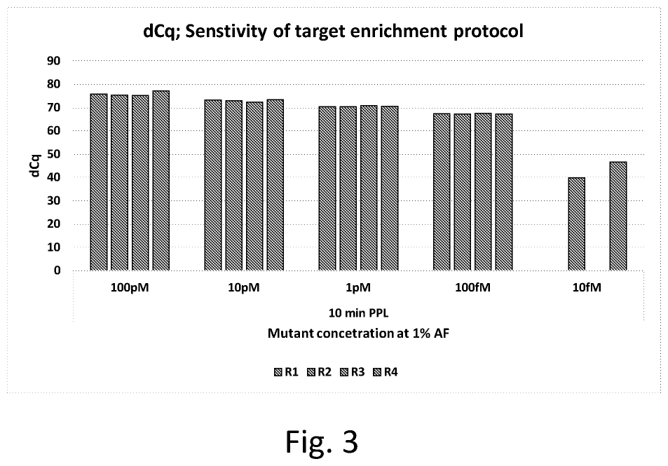

A fluorescent reading was performed after every PCR cycle. Cq values were obtained based on auto-threshold set by Bio-Rad qPCR machine. The results of this can be seen in FIG. 3.

### Example 4: Detection of Target Bound to Beads Vs Target in Solution

### Bead Based Enrichment:

### 1. Bead Preparation—Blocking Step

A volume of 1 uL of beads (ThermoFisher Dynabeads MyOne Streptavidin T1 cat. 65601) per sample were put into a 1.5 mL Eppendorf tube. Up to 100 uL of beads can be blocked with 1 mL of blocking solution.

This tube was then placed on top of a magnet.

The storage buffer was removed by aspiration.

To the tube containing the beads 1 mL blocking buffer was added (1×Phosphate Buffered Saline (PBS) with 1 ug/mL tRNA)

This tube was then rotated at 40 rpm at room temperature (RT) for 30 min

### 2. Oligonucleotide Annealing

Dilutions of oligonucleotides in 1×AB buffer were prepared (Tris HCl pH=7.5 1 mM, NaCl 50 mL, EDTA 0.2 mM)
Capture oligonucleotide B₀ 100 nM
Probe A₀ 20 nM
Wildtype (WT) oligonucleotide 0.019353 nM-20 nM
Mutant oligonucleotide—0.98 pM-125 pM
A 50 uL mixture of the oligonucleotides was prepared per 1 μL of beads

(SEQ ID NO 6)
Capture oligonucleotide:
5'-/5biotin/TT
TTTTTTATCTTTTTGAATTCAGTTTCCTTCATCTAGG-3'
(SEQ ID NO 10)
Probe:
5'-
/5Phos/A*T*G*TTCGATGAGCTTTGACAATACTTGACATGCGCAGATA
TAGGATGTTGCGAAACGCACCGGAGGCCAGCACTTTG-3'
where * represents a phosphorothioate bond
(SEQ ID NO 8)
Wildtype (WT) oligonucleotide:
5'-
AAGGAAACTGAATTCAAAAAGATCAAAGTGCTGGGCTCCGGTGCGTTCG
GCACGGTGTATAAGGTAAGGT
-3'
(SEQ ID NO 9)
Mutant oligonucleotide:
5'-
AAGGAAACTGAATTCAAAAAGATCAAAGTGCTGGCCTCCGGTGCGTTCG
GCACGGTGTATAAGGTAAGGT
-3'

The resulting mixture was then incubated at 95° C. for 5 min followed by cooling to room temperature.

### 3. Attachment of Oligonucleotides to the Beads

a. Beads were spun down and placed on top of a magnet
b. The blocking solution was removed
c. 10 μL of 2×Binding buffer (TrisHCl pH=7.5 40 mM, NaCl 1M, EDTA 2 mM, Tween20 0.02%) was added per every 1 μL of beads
d. The beads were mixed with following ratio:
40 μL of 2×Binding buffer
10 μL of beads
50 μL of annealed oligonucleotides
The beads were then rotated at 40 rpm for 30 min at RT.
   4. Beads were then washed with 100 uL 1×Washing buffer (TrisHCl pH=7.5 20 mM, NaCl 0.5 M, EDTA 1 mM, Tween20 0.1%) three times.
   5. The wash buffer was then replaced with 100 uL 1×BFF5 buffer with 0.01% Triton-X 1×BFF5 with 0.01% Triton-X composition:
TrisHCl pH=7.0 10 mM
KCl 30 mM
MgCl₂ 5 mM
Triton-X 0.01%
   6. The 1×BFF 0.01% Triton-X buffer was then removed followed by the addition of a pyrophosphorolysis (PPL) mixture to the beads (Kept at 4° C.). PPL mixture composition:
1×BFF5 with 0.1% Triton-X
10 U/mL Klenow (exo-)
100 U/mL E. coli Ligase
0.25 mM inorganic pyrophosphate (PPi)
30 nM splint oligonucleotide
Total volume 6 uL

(SEQ ID NO 1)
Splint oligonucleotide:
5'-ACAGTTTCGGAGTACGAACATCGGTGCGTTTCGCAAG-3'

The resulting mixture was then incubated at 45° C. for 15 min

### 7. Detection

a. A Rolling Circle Amplification (RCA) mix 1 was prepared corresponding to:
2.66× Thermopol buffer (53.2 mM Tris-HCl, 26.6 mM (NH₄)₂SO₄, 26.6 mM KCl, 5.32 mM MgSO₄, 0.266% Triton® X-100, pH 8.8)
0.28 uM Primer mix 1
284.4 U/mL BST 2.0 WarmStart
14.67 U/mLTIPP
1.06 mM dNTPs
833.33 U/mL ExoI
166.67 U/mL ExoIII
6 μL of reaction from point 6.
Up 16 uL

Primer mix 1:

(SEQ ID NO 11)
Fwd: 5'-T*C*GCAACATCCTATATC*T*G*C-3'
(SEQ ID NO 12)
Rev: 5'-T*G*AGCTTTGACAATACT*T*G*A-3'
where * represents a phosphorothioate bond

The mixture was then incubated for 10 min at 30° C. and 17.5 min at 50° C.
b. A Rolling Circle Amplification (RCA) mix 2 was then prepared corresponding to:
2.66× Thermopol buffer
0.28 uM Primer mix 2
284.4 U/mL BST 2.0 WarmStart
14.67 U/mLTIPP
1.06 mM dNTPs
833.33 U/mL ExoI
166.67 U/mL ExoIII
30 uM Syto82 dye
1.25 uL of reaction from point 7a.
Up 11.25 uL

Primer mix 2:

(SEQ ID NO 13)
Fwd: 5'-ACATCGGTGCGTTTCGCA*A*C*A-3'
(SEQ ID NO 14)
Rev: 5'-T*G*AGCTTTGACAATACT*T*G*A-3'
where * represents a phosphorothioate bond

The mixture was then incubated for 10 min at 30° C. and 150 min at 50° C.

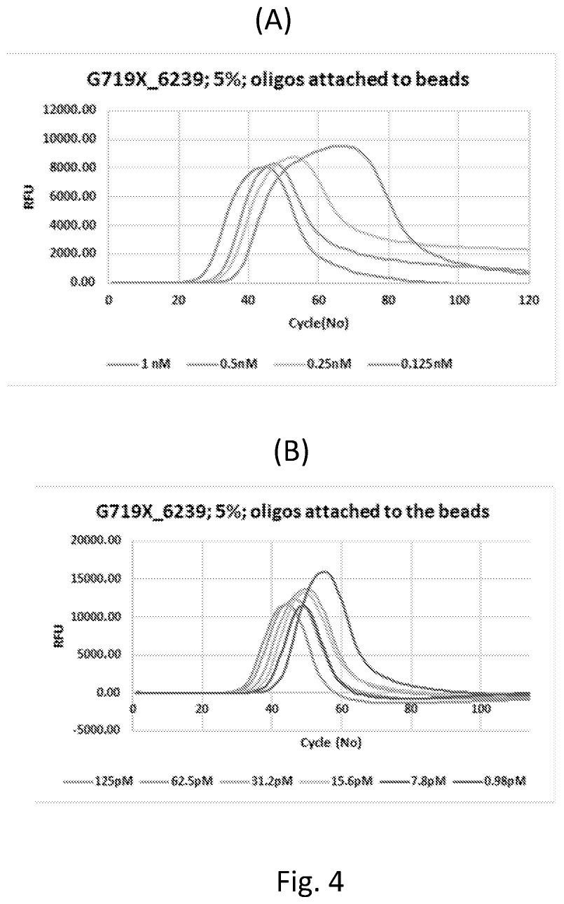

A fluorescent reading was performed after every 1 min. The results of this experiment can be seen FIGS. 4A and 4B.

### Oligonucleotides in Solution

### 1. Oligonucleotide Annealing

A dilution of oligonucleotides in 1×AB buffer (Tris HCl pH=7.5 1 mM, NaCl 50 mL, EDTA 0.2 mM) was prepared corresponding to:
Capture oligonucleotide B₀ 400 nM
Probe A₀ 200 nM
Wildtype (WT) oligonucleotide 0.19353 nM-200 nM
Mutant oligonucleotide—9.8 pM-1250 pM

A 50 uL mixture of the oligonucleotides was prepared.

The resulting mixture was incubated at 95° C. for 5 min and allowed to slowly cool to room temperature.

(SEQ ID NO 6)
Capture oligonucleotide:
5'-/5biotin/T
TTTATCTTTTTGAATTCAGTTTCCTTCATCTAGG-3'
(SEQ ID NO 10)
Probe Ao:
5'-
/5Phos/A*T*G*TTCGATGAGCTTTGACAATACTTGACATGCGCAGATAT
AGGATGTTGCGAAACGCACCGGAGGCCAGCACTTTG-3'
(SEQ ID NO 8)
WT oligonucleotide:
5'-
AAGGAAACTGAATTCAAAAAGATCAAAGTGCTGGGCTCCGGTGCGTTCGG
CACGGTGTATAAGGTAAGGT-3'
(SEQ ID NO 9)
Mutant oligonucleotide:
5'-
AAGGAAACTGAATTCAAAAAGATCAAAGTGCTGGCCTCCGGTGCGTTCGG
CACGGTGTATAAGGTAAGGT-3'

### 2. Pyrophosphorolysis (PPL) and Ligation

A mixture was prepared corresponding to:
1×BFF1
0.0625× A₇ buffer
0.0625× Q5 buffer (NEB)
10 U/mL Klenow (exo−)
100 U/mL E. coli Ligase
1.2 U/mL apyrase
100 U/mL Lambda exo
0.25 mM PPi
30 nM splint oligo
1 μL of oligonucleotide from point 1.
Total volume 10 uL

(SEQ ID NO 1)
Splint oligonucleotide:
5'-ACAGTTTCGGAGTACGAACATCGGTGCGTTTCGCAAG-3'

### 1×BFF1 Composition

Tris Acetate pH=7.0 10 mM
Potassium Acetate 30 mM
Magnesium Acetate 17.125 mM
Triton-X 0.01%

### 1×A7 Composition

Tris Acetate pH=8.0 10 mM
Potassium Acetate 25 mM
Magnesium Acetate 5 mM
Triton-X 0.01%
   Q5 buffer

The Q5 buffer composition is not publically available.

This mixture was then incubated at 45° C. for 15 min.

### 3. Detection—RCA

A mixture corresponding to the following was prepared:
2.66× Thermopol buffer (53.2 mM Tris-HCl, 26.6 mM (NH₄)₂SO₄, 26.6 mM KCl, 5.32 mM MgSO₄, 0.266% Triton® X-100, pH 8.8)
0.28 uM Primer mix
284.4 U/mL BST 2.0 WarmStart
14.67 U/mLTIPP
1.06 mM dNTPs
Syto82 dye 3 uM
1.25 uL of reaction mixture from point 2.
Total volume 11.25 uL

Primer mix:

(SEQ ID NO 15)
Fwd: 5'-TCGCAACATCCTATATCTGC-3'
(SEQ ID NO 16)
Rev: 5'-TGAGCTTTGACAATACTTGA-3'

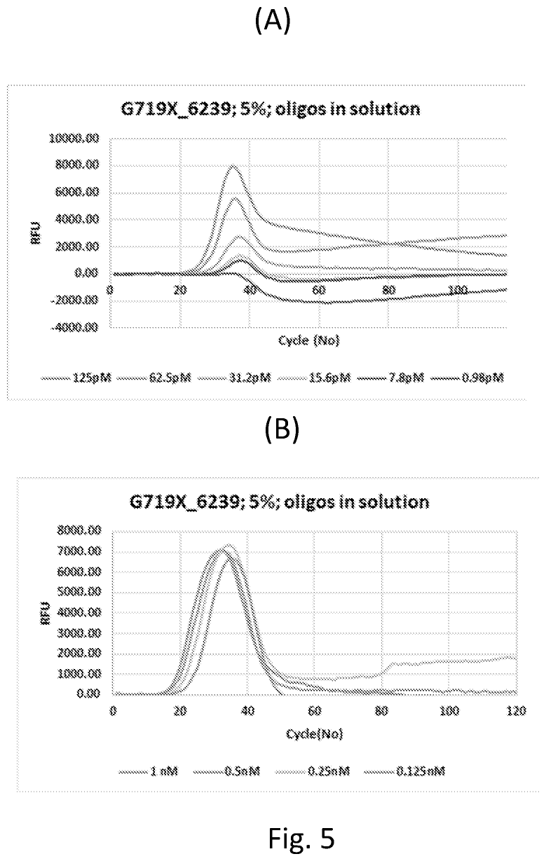

The mixture was then incubated at 50° C. for 90 min. Fluorescent measurements were taken every 1 minute. The results of this experiment can be seen in FIGS. 5A and 5B.

### Example 5: Comparison of Previous Protocol with Target Enrichment

### Target Enrichment

1. Bead preparation

1 ul of beads (ThermoFisher Dynabeads MyOne Streptavidin T1 cat. 65601) per sample were put into a 1.5 mL Eppendorf tube. Up to 100 uL beads can be blocked with 1 mL of blocking solution.

The tube was then placed on a magnet followed by removal of the storage buffer. To the tube was then added 1 mL of blocking buffer (1×PBS with 1 ug/mL tRNA) followed by rotation (40 rpm) at room temperature (RT) for 30 minutes.

2. Oligonucleotide annealing

Dilutions of oligonucleotides in 1×AB buffer (Tris HCl pH=7.5 1 mM, NaCl 50 mL, EDTA 0.2 mM) were prepared.
Capture oligonucleotide B₀ 100 nM
Probe A₀ 20 nM
Double stranded WT oligo 1 pM (mixture of both strands)
Double stranded Mutant oligo—1 fM (mixture of both strands) up to 50 uL

50 μL of each oligonucleotide was prepared per 1 uL of beads, for a total of 50 uL.

The resulting mixtures were then incubated at 95° C. for 5 minutes and then allowed to slowly cool to room temperature.

(SEQ ID NO 6)
Capture oligonucleotide:
5'-/5biotin/T
TTTTTTATCTTTTTGAATTCAGTTTCCTTCATCTAGG-3'
(SEQ ID NO 10)
Probe Ao:
5'-
/5Phos/A*T*G*TTCGATGAGCTTTGACAATACTTGACATGCGCAGATATA
GGATGTTGCGAAACGCACCGG AGGCCAGCACTTTG-3'
where * represents a phosphorothioate bond
(SEQ ID NO 8)
WT oligo:
5'-
AAGGAAACTGAATTCAAAAAGATCAAAGTGCTGGGCTCCGGTGCGTTCGGCA
CGGTGTATAAGGTAAGGT-3'
(SEQ ID NO 17)
WT oligo reverse complementary:
5'-ACCTTACCTTATACACCGTGCCGAACGCACCGGAGCCCAGCACTTTGAT
CTTTTTGAATTCAGTTTCCTT-3'
(SEQ ID NO 9)
Mutant oligo:
5'-
AAGGAAACTGAATTCAAAAAGATCAAAGTGCTGGCCTCCGGTGCGTTCGGCA
CGGTGTATAAGGTAAGGT-3'
(SEQ ID NO 18)
Mutant oligo reverse complementary:
5'-ACCTTACCTTATACACCGTGCCGAACGCACCGGAGGCCAGCACTTTGAT
CTTTTTGAATTCAGTTTCCTT-3'

3. Attachment of oligonucleotides to beads

The beads were spun down for 5 seconds in mini-centrifuge at 2000× g and placed on top of a magnet followed by removal of the blocking solution by aspiration. To the tube was then added 10 μL of 2×Binding buffer (TrisHCl pH=7.5 40 mM, NaCl 1M, EDTA 2 mM, Tween20 0.02%) per every 1 μL of beads.

The ratio of beads/oligos and buffer was as follows:
40 μL of 2×Binding buffer
10 μL of beads
50 μL of annealed oligonucleotides

The tube was then rotated (40 rpm) for 30 minutes at room temperature.

4. Wash step

The beads were then washed with 100 uL 1×Washing buffer (TrisHCl pH=7.5 20 mM, NaCl 0.5 M, EDTA 1 mM, Tween20 0.1%) three times.

5. Exchange beads wash buffer to 100 uL 1×BFF5 buffer with 0.01% Triton-X
1×BFF5 with 0.01% Triton-X composition
TrisHCl pH=7.0 10 mM
KCl 30 mM
MgCl₂ 5 mM
Triton-X 0.01%
   6. Pyrophosphorolysis and ligation

The 1×BFF 0.01% Triton-X buffer was then removed and a PPL mixture (stored at 4° C.) was added to the beads.

The PPL mixture consisted of:
1×BFF5 with 0.1% Triton-X
10 U/mL Klenow (exo-)
100 U/mL E. coli Ligase
0.25 mM PPi
30 nM splint oligo
Total volume 6 uL

(SEQ ID NO 1)
Splint oligonucleotide:
5'- ACAGTTTCGGAGTACGAACATCGGTGCGTTTCGCAAG-3'

The resulting mixture was incubated at 45° C. for 15 minutes.

### 7. Detection

a. A RCA mixture I was prepared corresponding to:
2.66 Thermopol buffer (53.2 mM Tris-HCl, 26.6 mM (NH₄)₂SO₄, 26.6 mM KCl, 5.32 mM MgSO₄, 0.266% Triton® X-100, pH 8.8)
0.28 uM Primer mix
284.4 U/mL BST 2.0 WarmStart
14.67 U/mLTIPP
1.06 mM dNTPs
833.33 U/mL ExoI
166.67 U/mL ExoIII
6 μL of reaction from point 6
Total volume 16 uL

Primer mix:

(SEQ ID NO 11)
Fwd:
5'-T*C*GCAACATCCTATATC*T*G*C-3'
(SEQ ID NO 12)
Rev:
5'-T*G*AGCTTTGACAATACT*T*G*A-3'
where * represents a phosphorothioate bond

The resulting mixture was then incubated 10 minutes at 30° C. and 17.5 minutes at 50° C.

b. A RCA mixture II was prepared corresponding to:
2.66 Thermopol buffer
0.28 uM Primer mix
284.4 U/mL BST 2.0 WarmStart
14.67 U/mLTIPP
1.06 mM dNTPs
833.33 U/mL ExoI
166.67 U/mL ExoIII
30 uM Syto82 dye
1.25 uL of reaction from point 7a
Total volume 11.25 uL

Primer mix

(SEQ ID NO 13)
Fwd:
5'-ACATCGGTGCGTTTCGCA*A*C*A-3'
(SEQ ID NO 14)
Rev:
5' -T*G*AGCTTTGACAATACT*T*G*A-3'
where * represents a phosphorothioate bond

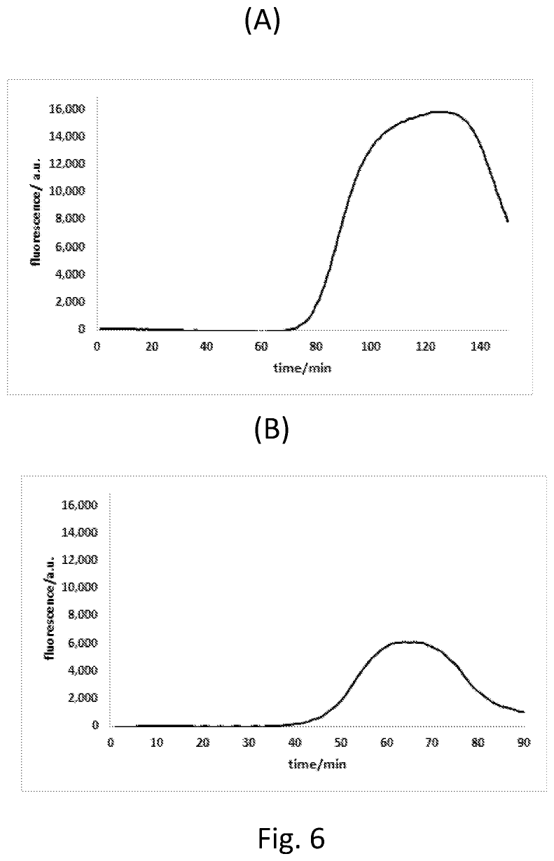

The resulting mixture was then incubated for 10 minutes at 30° C. and 150 minutes at 50° C. Fluorescent readings were taken after every 1 minute. The results can be seen in FIG. 6(A).

### No Target Enrichment

1. Oligonucleotide dilution preparation

Dilution of oligonucleotides were prepared in 0.5×A7 and 0.5×Q5 buffer:
WT oligonucleotide 200 nM
Mutant oligonucleotide 200 pM
Total volume 1.25 uL

(SEQ ID NO 8)
WT oligonucleotide
5-
AAGGAAACTGAATTCAAAAAGATCAAAGTGCTGGGCTCCG
GTGCGTTCGGCACGGTGTATAAGGTAAGGT-3'
(SEQ ID NO 9)
Mutant oligonucleotide:
5'-
AAGGAAACTGAATTCAAAAAGATCAAAGTGCTGGCCTCCG
GTGCGTTCGGCACGGTGTATAAGGTAAGGT-3'

2. Pyrophosphorolysis and ligation

A PPL mixture was prepared consisting of:
1×BFF1
10 U/mL Klenow (exo-)
100 U/mL E. coli Ligase
1.2 U/mL apyrase
200 U/mL Lambda exo
0.25 mM PPi
20 nM Probe A₀
30 nM splint oligo
1.25 uL Oligos from point 1
Total volume 10 uL

(SEQ ID NO 10)
Probe Ao:
5'-
/5Phos/A*T*G*TTCGATGAGCTTTGACAATACTTGACATGCGCAGAT
ATAGGATGTTGCGAAACGCACCGG AGGCCAGCACTTTG-3'
where * Phosphorothioate bonds
(SEQ ID NO 1)
Splint Oligonucleotide:
5'-ACAGTTTCGGAGTACGAACATCGGTGCGTTTCGCAAG-3'

1×BFF1 composition
Tris Acetate pH=7.0 10 mM
Potassium Acetate 30 mM
Magnesium Acetate 17.125 mM
Triton-X 0.01%
   1×A7 composition
Tris Acetate pH=8.0 10 mM
Potassium Acetate 25 mM
Magnesium Acetate 5 mM
Triton-X 0.01%
   Q5 buffer

The Q5 buffer composition is not publically available.

The resulting mixture was incubated at 45° C. for 15 minutes.

### 3. Detection—RCA

A RCA mixture was prepared consisting of:
2.66× Isothermal buffer (53.2 mM Tris-HCl, 26.6 mM (NH₄)₂SO₄, 133 mM KCl, 5.32 mM MgSO₄, 0.266%
Tween20, pH 8.8)
0.28 uM Primer mix
284.4 U/mL BST 2.0 WarmStart
14.67 U/mLTIPP
1.06 mM dNTPs
Syto82 dye 3 uM
1.25 uL of reaction from point 2
Total volume 11.25 uL

Primer mix:

| | (SEQ ID NO 15) |
|---|---|
| Fwd: 5'-TCGCAACATCCTATATCTGC-3' | |
| | (SEQ ID NO 16) |

Rev: 5'-TGAGCTTTGACAATACTTGA-3'

The resulting mixture was incubated at 62° C. for 90 minutes.

Fluorescent readings were taken every 1 minute.

The results of this can be seen in FIG. 6(B).

### Example 6: Pyrophosphorolysis and Ligation Against Target

1. Oligonucleotide dilution preparation

Dilution of oligonucleotides were prepared in 0.5×A7 and 0.5×Q5 buffer:
WT oligonucleotide 200 nM
+/−Mutant oligonucleotide 500 pM
Total volume 1.25 uL

(SEQ ID NO 19)
WT oligonucleotide:
5'-
CTGCTGGGCATCTGCCTCACCTCCACCGTGCAGCTCATCACGCAGCT
CATGCCCTTCGGCTGCCTCCTGGACTATGTCCGG-3'
(SEQ ID NO 20)
Mutant oligonucleotide:
5'-
CTGCTGGGCATCTGCCTCACCTCCACCGTGCAGCTCATCATGCAGCTC
ATGCCCTTCGGCTGCCTCCTGGACTATGTCCGG-3'

2. Pyrophosphorolysis and ligation

A PPL mixture was prepared consisting of:
1×BFF1
10 U/mL Klenow (exo-)
100 U/mL E. coli Ligase
1.2 U/mL apyrase
100 U/mL Lambda exo
0.25 mM PPi
20 nM Probe A₀
1.25 uL Oligos from point 1
Total volume 10 uL

(SEQ ID NO 21)
Probe Ao :
5'-
/5Phos/A*G*C*TGCATCTGAGCTTTGACAATACTTGAGCA
CGGCAGATATAGGATGTTGCGAAGGGCATGAGCTGCATGATG-3'
where * Phosphorothioate bonds

1×BFF1 composition
Tris Acetate pH=7.0 10 mM
Potassium Acetate 30 mM
Magnesium Acetate 17.125 mM
Triton-X 0.01%
   1×A7 composition
Tris Acetate pH=8.0 10 mM
Potassium Acetate 25 mM
Magnesium Acetate 5 mM
Triton-X 0.01%
   Q5 buffer

The Q5 buffer composition is not publicly available.

The resulting mixture was incubated at 45° C. for 15 minutes.

### 3. Detection—RCA

A RCA mixture was prepared consisting of:
2.66× Thermopol buffer (53.2 mM Tris-HCl, 26.6 mM (NH₄)₂SO₄, 26.6 mM KCl, 5.32 mM MgSO₄,
0.266% Triton-X, pH 8.8)
0.28 uM Primer mix
284.4 U/mL BST 2.0 WarmStart
14.67 U/mLTIPP
1.06 mM dNTPs
Syto82 dye 3 uM
1.25 uL of reaction from point 2
Total volume 11.25 uL

Primer mix:

| | |
|---|---|
| | (SEQ ID NO 22) |
| Fwd: | 5'- T*C*GCAACATCCTATATCTGC-3' |
| | (SEQ ID NO 23) |
| Rev: | 5'- ATGTTGCGAAGGGCATATGT-3' |

The resulting mixture was incubated at 50° C. for 70 minutes.

Fluorescent readings were taken every 1 minute. The results can be seen in FIG. 8.

### Example 7: Further Applications of the Methods of the Invention

### Methylation Frequencies of Highly Relevant Methylation Genes (HRMG) in Human Cancers

| Organ | 1 | 2 | 3 | 4 | 5 |
|---|---|---|---|---|---|
| Head and | HOXA9 | NID2 | UCHL1 | DCC | KIF1A |
| neck SCC | (.81, 60%) | (.79, 71%) | (.78, 66%) | (.77, 75%) | (.76, 72%) |
| Esophageal | ZNF582 | NEFH | CDO1 | NMDAR2B | PAX1 |
| SCC | (.95, 86%) | (.93, 86%) | (.91, 84%) | (.91, 78%) | (.89, 100%) |
| Esophageal | SFRP1 | CDO1 | APC | CDH1 | TIMP3 |
| adenocarcinoma | (96%) | (95%) | (92%) | (84%) | (74%) |
| Lung | GHSR | CDO1 | HOXA9 | SHOX2 | TAC1 |
| | (1.0) | (.87, 92%) | (96%) | (94%) | (87%) |
| Stomach | CDO1 | DLEC1 | HOPX | Reprimo | FLNC |
| | (.95, 87%) | (.87, 93%) | (.85, 84%) | (.77, 69%) | (.72, 93%) |
| Large | CDO1 | SFRP1 | GFRA1 | SEPT9 | DCLK1 |
| intestine | (.96, 91%) | (.96, 85%) | (.95, 89%) | (.94, 100%) | (.93, 82%) |
| Biliary | SFRP1 | OPCML | CDO1 | ZSCAN18 | DCLK1 |
| tract | (.95, 84%) | (.93, 89%) | (.91, 85%) | (.77, 65%) | (.75, 58%) |
| Gallbladder | SEPT9 | CDO1 | 14-3-3 sigma | 3-OST-2 | Maspin |
| | (.82, 77%) | (.74, 72%) | (90%) | (72%) | (70%) |
| Pancreas | GHSR | CDO1 | HOPX | NPTX2 | UCHL1 |
| | (1.00) | (.97, 94%) | (.85, 83%) | (100%) | (100%) |
| Breast | GHSR | CDO1 | MAL | 14-3-3 sigma | VGF |
| | (.98, 92%) | (.84, 79%) | (95%) | (91%) | (89%) |
| Uterus | NKX6-1 | SOX9 | SOX1 | ZNF516 | LMX1A |
| (cervical) | (.97, 93%*) | (.96, 92%) | (.95, 88%*) | (.92, 90%) | (.9, 89%*) |
| Uterus | GALR1 | COL14A1 | ZNF177 | ZNF154 | TMEFF2 |
| (endometrial) | (.97, 100%) | (.96, 92%) | (.95, 92%) | (.94, 82%) | (.90, 65%) |
| Bladder | CDO1 | APAF-1 | Twist1 | NID2 | PCDH17 |
| | (.87, 78%) | (100%) | (98%) | (96%) | (92%) |
| Prostate | RASSF1 | MDR1 | APC | GHSR | GSTPI |
| | (.99, 96%) | (.98, 88%) | (.97, 90) | (.97) | (.96, 93%) |

(Area under curve [AUC] of receiver operating characteristic [ROC] curve to differentiate the tumor from the normal counterpart, positive methylation frequency).
Asterisks were assessed by scrape samples.
Area under curve (AUC) could not always endowed in this table due to lack of data.
SCC, squamous cell carcinoma.

### Methylation of Lung Cancer Biomarkers

Lung cancer is a leading cause of cancer-associated mortality for a number of reasons, including its late manifestation of symptoms and the low sensitivity of screening techniques such as chest radiography. DNA fragments shed from tumour cells can provide a convenient and minimally invasive access to the molecular portrait of cancer, with these DNA fragments being found in the cell free DNA (cfDNA) isolated from the blood of cancer patients. Cell-free circulating tumour DNA (ctDNA) in the blood plasma presents a surrogate for the entire cancer genome; ctDNA accounts for as low as 0.05% of total cfDNA or less in many cancer patients, especially in the early stages of the disease. These properties make aberrant methylation of ctDNA a promising cancer biomarker, and recent high throughput investigations have demonstrated the correspondence between the changes in methylation profiles of ctDNA and DNA from paired tumour tissue. A list of methylated markers for lung cancer diagnostics and prognosis are shown below in:

| Gene | Marker | Source | Changes in lung cancer |
|---|---|---|---|
| APC | Adenomatous polyposis coli | Plasma | Significantly changes of |
| | | | methylation levels in lung |
| | | | cancer patients compared |
| | | | with healthy subjects |
| CDH13 | Cadherin 13 | * | * |
| DCLK1 | Doublecortin like kinase 1 | * | * |
| DLEC1 | Deleted in lung and esophageal | * | * |
| | cancer 1 | | |
| LINE-1 | LINE-1 retrotransposable element 1 | * | * |
| P16 | Cyclin-dependent kinase inhibitor 2A | * | * |
| (CDKN2A) | | | |
| RARB2 | Retinoic acid receptor beta 2 | * | * |
| SEPT9 | Septin 9 | * | * |
| SHOX2 | Short stature homeobox 2 | * | * |
| BRMS1 | Breast cancer metastasis suppressor | * | Negative impact on survival |
| | 1 | | |
| DCLK1 | Doublecortin like kinase 1 | * | Negative impact on survival |
| LINE1 | LINE-1 retrotransposable element 1 | * | Dynamic changes of |
| | | | methylation levels in |
| | | | response to antitumor |
| | | | therapy |
| APC | Adenomatous polyposis coli | Serum | Significantly changes of |
| | | | methylation levels in lung |
| | | | cancer patients compared |
| | | | with healthy subjects |
| CDH1 | Cadherin 1 | * | * |
| DAPK | Death-associated protein kinase | * | * |
| DCC | DCC netrin 1 receptor | * | * |
| GSTP1 | Glutathione S-transferase pi 1 | * | * |
| MGMT | O-6-methylguanine-DNA | * | * |
| | methyltransferase | | |
| P16 | Cyclin-dependant kinase inhibitor 2A | * | * |
| (CDKN2A) | | | |
| RASSF1A | Ras association domain family 1 | * | * |
| | isoform A | | |
| TMS1 | PYD and CARD domain containing | * | * |
| CHFR | Checkpoint with forkhead and ring | * | Negative impact on survival |
| | finger domains | | with second-line EGFR-TKIs, |
| | | | compared to chemotherapy |
| SFN | Stratifin | * | Positive impact on survival |
| | | | with platinum-based |
| | | | chemotherapy |

Methylation of TERT and MGMT Promoters and Impact on Brain Cancer The O⁶-methylguanine-DNA methyltransferase (MGMT) gene encodes an evolutionarily conserved and ubiquitously expressed methyltransferase involved in DNA repair. MGMT removes alkyl adducts from the O6-position of guanine, preventing DNA damage and imparting a protective effect on normal cells. However, the endogenous function of MGMT also protects tumour cells from the otherwise lethal effects of chemotherapy with alkylating agents such as temozolomide (TMZ). Silencing or reduced expression of MGMT through methylation of its respective gene promoter has been observed in 50% of grade IV gliomas, compromising DNA repair and as a result increasing chemosensitivity to agents such as TMZ. Therefore, MGMT promoter methylation status has potential as a biomarker of sensitivity to alkylating chemotherapy, ultimately influencing clinical practice. Its capacity as both a predictive and prognostic biomarker has been studied extensively, however, at present there is no consensus on the optimal method of assessment of MGMT gene promoter methylation.

Telomere maintenance protects the integrity of chromosomal ends, enabling replicative immortality, a hallmark of human cancer. The telomere reverse transcriptase (TERT) oncogene encodes the rate-limiting catalytic subunit of the telomerase holoenzyme, which is responsible for telomere maintenance and is normally only expressed in a subset of stem cells. The TERT gene is reactivated in approximately 90% of cancer cells, allowing indefinite proliferation and immortalisation of these cell types. A variety of genetic and epigenetic mechanisms underlying TERT dysregulation have been identified, with hypermethylation of the TERT promoter region representing a unique characteristic of cancer cells. Interestingly, methylation, and not mutation, in the upstream of transcription start site (UTSS) of the TERT gene was found to be strongly associated with increased TERT expression and poor prognosis in paediatric brain tumours. Given the prevalence of TERT promoter hypermethylation in a wide variety of cancer cell types, this epigenetic modification represents a useful prognostic biomarker.

Methylation of Prostate Cancer Genes Etc. Which Genes are/are not Methylated

Prostate cancer is the most frequently diagnosed non-skin malignancy, and a leading cause of cancer-related death in men in Western industrialised countries. There are many DNA methylation changes observed between benign and cancerous prostate tissues, with changes frequently being early and recurrent, suggesting a potential functional role. Several genes and gene families have been reported to be recurrently hypermethylated in prostate cancer by multiple genome-wide studies. This includes, but is not limited to, GSTP1, MGMT, AR, ER, VHL, RB1, APC, DAPK, CD44, AOX1, APC, CDKN2A, HOXD3, PTGS2, RARB, WT1, ZNF154, C20orf103, EFS, HOXC11, LHX9, RUNX3, TBX15, BARHL2, BDNF, CCDC8, CYP27A1, DLX1, EN2, ESR1, FBLN1, FOXE3, GP5, FRSP, HHEX, HOXA3, HOXD4, HOXD8, IRX1, KIT, LBX1, LHX2, NKX2-1, NKX2-2, NKX2-5, PHOXRA, POU3F3, RHCG, SIX6, TBX3, TMEM106, VAX1, and WNT2.

### Methylation of Pancreatic Cancer Markers

Pancreatic ductal adenocarcinoma (PDAC) is one of the most deadly cancer types. This form of cancer is difficult to diagnose as there are currently no early diagnostic tests available, meaning diagnosis usually occurs when the disease is already in an advanced state (>75% of diagnosed cases are stage III/IV diseases). This has led to high mortality rates being recorded. Early diagnosis has proven difficult due to the lack of reliable biomarkers able to capture the early development and/or progression of PDAC. Currently, the only FDA approved biomarker for prognostic surveillance of patients with PDAC is carbohydrate antigen 19-9 (CA19-9 or sialylated Lewis antigen). This antigen displays low sensitivity and specificity for the detection of disease. Its use is therefore discouraged for diagnostic purposes unless used in combination with other circulating biomarkers.

Recent studies have shown that cell-free DNA (cfDNA) methylation analysis represents a promising non-invasive approach for the discovery of biomarkers with diagnostic potential. It may be possible for cfDNA methylation to be utilised for the identification of disease-specific signatures in pre-neoplastic lesions or chronic pancreatitis (CP). As CP often precedes PDAC, dynamic DNA methylation patterns for a given set of genes may underlie the progression of the disease. CUX2, a ductal cell marker, has shown increased signal in PDAC; and REG1A, a ductal and acing cell marker, has shown increasing signal in chronic pancreatitis. Biomarkers ADAMTS1 and BNC1 have been seen to have high methylation frequency in primary PDACs and in pre-neoplastic pancreatic intra-epithelial neoplasia (PINs) (25% and 70% for ADAMTS1 and BNC1, respectively). The combined cfDNA methylation of ADAMTS1 and BNC1 may be utilised for the early diagnosis of pancreatic cancers (i.e. stages I and II). A list of potential biomarkers are shown in the below:

Genes Useful to Identify Circulating Exocrine Pancreatic cfDNA

| | | |
|---|---|---|
| CUX2 | Ductal cell marker | Increased signal in PDAC |
| REG1A | Ductal and acing cell marker | Increasing signal in CP |

Frequency of cfDNA methylation biomarkers (%)

| Gene | healthy | CP | PIN | PDAC I | PDAC II | PDAC III/IV |
|---|---|---|---|---|---|---|
| ADAMTS1 | <3 | — | 25 | 25-88 | 78-90 | 55 |
| BNC1 | 3-7 | 5 | 70 | 63-95 | 56-95 | 100 |
| CCND2 | 17 | 82 | — | — | — | 24 |
| CDKN1C | 60 | 90 | — | — | — | 27 |
| MLH1 | 22 | 78 | — | — | — | 27 |
| PGR (prox) | 14 | 76 | — | — | — | 37 |
| SYK | 57 | 89 | — | — | — | 57 |

### KRAS Detection

The KRAS gene controls cell proliferation, when it is mutated this negative signalling is disrupted and cells are able to continuously proliferate, often developing into cancer. A single amino acid substitution, and in particular a single nucleotide substitution, is responsible for an activating mutation implicated in various cancers: lung adenocarcinoma, mucinous adenoma, ductal carcinoma of the pancreas and colorectal cancer. KRAS mutations have been used as prognostic biomarkers, for example, in lung cancer.

Driver mutations in KRAS are associated with up to 20% of human cancers and there are targeted therapies in development against this mutation and its associated disease(s), a non-limiting list of some such therapies can be seen in the table below:

| | | | | Brand |
|---|---|---|---|---|
| Name | Target | Manufacturer | Status | Name(s) |
| AMG 510 | KRAS^{G12C} | Amgen | Trials | N/A |
| MRTX-849 | KRAS^{G12C} | Mirati | Trials | N/A |
| | | Therapeutics | | |
| ARS-3248 | KRAS^{G12C} | Wellspring | Trials | N/A |
| | | Biosciences, | | |
| | | Inc/Janssen | | |
| | | Biotech, Inc. | | |

The presence of KRAS mutation has been found to reflect a very poor response to the EGFR inhibitors panitumumab (Vectibix) and cetuximab (Erbitux). Activating mutations in the gene that encodes KRAS occurs in 30%-50% of colorectal cancers and studies show that patients whose tumours express this mutated version of the KRAS gene will not respond to panitumumab and cetuximab. The presence of the wild-type KRAS gene is not a guarantee that a patient will respond to these drugs, however, studies have shown that cetuximab has significant efficacy in metastatic colorectal cancer patients with wild-type KRAS tumours. Lung cancer patients who are positive for KRAS mutation (wild-type EGFR) have a response rate estimated at 5% or less for the EGFR antagonists erlotinib or gefitinib compared with a 60% response rate in patients who do not possess a KRAS mutation.

The early detection of the emergence of KRAS mutations (activating or over-expression), a frequent driver of acquired resistance to cetuximab therapy (anti-EGFR therapy) in colorectal cancers, allows the modification of treatment (for example the early initiation of a mitogen-activated protein kinase kinase [MEK] inhibitor) to delay or reverse resistance and thus it is advantageous that the methods of the current invention allow the quick and cheap detection of the KRAS status of patients.

A non-limiting list of mutations is: G12D, G12A, G12C, G13D, G12V, G12S, G12R, A59T/E/G, Q61H, Q61K, Q61R/L, K117N and A146P/T/V.

A further non-limiting list of mutations is shown in the table below:

| | Mutation | | Mutation |
|---|---|---|---|
| Exon | name | COSM Number | sequence |
| 2 | G12A | COSM522 | c.35G > C |
| 2 | G12C | COSM516 | c.34G > T |
| 2 | G12D | COSM521 | c.35G > A |
| 2 | G12F | COSM512 | c.34_35delinsTT |
| 2 | G12R | COSM518 | c.34G > C |
| 2 | G12S | COSM517 | c.34G > A |
| 2 | G12V | COSM520 | c.35G > T |
| 2 | G12V | COSM515 | c.35_36delinsTC |
| 2 | G13A | COSM533 | c.38G > C |
| 2 | G13C | COSM527 | c.37G > T |
| 2 | G13D | COSM532 | c.38G > A |
| 2 | G13R | COSM529 | c.37G > C |
| 2 | G13S | COSM528 | c.37G > A |
| 3 | Q61E | COSM550 | c.181C > G |
| 3 | Q61H | COSM1146992 | c.183A > T |
| 3 | Q61H | COSM554/COSM1135364 | c.183A > C |
| 3 | Q61K | COSM549/COSM1159597 | c.181C > A |
| 3 | Q61L | COSM553 | c.182A > T |
| 3 | Q61R | COSM552 | c.182A > G |

### BRAF Detection

BRAF is a human gene that encodes for a protein called B-Raf which is involved in sending signals inside cells which are involved in directing cell growth. It has been shown to be mutated in some human cancers. B-Raf is a member of the Raf kinase family of growth signal transduction protein kinases and plays a role in regulating the MAP Kinase/ERKs signalling pathway, which affects, amongst other things, cell division.

Certain other inherited BRAF mutations cause birth defects.

More than 30 mutations of the BRAF gene have been identified that are associated with human cancers. In 90% of cases, thymine is substituted with adenine at nucleotide 1799. This leads to valine (V) being substituted for by glutamate (E) at codon 600 (now referred to as V600E) in the activation segment found in human cancers. This mutation has been widely observed in:
Colorectal cancer
Melanoma
Papillary thyroid carcinoma
Non-small cell lung cancer
Ameloblastoma

A non-limiting list of other mutations which have been found are: R4611, 1462S, G463E, G463V, G465A, G465E, G465V, G468A, G468E, N580S, E585K, D593V, F594L, G595R, L596V, T5981, V599D, V599E, V599K, V599R, V600K and A727V.

Drugs that treat cancers driven by BRAF mutations have been developed; vemurafenib and dabrafenib are approved by the FDA for the treatment of late-stage melanoma. The response rate to treatment with vumerafenib was 53% for metastatic melanoma, compared to 7-12% for the former best chemotherapeutic dacarbazine.

### ERBB2/HER2 Detection

Human epidermal growth factor receptor 2 (HER2), also known as CD340 (cluster of differentiation 340), proto-oncogene Neu, Erbb2 (rodent) or ERBB2 (human) is a protein encoded by the ERBB2 gene. Amplification or over-expression of this oncogene plays an important role in the progression of aggressive types of breast cancer. Over-expression of the ERBB2 gene is also known to occur in ovarian, stomach, adenocarcinoma of the lung, and aggressive forms of uterine cancer and 30% of salivary duct carcinomas. Structural alterations have also been identified that cause ligand-independent firing of the receptor in the absence of over-expression.

There are numerous targeted therapies approved and in development against this mutation and its associated disease(s), a non-limiting list of some such therapies can be seen in the table below:

| | | | | Brand |
|---|---|---|---|---|
| Name | Target | Manufacturer | Status | Name(s) |
| Trastuzumab | HER2 | Genentech | Approved | Herceptin |
| | overexpression | | | |
| Pertuzumab | Dimerisation of | Genentech | Approved | Perjeta |
| | HER2 and HER3 | | | |
| | receptors | | | |
| Margetuximab | HER2 | Raven | Trials | N/A |
| | overexpression | biotechnologies/MacroGenics | | |
| NeuVax | Vaccine | Galena Biopharma | Trials | N/A |

HER2 testing is routinely performed in breast cancer patients to assess prognosis, monitor response to treatment and to determine suitability for targeted therapy (trastuzumab etc.). As trastuzumab is expensive and associated with serious side effects (cardiotoxicity) it is important that only HER2+ patients are selected to receive it and thus it is advantageous that the methods of the current invention allow the quick and cheap detection of the HER2 status of patients.

In one embodiment, the presence of absence of the ERRB2 Exon 20 insertion mutations is detected using the methods of the current invention.

A further non-limiting list of ERBB2 mutations is shown in the table below:

| Exon | Mutation name | COSM Number | Mutation sequence |
|---|---|---|---|
| 20 | A775_G776insYVMA | COSM12558 | c.2324_2325ins > TTACGTGATGGC |
| | G778_P780dup | COSM12556 | c.2332_2340dup > GGCTCCCCA |
| | G776delinsVC | COSM1651739 | c.2326_2327ins > TGT |
| | P780_Y781insGSP | COSM21607 | c.2339_2340ins > TGGCTCCCC |
| | A775_G776insYVMA/Y772_A775 dup | COSM20959 | c.2313_2324dup > ATACGTGATGGC |
| 17 | V659E_AA | COSM3724566 | c.1976_1977delinsTT > AA |
| | V659E_AG | COSM6503262 | ? c.1976_1977delinsTT > AG |

### EML4-ALK Detection

EML4-ALK is an abnormal gene fusion of echinoderm microtubule-associated protein-like 4 (EML4) gene and anaplastic lymphoma kinase (ALK) gene. This gene fusion leads to the production of the protein EML4-ALK, which appears to promote and maintain the malignant behaviour of cancer cells. EML4-ALK positive lung cancer is a primary malignant lung tumour whose cells contain this mutation.

There are numerous targeted therapies approved and in development against this mutation and its associated disease(s), a non-limiting list of some such therapies can be seen in the table below:

| Name | Generation | Target | Manufacturer | Status | Brand Name(s) |
|---|---|---|---|---|---|
| Crizotinib | 1 | EML4/ALK | Pfizer | FDA approved 2011 | Xalkori, Crizalk |
| Certinib | 2 | ALK | Novartis | FDA approved 2014 | Zykadia |
| Alectinib | 2 | ALK | Genentec | Japan 2014, FDA approved 2015 | Alecensa |
| Brigatinib | 2 | ALK/EGFR | Takeda | FDA approved 2017 | Alunbrig, Briganix |
| Ensartinib | 2 | ALK | XCovery | Trial(s) | N/A |
| Lorlatinib | 3 | ROS1/ALK | Pfizer | Trial(s) | N/A |

EML4-ALK gene fusions are responsible for approximately 5% of non-small cell lung cancers (NSCLC), with about 9,000 new cases in the US per year and about 45,000 worldwide.

There are numerous variants of EML4-ALK with all variants having the essential coiled-coil domain in the EML4 N-terminal portion and in the kinase domain of ALK exon 20 that are needed for transforming activity. Fusion of exon 13 of EML4 with exon 20 of ALK (variant 1: V1) (detection of which can be seen in FIG. 20), exon 20 of EML4 with exon 20 of ALK (V2), and exon 6 of EML4 with exon 20 of ALK (V3) are some of more common variants. The clinical significance of these different variants has only recently become clearer.

V3 has emerged as a marker suitable for the selection of patients who are likely to have shorter progression-free survival (PFS) after non-tyrosine kinase inhibitor (TKI) treatment such as chemotherapy and radiotherapy. There is further evidence that V3 is associated with shorter PFS of those patients who receive first- and second-generation treatment lines and worse overall survival (OS) compared to V1 and V2 of EML4-ALK.

It has also been found that V3-positive patients develop resistance to first and second treatment lines though the development of resistance mutations and possibly facilitated by incomplete tumour cell suppression due to a higher IC50 of wild-type V3. Detection of the unfavourable V3 could be used to select patients requiring more aggressive surveillance and treatment strategies. It appears that administration of the third generation Lorlatinib to patients with V3 may confer longer PFS over those with V1 and thus it is advantageous that the methods of the current invention allow the quick and cheap detection of which variant a patient may have.

The methods of the current invention further allow the detection of resistance mutations such as, but not limited to: G1202R, G1269A, E1210K, D1203, S1206C, L1196M, F1174C, 11171T, 11171N/S, V1180L, T1151K and C1156Y.

G1202R, for example, is a solvent-front mutation which causes interference with drug binding and confers a high level of resistance to first- and second-generation ALK inhibitors. Thus it is advantageous that the methods of the current invention allow identification of those patients who may possess this mutation and benefit from treatment initiation on a third generation treatment rather than a first or second.

A further non-limiting list of EML4-ALK mutations is shown in the table below:

| | | COSM | Mutation |
|---|---|---|---|
| Exon | Mutation | Number | sequence |
| Rearrangements | EML4-ALK | COSF408 | E13_A20 |
| (always exon 20) | | COSF474 | E6ins33_A20 |
| | | COSF409 | E20_A20 |
| | | COSF411 | E6_A20 |

### EGFR Detection

The identification of the epidermal growth factor receptor (EGFR) as an oncogene led to the development of targeted therapies such as gefitinib, erlotinib, afatinib, brigatinib and icotinib for lung cancer, and cetuximab for colon cancer. However, many people develop resistance to these therapies. Two primary sources of resistance are the T790M mutation and the MET oncogene.

EGFR mutations occur in EGFR exons 18-21 and mutations in exons 18, 19 and 21 and indicate suitability for treatment with EGFR-TKIs (tyrosine kinase inhibitors). Mutations in exon 20 (with the exception of a few mutations) show the tumours are EGFR-TKI resistant and not suitable for treatment with EGFR-TKIs.

The two most common EGFR mutations are short in-frame deletions of exon 19 and a point mutation (CTG to CGG) in exon 21 at nucleotide 2573, which results in substitution of leucine by arginine at codon 858 (L858R) Together, these two mutations account for ˜90% of all EGFR mutations in non-small cell lung cancer (NSCLC). Screening for these mutations in patients with NSCLC can be used to predict which patients will respond to TKIs.

Thus it is advantageous that the methods of the current invention allow identification of those patients who may possess these mutations and benefit from treatment initiation on TKIs. The person skilled in the art will appreciate that the methods of the current invention allow identification of a range of EGFR mutations, a non-exhaustive list of such mutations is: G719X, Ex19Del, S7681, Ex201 ns and L861Q.

A further non-limiting list of mutations is shown in the table below:

| Exon | Mutation name | COSM Number | Mutation sequence |
|---|---|---|---|
| Exon 18 | G719A | 6239 | c.2156G > C |
| | G719S | 6252 | c.2155G > A |
| | G719C | 6253 | c.2155G > T |
| Exon 19 | del_6210 | 6210 | 2240_2251del12 |
| | del_6218 | 6218 | 2239_2247del9 |
| | del_6220 | 6220 | 2238_2255del18 |
| | del_6223 | 6223 | 2235_2249del15 |
| | del_6225 | 6225 | 2236_2250del15 |
| | del_6255 | 6255 | 2239_2256del18 |
| | del_12367 | 12367 | 2237_2254del18 |
| | del_12369 | 12369 | 2240_2254del15 |
| | del_12370 | 12370 | 2240_2257del18 |
| | del_12382 | 12382 | 2239_2248TTAAGAGAAG > C |
| | del_12383 | 12383 | 2239_2251 > C |
| | del_12384 | 12384 | 2237_2255 > T |
| | del_12385 | 12385 | 2235_2255 > AAT |
| | del_12386 | 12386 | 2237_2252 > T |
| | del_12387 | 12387 | 2239_2258 > CA |
| | del_12403 | 12403 | 2239_2256 > CAA |
| | del_12416 | 12416 | 2237_2253 > TTGCT |
| | del_12419 | 12419 | 2238_2252 > GCA |
| | del_12422 | 12422 | 2238_2248 > GC |
| | del_12678 | 12678 | 2237_2251del15 |
| | del_12728 | 12728 | 2236_2253del18 |
| | del_13550 | 13550 | 2235_2248 > AATTC |
| | del_13551 | 13551 | 2235_2252 > AAT |
| | del_13552 | 13552 | 2235_2251 > AATTC |
| | del_13556 | 13556 | 2253_2276del24 |
| | del_18427 | 18427 | 2237_2257 > TCT |
| | del_26038 | 26038 | 2233_2247del15 |
| | del_6256 | 6256 | ? c.2254_2277del24 |
| | del_28517 | 28517 | ? c.2235_2246del12 |
| | del_1190791 | 1190791 | ? c.2234_2248del15 |
| | del_26718 | 26718 | ? c.2250_2264del15 |
| | del_133189 | 133189 | ? c.2236_2256del21 |
| | del_24970 | 24970 | ? c.2239_2262del24 |
| | I740_K745dup | 26443 | c.2217_2234dup |
| | K745_E746insVPVAIK | 26444 | c.2219_2236dup |
| Exon 20 | T790M | 6240 | 2369C > T |
| | S768I | 6241 | 2303G > T |
| | p.A767_V769dup | 12376 | 2307_2308ins9GCCAGCGTG |
| | p.H773dup | 12377 | 2319_2320insCAC |
| | p.D770_N771insG | 12378 | 2310_2311insGGT |
| | p.S768_D770dup | 13428 | 2311_2312ins9GCGTGGACA |
| | p.A767_V769dup | 13558 | 2309_2310AC > CCAGCGTGGAT |
| | A763_Y764insFQEA | 26720 | 2284-5_2290dupTCCAGGAAGCCT |
| | C797S_2389 | 6493937 | 2389T > A |
| | C797S_2390 | 5945664 | 2390G > C |
| Exon 21 | L858R | 6224 | c.2573T > G |
| | L858R | 12429 | 2573_2574TG > GT |
| | L861Q | 6213 | c.2582T > A |

### ROS1

ROS1 is a receptor tyrosine kinase (encoded by the gene ROS1) with structural similarity to the anaplastic lymphoma kinase (ALK) protein; it is encoded by the c-ros oncogene.

A non-limiting list of ROS1 mutations is shown in the table below:

| | Mutation | COSM | Mutation |
|---|---|---|---|
| Exon | name | Number | sequence |
| Rearrangements | CD74-ROS1 | COSF1202 | C6_R32 |
| | | COSF1200 | C6_R34 |
| | | COSF1478 | C6_R35 |
| | SLC34A2-ROS1 | COSF1196 | S4_R32 |
| | | COSF1198 | S4_R34 |
| | | COSF1259 | S13del2046_R32 |
| | | COSF126 | S13del2046_R34 |
| | SDC4-ROS1 | COSF1265 | S2_R32 |
| | | COSF1278 | S4_R32 |
| | | COSF1671 | S2_R34 |
| | | COSF1280 | S4_R34 |
| | EZR-ROS1 | COSF1267 | E10_R34 |
| | GOPC-ROS1 | — | G4_R35 |
| | | COSF1188 | G4_R36 |
| | | COSF1139 | G8_R35 |
| | LRIG3-ROS1 | COSF1269 | L16_R35 |
| | TPM3-ROS1 | COSF1273 | T8_R35 |

### RET Proto-Oncogene

The RET proto-oncogene encodes a receptor tyrosine kinase for members of the glial cell line-derived neurotrophic factor (GDNF) family of extracellular signalling molecules.

A non-limiting list of RET mutations is shown in the table below:

| | | | Mutation |
|---|---|---|---|
| Exon | Mutation name | COSM Number | sequence |
| Rearrangements | KIF5B-RET | COSF1232 | K15-R12 |
| | | COSF1230 | K16-R12 |
| | | COSF1253 | K22-R12 |
| | | COSF1234 | K23-R12 |
| | | COSF1255 | K15-R11 |
| | | (actually | |
| | | 15_11del107) | |
| | | COSF1262 | K24-R11 |
| | | COSF1242 | K24-R8 |
| | | | K24-R7 |
| | CCDC6-RET | COSF1271 | C1-R12 |
| | NCOA4-RET | COSF1341 | N6-R12 |
| | TRIM33-RET | | T14-R12 |

### MET Exon 14

MET exon 14 skipping occurs with an approximately 5% frequency in NSCLC and is seen in both squamous and adenocarcinoma histology.

A non-limiting list of MET mutations is shown in the table below:

| Exon | Mutation name | COSM Number | Mutation sequence |
|---|---|---|---|
| Skipping 14 | MET-MET | COSM29312 | M13_M15 |

### NTRK Proto-Oncogenes

NTRK gene fusions lead to abnormal proteins called TRK fusion proteins, which may cause cancer cells to grow. NTRK gene fusions may be found in some types of cancer, including cancers of the brain, head and neck, thyroid, soft tissue, lung, and colon. Also called neurotrophic tyrosine receptor kinase gene fusion.

A non-limiting list of NTRK mutations is shown in the table below:

| | | | Mutation |
|---|---|---|---|
| Exon | Mutation name | COSM Number | sequence |
| NTRK1 | CD74-NTRK1 | | C7-N10 |
| Rearrangements | MPRIP-NTRK1 | | M22-N12 |
| | TFG-NTRK1 | COSF1323 | T5-N10 |
| | TPM3-NTRK1 | COSF1329 | T8-N10 |
| NTRK3 | ETV6-NTRK3 | COSF571 | E5-N15 |
| Rearrangements | ETV6-NTRK3 | COSF1534 | E4-N14 |
| | ETV6-NTRK3 | COSF823 | E4-N15 |

### Panels

In one embodiment of the invention, there is provided a panel comprising a plurality of probe molecules (A₀) wherein each A₀ is complementary to a target mutation. The mutation may be selected from any mutation previously, or subsequently, described or known. The person skilled in the art will thus appreciate that within the scope of the invention are included panels which may be useful in the detection of one or more mutations to the any of the proto-oncogenes or oncogenes previously, or subsequently, described or known.

In one embodiment, the panel comprises 5-500 individual probe molecules, each complementary to a specific target mutation. In one embodiment, the panel comprises 5-400 individual probe molecules, each complementary to a specific target mutation. In one embodiment, the panel comprises 5-300 individual probe molecules, each complementary to a specific target mutation. In one embodiment, the panel comprises 5-200 individual probe molecules, each complementary to a specific target mutation. In one embodiment, the panel comprises 5-100 individual probe molecules, each complementary to a specific target mutation. In one embodiment, the panel comprises 5-50 individual probe molecules, each complementary to a specific target mutation.

In one embodiment, there may be a plurality of probe molecules specific to the same mutation. In one embodiment, there may be only one probe molecule specific to each mutation of the panel.

In one embodiment, there is provided a panel, wherein the panel comprises a plurality of probe molecules wherein one or more probes are complementary to an EGFR mutation, one or more probes are complementary to a KRAS mutation, one or more probes are complementary to a ERBB2/HER2 mutation, one or more probes are complementary to a EML4-ALK mutation, one or more probes are complementary to a ROS1 mutation, one or more probes are complementary to a RET mutation and one or more probes are complementary to a MET mutation.

In one embodiment, there is provided a panel, wherein the panel comprises a plurality of probe molecules wherein one or more probes may be complementary to an EGFR mutation, one or more probes may be complementary to a KRAS mutation, one or more probes may be complementary to a ERBB2/HER2 mutation, one or more probes may be complementary to a EML4-ALK mutation, one or more probes may be complementary to a ROS1 mutation, one or more probes may be complementary to a RET mutation and one or more probes may be complementary to a MET mutation.

In one embodiment, there is provided a panel of probes selective for one or more EGFR, KRAS, BRAF, ERBB2/HER2, EML4-ALK, ROS1, RET, MET mutations.

In one embodiment, there is provided a panel of probe molecules selective for EGFR mutations.

In one embodiment, there is provided a panel of probe molecules selective for KRAS mutations.

In one embodiment, there is provided a panel of probe molecules selective for BRAF mutations.

In one embodiment, there is provided a panel of probe molecules selective for ERBB2/HER2 mutations.

In one embodiment, there is provided a panel of probe molecules selective for EML4-ALK mutations.

In one embodiment, there is provided a panel of probe molecules selective for ROS1 mutations.

In one embodiment, there is provided a panel of probe molecules selective for RET mutations.

In one embodiment, there is provided a panel of probe molecules selective for NTRK mutations.

In one embodiment, there is provided a panel of probe molecules selective for ROS1 mutations.

In one embodiment, there is provided a panel of probe molecules selective for MET exon 14 mutations.

In one embodiment, there is provided a panel comprising a plurality of probe molecules selective for one or more coding sequences (CDSs).

In one embodiment, there is provided a method of detecting one or more mutations using one or more of the previously described panels.

In one embodiment, there is provided a method of detecting the presence or absence or one or more mutations using one or more of the previously described panels.

In one embodiment, there is provided a kit comprising a panel, which may be as previously or subsequently described, in combination with one or more reagents, which may be as previously or subsequently described.

The person skilled in the art will appreciate that embodiments of kits that disclose A₀, include within their scope embodiments wherein there is a panel comprising a plurality of A₀.

The person skilled in the art will appreciate that the disclosure of the application further encompasses embodiments of panels including capture oligonucleotides B₀. This includes embodiments wherein A₀ and B₀ are regions of the same oligonucleotide C₀.

In one embodiment, there is provided a methylation detection panel.

In one embodiment, there is provided a methylation detection kit.

### Companion Diagnostics

The methods of the present invention can be used to detect specific genetic markers in a sample which may be used to help guide the selection of appropriate therapy. These markers may be tumour-specific mutations, or may be wild-type genomic sequences, and may be detected using tissue, blood or any other patient sample type. The markers may be epigenetic markers.

### Resistance Monitoring

Repeated testing of patient samples during treatment of disease may allow early detection of developed resistance to therapy. As an example of this application is in non-small cell lung carcinoma (NSCLC), in which epidermal growth factor receptor (EGFR) inhibitors (e.g. gefitinib, erlotinib) are commonly used as first line treatments. During treatment the tumour can often develop mutations in the EGFR gene (e.g. T790M, C797S) which confer resistance to the drug. Early detection of these mutations may allow for transfer of the patient onto alternative therapies (e.g. Tagrisso). Epigenetic changes to the DNA of a patient can indicate the development of resistance.

Typically patients being monitored for resistance onset can be too sick for repeated tissue biopsy to be carried out. Repeated tissue biopsy may also be expensive, invasive and carries associated risks. It is preferable to test from blood, but there may be very low copy numbers of the mutations of interest in a reasonable blood drawn sample. Monitoring therefore requires sensitive testing from blood samples using a method of the present invention in which the method is simple and cost effective to carry out such that it can be regularly performed.

### Recurrence Monitoring

In this application example, patients who have been declared free of disease following treatment may be monitored over time to detect the recurrence of disease. This needs to be done non-invasively and requires sensitive detection of target sequences from blood samples. By using the method of the present invention, it provides a simple and low-cost method that can be regularly performed. The sequences targeted may be generic mutations known to be common in the disease of interest, or can be custom panels of targets designed for a specific patient based on detection of variants in the tumour tissue prior to remission.

### Minimal Residual Disease (MRD) Monitoring

For some cancers there are residual cancer cells that remain in a patient after treatment, it is a major cause of relapse in cancer and leukaemia. MRD monitoring and testing has several important roles: determining whether treatment has eradicated the cancer or whether traces remain, comparing the efficacy of different treatments, monitoring patient remission status as well as detecting recurrence of leukaemia, and choosing the treatment that will best meet those needs.

### Screening

Population screening for early detection of disease has been a long-held goal, particularly in cancer diagnostics. The challenge is two-fold: the identification of panels of markers which allow confident detection of disease without too many false negatives, and the development of a method with sufficient sensitivity and low enough cost. The methods of the present invention could be used to address larger panels of mutations than PCR-based tests but with a much simpler workflow and lower cost than sequencing-based diagnostics.

### Organ Transplant Rejection

When a transplanted organ is rejected by the recipient, the DNA from this organ is shed into the recipient's bloodstream. Early detection of this DNA would allow early detection of rejection. This could be achieved using custom panels of donor-specific markers, or by using panels of variants known to be common in the population, some of which will be present in the donor and some in the recipient. Routine monitoring of organ recipients over time could be enabled by the low cost and simple workflow of the present invention disclosed herein.

### Non-Invasive Prenatal Testing (NIPT)

It has long been known that foetal DNA is present in maternal blood, and the NIPT market is now quite saturated with companies using sequencing the identify mutations and count copy numbers of specific chromosomes to enable detection of foetal abnormalities. The methods of the present invention as disclosed herein have the ability to detect mutations at very low allele fractions, potentially allowing earlier detection of foetal DNA. Identification of common mutations in a given population would allow assays to be developed that target mutations that may be present in either the maternal or foetal DNA or to allow detection of abnormalities at an earlier stage of pregnancy.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

The person skilled in the art will understand that references to ‘partially digested strand A₁’ may refer to the single-stranded oligonucleotide formed by progressive digestion of A₀ when hybridised to a target analyte sequence, in the 3′-5′ direction until the strands dissociate due to lack of complementarity.

The person skilled in the art will understand that references to ‘partially double-stranded’ nucleic acids may refer to nucleic acids wherein one or more portions are double-stranded and one or more portions are single-stranded.

The person skilled in the art will understand that references to ‘substantially double-stranded’ nucleic acids may refer to nucleic acids wherein one or more portions are double-stranded and one or more smaller portions are single-stranded.

### Example 8: Methylation Detection Utilising Target Enrichment

### 1. Bead Preparation—Blocking Step

A volume of 1 uL of beads (ThermoFisher Dynabeads MyOne Streptavidin T1 cat. 65601) per sample were put into a 1.5 mL Eppendorf tube. Up to 100 μL of beads can be blocked with 1 mL of blocking solution.

This tube was then placed on top of a magnet.

The storage buffer was removed by aspiration.

To the tube containing the beads 1 mL blocking buffer was added (1×Phosphate Buffered Saline (PBS) with 1 ug/mL tRNA)

This tube was then rotated at 40 rpm at room temperature (RT) for 30 min

### 2. Oligonucleotide Annealing

Dilutions of oligonucleotides in 1×AB buffer were prepared (Tris HCl pH=7.5 1 mM, NaCl 50 mL, EDTA 0.2 mM)
Capture oligonucleotide B₀ 100 nM
Probe A₀ 40 nM
Unmethylated strand 20 fM-2 nM
+/−Methylated strand—1 fM-100 pM
A 50 uL mixture of the oligonucleotides was prepared per 1 μL of beads

(SEQ ID NO 24)
Capture oligonucleotide:
5'-
/5biotin/TITTTAGGCGGGAGACATATGGGGAGCCCAGTGG-3'
(SEQ ID NO 25)
Probe:
5'-
/5Phos/A*TGTTCGATGAGCTTTGACAATACTTGATCGATGCAGATA
TAGGATGTTGCGACCATCACGTATGCCATCACGTATGCTTCCTGGGGA
CATTT/3SpC3/-3'
where * represents a phosphorothioate bond,
/3SpC3/ represents C3 Spacer
(SEQ ID 26)
Unmethylated oligonucleotide:
5'-CTCAGCGTACCCTTGTCCCCAGGAAGCATACGTGATGGCATACGT
GATGGCTGGTGTGGGCTCCCCATATGTCTCCCGCCTTCTGGGCAT-3'
(SEQ ID 27)
Methylated oligonucleotide:
5'- CTCAGCGTACCCTTGTCCC/iMe-
dC/AGGAAGCATACGTGATGGCATACGTGATGGCTGGTGTGGGCTCCC
CATATGTCTCCCGCCTTCTGGGCAT-3'

Where/iMe-dC/is 5-Methyl deoxycytidine

The resulting mixture was then incubated at 95° C. for 5 min followed by cooling to room temperature.

3. Attachment of oligonucleotides to the beads
a. Beads were spun down and placed on top of a magnet
b. The blocking solution was removed
c. 10 μL of 2×Binding buffer (TrisHCl pH=7.5 40 mM, NaCl 1M, EDTA 2 mM, Tween20 0.02%) was added per every 1 μL of beads
d. The beads were mixed with following ratio:
40 μL of 2×Binding buffer
10 μL of beads
50 μL of annealed oligonucleotides
The beads were then rotated at 40 rpm for 30 min at RT.
   4. Beads were then washed with 100 uL 1×Washing buffer (TrisHCl pH=7.5 20 mM, NaCl 0.5 M, EDTA 1 mM, Tween20 0.1%) three times.
   5. The wash buffer was then replaced with 100 uL 1×BFF5 buffer with 0.01% Triton-X
1×BFF5 with 0.01% Triton-X composition:
TrisHCl pH=7.0 10 mM
KCl 30 mM
MgCl₂ 5 mM
Triton-X 0.01%
   6. The 1×BFF5 0.01% Triton-X buffer was then removed followed by the addition of a pyrophosphorolysis (PPL) mixture to the beads (Kept at 4° C.). PPL mixture composition:
1×BFF1
10 U/mL Klenow (exo-)
100 U/mL E. coli Ligase
0.25 mM PPi
30 nM splint oligonucleotide
166.6 U/mL LpnPI
0.33×Enzyme Activator
Total volume 10 uL
This mixture was then incubated at 37° C. for 45 min.

(SEQ ID NO 28)
Splint oligonucleotide:
5'-TGTCAAAGCTCAGCTATCTGACGTGATTCGCAACAA-3'

### 7. Detection—RCA

A mixture corresponding to the following was prepared:
2.66× Isothermal buffer (53.2 mM Tris-HCl, 26.6 mM (NH₄)₂SO₄, 133 mM KCl, 5.32 mM MgSO₄,
0.266% Tween20, pH 8.8)
0.28 uM Primer mix 1
284.4 U/mL BST 2.0 WarmStart
14.67 U/mLTIPP
1.06 mM dNTPs
Syto82 dye 3 uM
1.25 uL of reaction mixture from point 2.
Total volume 11.25 uL

Primer mix 1:

(SEQ ID NO 29)
Fwd:
5'- T*C*GCAACATCCTATATCTGC -3'
(SEQ ID NO 30)
Rev:
5'- T*G*AGCTTTGACAATACTTGA -3'
where * represents a phosphorothioate bond

The mixture was then incubated at 62° C. for 90 min. Fluorescent measurements were taken every 1 minute. The results can be seen in FIG. 1A.

### Oligos in Solution

1. Oligonucleotide annealing
Dilutions of oligonucleotides in 1×AB buffer were prepared (Tris HCl pH=7.5 1 mM, NaCl 50 mL, EDTA 0.2 mM)
Capture oligonucleotide B₀ 100 nM
Probe A₀ 400 nM
Unmethylated strand 200 fM-20 nM
+/−Methylated strand—10 fM-1000 pM

A 50 uL mixture of the oligonucleotides was prepared per 1 μL of beads

(SEQ ID NO 24)
Capture oligonucleotide:
5'-/5biotin/T
TTTTTAGGCGGGAGACATATGGGGAGCCCAGTGG-3'
(SEQ ID NO 25)
Probe:
5'-
/5Phos/A*TGTTCGATGAGCTTTGACAATACTTGATCGATGCAGA
TATAGGATGTTGCGACCATCACGTATGCCATCACGTATGCTTCCTG
GGGACATTT/3SpC3/-3'
where * represents a phosphorothioate bond, /3SpC3/
represents C3 Spacer
(SEQ ID 26)
Unmethylated oligonucleotide:
5'-CTCAGCGTACCCTTGTCCCCAGGAAGCATACGTGATGGCATACGT
GATGGCTGGTGTGGGCTCCCCATATGTCTCCCGCCTTCTGGGCAT-3'
(SEQ ID 27)
Methylated oligonucleotide:
5'- CTCAGCGTACCCTTGTCCC/iMe-
dC/AGGAAGCATACGTGATGGCATACGTGATGGCTGGTGTGGGCTCCC
CATATGTCTCCCGCCTTCTGGGCAT-3'
Where /iMe-dC/ is 5-Methyl deoxycytidine

The resulting mixture was then incubated at 95° C. for 5 min followed by cooling to room temperature.

2. PPL mixture composition:
1×BFF1
10 U/mL Klenow (exo-)
100 U/mL E. coli Ligase
0.25 mM PPi
30 nM splint oligonucleotide
166.6 U/mL LpnPI
0.33×Enzyme Activator
1 uL of oligos from point 1.
Total volume 10 uL

This mixture was then incubated at 37° C. for 45 min.

(SEQ ID NO 28)
Splint oligonucleotide:
5'-TGTCAAAGCTCAGCTATCTGACGTGATTCGCAACAA-3'

### 3. Detection—RCA

A mixture corresponding to the following was prepared:
2.66× Isothermal buffer (53.2 mM Tris-HCl, 26.6 mM (NH₄)₂SO₄, 133 mM KCl, 5.32 mM MgSO₄,
0.266% Tween20, pH 8.8)
0.28 uM Primer mix 1
284.4 U/mL BST 2.0 WarmStart
14.67 U/mLTIPP
1.06 mM dNTPs
Syto82 dye 3 uM
1.25 uL of reaction mixture from point 2.
Total volume 11.25 uL

Primer mix 1:

(SEQ ID NO 29)
Fwd:
5'- T*C*GCAACATCCTATATCTGC -3'
(SEQ ID NO 30)
Rev:
5'- T*G*AGCTTTGACAATACTTGA -3'
where * represents a phosphorothioate bond

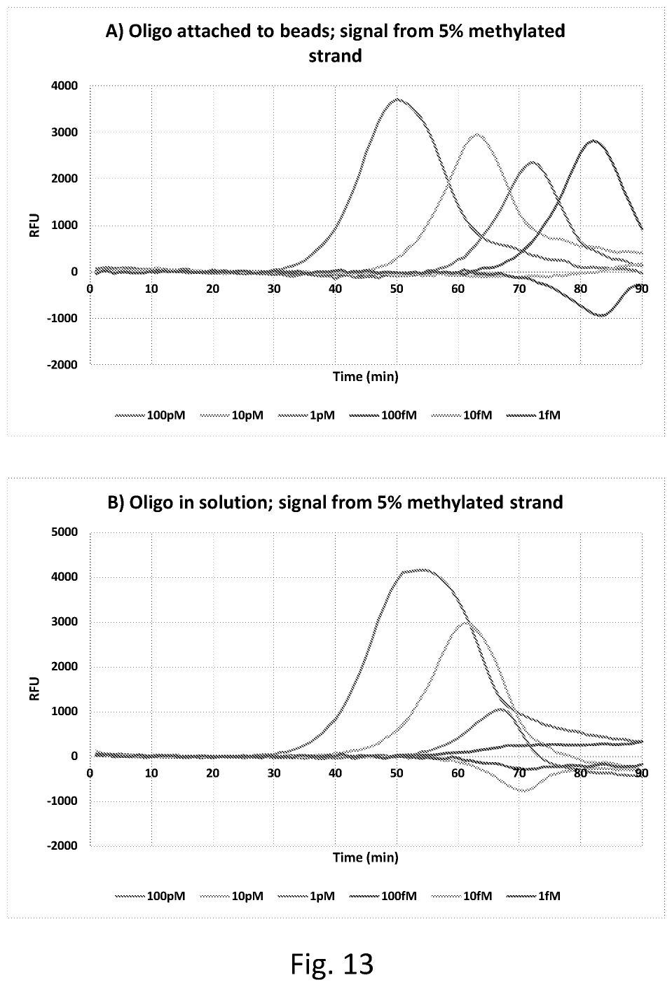

The mixture was then incubated at 62° C. for 90 min. Fluorescent measurements were taken every 1 minute. The results can be seen in FIG. 13B.

1. A method for the detection of a polynucleotide target sequence in a sample, comprising the steps of:
Introducing a sample comprising one or more nucleic acid analytes to a first reaction mixture comprising:
a single-stranded probe oligonucleotide A₀ including a region complementary to a target nucleic acid sequence;
a capture oligonucleotide B₀ comprising a region complementary to a region adjacent to the target nucleic acid sequence and a capture moiety; and
a solid support,
allowing A₀ and B₀ to hybridise to the nucleic acid analyte;
introducing the hybridised probes A₀ to a second reaction mixture comprising a pyrophosphorolysing enzyme and a ligase, wherein A₀ is pyrophosphorolysed in the 3′-5′ direction from the 3′ end to create at least a partially digested strand A₁ and A₁ undergoes ligation to form A₂; and
detecting a signal derived from the products of the previous step, wherein the products are A₂ or a portion thereof, or multiple copies of A₂ or multiple copies of a portion thereof, and inferring therefrom the presence or absence of the polynucleotide target sequence in the analyte.

2. The method of claim 1 wherein the second reaction mixture further comprises an inorganic pyrophosphatase.

3. A method as claimed in claim 1 wherein the second reaction mixture further comprises a source of pyrophosphate ions.

4. A method as claimed in claim 1 wherein B₀ is attached to the solid support prior to step (b).

5. A method as claimed in claim 1 wherein B₀ is attached to the solid support between steps (b) and (c).

6. A method as claimed in claim 1 wherein following attachment of B₀ to the solid support and hybridisation of A₀ and B₀ to the target, the supernatant and thus any A₀ which are not hybridised to the target nucleic acid analyte are removed from the reaction mixture.

7. A method as claimed in claim 1 wherein the capture oligonucleotide is complementary to a region that is within 100 nucleotides of the target nucleic acid sequence.

8. A method as claimed in claim 7 wherein the capture oligonucleotide is complementary to a region that is within 50 nucleotides of the target nucleic acid sequence.

9. A method as claimed in claim 8 wherein the capture oligonucleotide is complementary to a region that is within 10 nucleotides of the target nucleic acid sequence.

10. A method as claimed in claim 5 wherein A₀ and B₀ are different regions of the same oligonucleotide C₀ and wherein prior to (c), C₀ is cleaved to leave a 3′ end which is complementary to the target nucleic acid sequence.

11. A method as claimed in claim 1 wherein B₀ is covalently attached to the solid support via the capture moiety of B₀.

12. A method as claimed in claim 1 wherein B₀ is non-covalently attached to the solid support via the capture moiety of B₀.

13. A method as claimed in claim 12 wherein the capture moiety of B₀ comprises an oligonucleotide sequence and the solid support comprises oligonucleotides bearing the complementary sequence.

14. A method as claimed in claim 11 wherein the capture moiety comprises a chemical modification to B₀ and B₀ is attached to the solid support via an interaction between the chemical modification and the solid support.

15. A method as claimed in claim 14 wherein the capture moiety is biotin and the solid support further comprises streptavidin.

16. A method as claimed in claim 1 wherein the first reaction mixture comprises multiple different oligonucleotides A₀ and B₀ and wherein the successfully hybridised A₀ oligonucleotides are simultaneously enriched.

17. A method as claimed in claim 5 wherein prior to, during, or after step (c), A₀, the target nucleic acid, and optionally B₀ are released from the solid support.

18. A method as claimed in claim 17 wherein the capture moiety is an oligonucleotide region and wherein release is performed through heating of the reaction mixture or through the cleavage of oligonucleotide B₀.

19. A method as claimed in claim 18 wherein B₀ is cleaved chemically or enzymatically.

20. A method as claimed in claim 19 wherein B₀ is cleaved by a restriction enzyme or a flap endonuclease.

21. A method as claimed in claim 19 wherein B₀ is wherein B₀ comprises a photocleavable linker and A₀, B₀ and the target nucleic acid are released from the solid support by cleavage of this linker.

22. A method as claimed in claim 1 wherein each capture oligonucleotide is resistant to pyrophosphorolysis.

23. As method as claimed in claim 1 wherein the final step of the method comprises the introduction of the products of the previous step with a reaction mixture comprising at least one single-stranded primer oligonucleotide, deoxynucleotide triphosphates (dNTPs) and optionally an amplification enzyme.

24. A method as claimed in claim 23 wherein the pyrophosphorolysis enzyme of the second reaction mixture is the same enzyme which performs amplification and thus the further reaction mixture does not comprise an amplification enzyme.

25. A method as claimed in claim 1 wherein the method comprises the introduction providing a reaction mixture comprising:
a ligation probe oligonucleotide C which has a 5′ phosphate, a splint oligonucleotide D which is complementary to the 3′ end of A₁ and the 5′ end of C, and the partially digested strand A₁ is ligated at the 3′ end to the 5′ end of C to form oligonucleotide A₂; and
hairpin oligonucleotide 1 (HO1) and hairpin oligonucleotide 2 (HO2), each of which comprises a fluorophore and quencher such that when each oligonucleotide remains in a hairpin configuration the fluorophore and quencher are in contact with each other;
wherein HO1 is designed such that A₂ anneals to it, opening the ‘hairpin’ structure and separating the fluorophore from the quencher, and the now ‘open’ HO1 anneals to HO2, opening the ‘hairpin’ structure and separating the fluorophore from the quencher.

26. A method as claimed in claim 1 wherein the method further comprises the introduction of providing a reaction mixture comprising:
a ligation probe oligonucleotide C which has a 5′ phosphate, a splint oligonucleotide D which is complementary to the 3′ end of A₁ and the 5′ end of C, and the partially digested strand A₁ is ligated at the 3′ end to the 5′ end of C to form oligonucleotide A₂.

27. A method as claimed in claim 26 wherein the reaction mixture is combined with a further reaction mixture comprising:
an oligonucleotide A comprising a substrate arm, a partial catalytic core and a sensor arm; and
an oligonucleotide B comprising a substrate arm, a partial catalytic core and a sensor arm; and a substrate comprising a fluorophore-quencher pair;
wherein the sensor arms of oligonucleotides A and B are complementary to flanking regions of A₂ such that in the presence of A₂ oligonucleotides A and B are combined to form a catalytically, multicomponent nucleic acid enzyme (MNAzyme).

28. A method as claimed in claim 27 wherein the further reaction mixture comprises a partially double-stranded nucleic acid construct wherein:
one strand comprises at least one RNA base, at least one fluorophore and wherein a region of this strand is complementary to a region of A₂ and wherein this strand may be referred to as the ‘substrate’ strand;
the other stand comprises at least one quencher and wherein a region of this strand is complementary to a region of A₂ adjacent to that which the substrate strand is complementary to, such that in the presence of A₂ the partially stranded nucleic acid construct becomes substantially more double-stranded;
wherein in the process of becoming substantially more double-stranded the substrate strand of the double-stranded nucleic acid construct is cut at the RNA base, resulting in fluorescence due to the at least one quencher of the ‘other’ strand no longer being in close enough proximity to that of the at least one fluorophore of the substrate strand.

29. A method as claimed in claim 27 wherein the method further comprises the introduction of a reaction mixture comprising:
one or more ligases; and
two or more LCR probe oligonucleotides that are complementary to adjacent sequences on A₂, wherein when the probes are successfully annealed to A₂ the 5′ phosphate of one LCR probe is directly adjacent to the 3′OH of the other LCR probe.

30. A method as claimed in claim 27 wherein the method further comprises the introduction of a reaction mixture comprising:
a splint oligonucleotide, comprising a fluorophore-quencher pair, which is complementary to A₂;
a double strand specific DNA digestion enzyme;
wherein, in the presence of A₂, the splint oligonucleotide is digested such that the fluorophore-quencher pair are separated and a fluorescent signal, and hence the presence of A₂, is detectable.

31. A method as claimed in claim 1, wherein either:
A₁ is circularised through ligation of its 3′ and 5′ ends to create A₂; or
the second reaction mixture further comprises a ligation probe oligonucleotide C and that the ligation A₁ undergoes to form A₂ is ligation of the 3′ end of A₁ to the 5′ end of C.

32. A method as claimed in claim 31, further characterised in that the oligonucleotide C further comprises a 3′ or internal modification protecting it from 3′-5′ exonuclease digestion.

33. A method as claimed in claim 31, further characterised in that one of the reaction mixtures further comprises a splint oligonucleotide D that is unable to undergo extension against A₁ by virtue of either a 3′ modification or through a mismatch between the 3′ end of D and the corresponding region of A₁.

34. A method as claimed in claim 1 characterised in that detection is achieved using one or more oligonucleotide fluorescent binding dyes or molecular probes, wherein an increase in signal over time resulting from the generation of amplicons of A₂ is used to infer the concentration of the target sequence in the analyte.

35. A method as claimed in claim 1 further characterised in that multiple probes A₀ and multiple capture oligonucleotides B₀ are employed, wherein each A₀ is selective for a different target sequence and includes an identification region, wherein each B₀ comprises a region complementary to a region adjacent to a target sequence and further characterised in that the amplicons of A₂ include the identification region and therefore the target sequences present in the analyte, are inferred through the detection of the identification region(s).
